(19) United States
(12) Patent Application Publication
(10) Pub. No.: US 2025/0268948 A1
(43) Pub. Date: Aug. 28, 2025

(54) COMPOSITIONS AND METHODS FOR TREATING POST ACUTE SEQUELAE OF SARS-COV-2 INFECTION (LONG COVID)

(71) Applicant: VITRO BIOPHARMA, INC., Golden, CO (US)

(72) Inventors: James R. MUSICK, Golden, CO (US); Caroline MOSESSIAN, Golden, CO (US); Tiana STATES, Golden, CO (US)

(21) Appl. No.: 18/856,490

(22) PCT Filed: Apr. 12, 2023
(86) PCT No.: PCT/US2023/018357

§ 371 Date: Oct. 11, 2024

Related U.S. Application Data
(60) Provisional application No. 63/362,864, filed on Apr. 12, 2022.

(51) Int. Cl. A61K 35/51; A61K 31/05; A61K 31/12; A61K 31/167; A61K 31/19; A61K 31/352; A61K 33/00; A61K 35/28; A61K 38/08; A61K 38/15; A61K 47/22; A61P 31/14

(52) U.S. Cl. CPC A61K 35/51; A61K 31/05; A61K 31/12; A61K 31/167; A61K 31/19; A61K 31/352; A61K 33/00; A61K 35/28; A61K 38/08; A61K 38/15; A61K 47/22; A61P 31/14

(57) ABSTRACT

Disclosed are methods and compositions for treating a subject with Long COVID (e.g., Post-Acute Sequelae of SARS CoV-2 (PASC)).

### BACKGROUND

Post-acute COVID-19 symptoms include breathing difficulties, fatigue, “brain fog”, autonomic nervous system disfunction, loss of smell and taste as well as multiple additional symptoms that result in debilitation, disability, and significant cost burdens to the patient and the global health care systems. There are presently no treatments known to be effective in the treatment of long COVID.

### SUMMARY OF THE INVENTION

The present disclosure provides methods of treating a subject with Post-Acute Sequelae of SARS CoV-2 (PASC) and compositions for use thereof.

A first aspect of the disclosure features a method of treating a subject with Post-Acute Sequelae of SARS CoV-2 (PASC) including administering to the subject an effective amount of a composition including: (a) a plurality of stem cells; (b) a plurality of isolated exosomes about 80-200 nanometers (nm) in diameter, wherein the isolated exosomes are derived from the stem cells; (c) stem cell secretome-conditioned cell culture medium; (d) exosome-depleted stem cell secretome-conditioned cell culture medium; and/or (e) resveratrol, curcumin, and/or quercetin.

In some aspects, the composition is administered intravenously, intra-articularly, intramuscularly, intranasally, intrathecally, or by oral administration. In some aspects, the composition is administered intravenously. In some aspects, the composition is administered intravenously by infusion.

In some aspects, the composition is administered in a volume of about 0.5 milliliters (mL) to about 15 mL (e.g., 0.5 mL, 0.6 mL, 0.7 mL, 0.8 mL, 0.9 mL, 1 mL, 3 mL, 5 mL, 7 mL, 10 mL, 11 mL, 12 mL, 13 mL, 14 mL, or 15 mL). In some aspects, the composition is administered in a volume of about 1 mL to about 10 mL (e.g., 1 mL, 2 mL, 3 mL, 4 mL, 5 mL, 6 mL, 7 mL, 8 mL, 9 mL, or 10 mL).

In some aspects, the composition is administered in a volume of about 1 mL, about 2 mL, about 3 mL, about 4 mL, about 5 mL, or about 6 mL.

In some aspects, the composition is administered in a bolus or is administered over a period of about 1 minute to about 1 hour (e.g., 1 minute, 10 minutes, 20 minutes, 30 minutes, 40 minutes, 50 minutes, or 1 hour). In some aspects, the composition is administered over a period of about 1 minute to about 30 minutes (e.g., 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, or 30 minutes). In some aspects, the composition is administered over a period of about 1 minute to about 10 minutes (e.g., 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, or 10 minutes).

In some aspects, the composition is administered at a frequency of once every one, two, three, four, five, six, seven, eight, nine, ten, eleven, or twelve months. In some aspects, the composition is administered at a frequency of once every six to twelve months. In some aspects, the composition is administered at a frequency of once every month. In some aspects, the composition is administered at a frequency of once every two months. In some aspects, the composition is administered at a frequency of once every three months. In some aspects, the composition is administered at a frequency of once every four months. In some aspects, the composition is administered at a frequency of once every five months. In some aspects, the composition is administered at a frequency of once every six months. In some aspects, the composition is administered at a frequency of once every seven months. In some aspects, the composition is administered at a frequency of once every eight months. In some aspects, the composition is administered at a frequency of once every nine months. In some aspects, the composition is administered at a frequency of once every ten months. In some aspects, the composition is administered at a frequency of once every eleven months. In some aspects, the composition is administered at a frequency of once every twelve months.

In some aspects, the composition includes resveratrol, curcumin, and/or quercetin and is administered at a frequency of once every one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty days. In some aspects, the composition including resveratrol, curcumin, and/or quercetin is administered at a frequency of once every day. In some aspects, the composition including resveratrol, curcumin, and/or quercetin is administered at a frequency of once every two days. In some aspects, the composition including resveratrol, curcumin, and/or quercetin is administered at a frequency of once every three days. In some aspects, the composition including resveratrol, curcumin, and/or quercetin is administered at a frequency of once every four days. In some aspects, the composition including resveratrol, curcumin, and/or quercetin is administered at a frequency of once every five days. In some aspects, the composition including resveratrol, curcumin, and/or quercetin is administered at a frequency of once every six days. In some aspects, the composition including resveratrol, curcumin, and/or quercetin is administered at a frequency of once every seven days. In some aspects, the composition including resveratrol, curcumin, and/or quercetin is administered at a frequency of once every eight days. In some aspects, the composition including resveratrol, curcumin, and/or quercetin is administered at a frequency of once every nine days. In some aspects, the composition including resveratrol, curcumin, and/or quercetin is administered at a frequency of once every ten days. In some aspects, the composition is administered at said frequency for up to one month, six months, nine months, one year, or until resolution of one or more symptoms of PASC.

In some aspects, the stem cells are modified to increase an expression level of sirtuin 1 (Sirt-1), C—X—C motif chemokine receptor 4 (CXCR4), heat shock protein 70 (HSP70), octamer-binding transcription factor 3/4 (Oct 3/4), and/or fibroblast growth factor 21 (FGF-21) relative to unmodified stem cells. In some aspects, the modified stem cells include a nucleic acid vector, plasmid, circular RNA, or mRNA molecule including a nucleotide sequence encoding a fibroblast growth factor 2 (FGF-2) protein or substance P neuropeptide.

In some aspects, the method further includes, prior to administering the composition, contacting the stem cells with a stem cell activating agent. In some aspects, the stem cell activating agent is selected from the group consisting of a histone deacetylase 1 (HDAC1) inhibitor, a glycogen synthase kinase-3β (GSK-3Bβ) inhibitor, a neurokinin-1 (NK-1) receptor agonist, a fibroblast growth factor (FGF) protein, a nutraceutical, and lithium (Li).

In some aspects: (a) the HDAC1 inhibitor is selected from the group consisting of romidepsin (FK-228), vorinostat, romidepsin, belinostat, panobinostat, valproic acid (VPA), entinostat, curcumin, quercetin, and RG2833; (b) the GSK-3Bβ inhibitor is selected from the groups consisting of indirubin-3′-oxime, laduviglusib (CHIR-99821), and KY19382; (c) the NK-1 receptor agonist is substance P; (d) the FGF protein is FGF-2; and/or (d) the nutraceutical is selected from the group consisting of resveratrol, curcumin, and quercetin.

In some aspects: (a) contacting of the stem cells is with curcumin at a concentration of about 0.1 μM to about 1 μM; (b) contacting of the stem cells is with curcumin at a concentration of about 0.1 μM to about 1 μM and quercetin at a concentration of about 0.1 μM to about 1 μM; (c) contacting of the stem cells is with VPA at a concentration of about 20 μM to about 100 μM; (d) contacting of the stem cells is with Li at a concentration of about 10 μM to about 200 μM; (e) contacting of the stem cells is with VPA at a concentration of about 10 μM to about 125 μM and Li at a concentration of about 200 μM; (f) contacting of the stem cells is with VPA at a concentration of about 20 μM to about 250 μM and curcumin at a concentration of about 100 nM to about 1 μM; (g) contacting of the stem cells is with substance P at a concentration of about 1 nM to about 20 nM; (h) contacting of the stem cells is with FGF-2 at a concentration of about 3 ng/mL to 10 ng/mL; or (i) contacting of the stem cells is with curcumin at a concentration of about 500 nM and resveratrol at a concentration of about 50 nM to about 500 nM.

In some aspects: (a) contacting of the stem cells is with curcumin at a concentration of about 500 nM; (b) contacting of the stem cells is with curcumin at a concentration of about 500 nM and with quercetin at a concentration of about 317 nM; (c) contacting of the stem cells is with VPA at a concentration of about 38 μM; (d) contacting of the stem cells is with Li at a concentration of about 79 μM; (e) contacting of the stem cells is with VPA at a concentration of about 37 μM and Li at a concentration of about 200 μM; (f) contacting of the stem cells is with VPA at a concentration of about 250 μM and curcumin at a concentration of about 500 nM; (g) contacting of the stem cells is with substance P at a concentration of about 2.5 nM; (h) contacting of the stem cells is with FGF-2 at a concentration of about 4.2 ng/mL; or (i) contacting of the stem cells is with curcumin at a concentration of about 500 nM and resveratrol at a concentration of about 253 nM.

In some aspects, the HDAC1 inhibitor (e.g., romidepsin (FK-228), vorinostat, romidepsin, belinostat, panobinostat, VPA, entinostat, curcumin, quercetin, and RG2833) is at a concentration of about 1 nM to about 10 μM (e.g., 1 nM, 50 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or 10 PM). In some aspects, the HDAC1 inhibitor (e.g., romidepsin (FK-228), vorinostat, romidepsin, belinostat, panobinostat, VPA, entinostat, curcumin, quercetin, and RG2833) is at a concentration of about 100 nM to about 1 μM (e.g., 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, or 1 μM). In some aspects, the HDAC1 inhibitor (e.g., romidepsin (FK-228), vorinostat, romidepsin, belinostat, panobinostat, VPA, entinostat, curcumin, quercetin, and RG2833) is at a concentration of about 400 nM to about 600 nM (e.g., 400 nM, 425 nM, 450 nM, 475 nM, 500 nM, 525 nM, 550 nM, 575 nM, or 600 nM). In some aspects, the HDAC1 inhibitor (e.g., romidepsin (FK-228), vorinostat, romidepsin, belinostat, panobinostat, VPA, entinostat, curcumin, quercetin, and RG2833) is at a concentration of about 500 nM (e.g., 450 nM, 460 nM, 470 nM, 480 nM, 490 nM, 500 nM, 510 nM, 520 nM, 530 nM, 540 nM, or 550 nM).

In some aspects, the GSK-3Bβ inhibitor (e.g., indirubin-3′-oxime, laduviglusib (CHIR-99821), and KY19382) is at a concentration of about 1 nM to about 10 μM (e.g., 1 nM, 50 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or 10 μM). In some aspects, the GSK-3Bβ inhibitor (e.g., indirubin-3′-oxime, laduviglusib (CHIR-99821), and KY19382) is at a concentration of about 100 nM to about 1 μM (e.g., 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, or 1 μM). In some aspects, the GSK-3Bβ inhibitor (e.g., indirubin-3′-oxime, laduviglusib (CHIR-99821), and KY19382) is at a concentration of about 400 nM to about 600 nM (e.g., 400 nM, 425 nM, 450 nM, 475 nM, 500 nM, 525 nM, 550 nM, 575 nM, or 600 nM). In some aspects, the GSK-3Bβ inhibitor (e.g., indirubin-3′-oxime, laduviglusib (CHIR-99821), and KY19382) is at a concentration of about 500 nM (e.g., 450 nM, 460 nM, 470 nM, 480 nM, 490 nM, 500 nM, 510 nM, 520 nM, 530 nM, 540 nM, or 550 nM).

In some aspects, the nutraceutical (e.g., resveratrol, curcumin, and quercetin) is at a concentration of about 50 nM to about 500 nM (e.g., 50 nM, 100 nM, 150 nM, 200 nM, 250 nM, 300 nM, 350 nM, 400 nM, 450 nM, or 500 nM). In some aspects, the nutraceutical (e.g., resveratrol, curcumin, and quercetin) is at a concentration of about 500 nM (e.g., 450 nM, 460 nM, 470 nM, 480 nM, 490 nM, 500 nM, 510 nM, 520 nM, 530 nM, 540 nM, or 550 nM).

In some aspects, the contacting is for about 1 day to about 6 weeks (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days). In some aspects, the contacting is for about 1-3 weeks (e.g., 1 week, 10 days, 11 days, 2 weeks, 17 days, 18 days, 3 weeks). In some aspects, the contacting is for about 2 weeks (e.g., 13 days, 14 days, or 15 days). In some aspects, the contacting is for about 5 days. In some aspects, the contacting is for about 6 days. In some aspects, the contacting is for about 7 days.

In some aspects, the stem cell activating agent increases Sirt-1, CXCR4, HSP70, Oct 3/4, and/or FGF-21 expression levels in the stem cells relative to stem cells that are not in contact with the stem cell activating agent. In some aspects, the expression level of Sirt-1 is increased in the stem cells by about 200-fold to about 300-fold, the expression level of CXCR4 is increased in the stem cells by about 10-fold to about 20-fold, the expression level of HSP70 is increased in the stem cells by about 2-fold to about 20-fold, the expression level of Oct 3/4 is increased in the stem cells by about 10-fold to about 20-fold, and/or the expression level of FGF-21 is increased in the stem cells by about 5-fold to about 25-fold relative to stem cells that are not in contact with the stem cell activating agent. In some aspects, the stem cell activating agent increases proliferation and/or migration of the stem cells relative to stem cells that are not in contact with the stem cell activating agent.

In some aspects, the composition includes: (a) about 5×10⁷ to about 1×10⁸ of the stem cells; (b) a concentration of about 5×10⁹ to about 5×10¹⁰ of the exosomes per mL, wherein optionally said exosomes express cluster of differentiation markers CD9, CD63, CD81, CD29, CD44 and/or CD144; (c) about 100 pg/mL to about 5000 pg/mL of granulocyte-macrophage colony-stimulating factor (GM-CSF), macrophage inflammatory protein (MIP)-3 alpha (MIP-3a), IL-6, and IL-8 and/or about 10 pg/mL to about 1000 pg/mL of fractalkine and MIP-1; (d) about 50 nM to about 500 nM of resveratrol, curcumin, or quercetin; and/or (e) a pharmaceutically acceptable carrier, excipient, or diluent, wherein, optionally, the composition does not contain DMSO.

In some aspects, the excipient includes piperine. In some aspects, the composition further includes: (a) a cryopreservation medium; (b) a basal medium; and/or (c) a saline solution. In some aspects: (a) the cryopreservation medium is PRIME-XV® MSC FreeziS DMSO-Free medium; and/or (b) the basal medium is MCDB-131.

In some aspects, the composition includes about 7×10⁷ to about 1×10⁸ (e.g., 7×10⁷ to 8×10⁷, 8×10⁷ to 9×10⁷, or 9×10⁷ to 1×10⁸, e.g., 7×10⁷, 8×10⁷, 9×10⁷, or 1×10⁸) of the stem cells. In some aspects, the composition includes about 1×10⁸ (e.g., 9×10⁷, 9.1×10⁷, 9.2×10⁷, 9.3×10⁷, 9.4×10⁷, 9.5×10⁷, 9.6×10⁷, 9.7×10⁷, 9.8×10⁷, 9.9×10⁷, 1×10⁸, or 1.1×10⁸) of the stem cells.

In some aspects, the composition includes about 5×10⁵ to 5×10⁶ (e.g., 5×10⁵ to 1×10⁶, 5×10⁵ to 1.5×10⁶, 1×10⁶ to 2.5×10⁶, 1.5×10⁶ to 3×10⁶, 2×10⁶ to 5×10⁶, or 4×10⁶ to 5×10⁶, e.g., 5×10⁵, 6×10⁵, 7×10⁵, 8×10⁵, 9×10⁵, 1×10⁶, 1.5×10⁶, 2×10⁶, 2.5×10⁶, 3×10⁶, 3.5×10⁶, 4×10⁶, 4.5×10⁶, or 5×10⁶) of the stem cells per kilogram of the subject body weight. In some aspects, the composition includes about 1×10⁶ to 2.5×10⁶ (e.g., 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, or 2.5×10⁶) of the stem cells per kilogram of the subject body weight. In some aspects, the composition includes about 1×10⁶ of the stem cells per kilogram of the subject body weight.

In some aspects, the stem cells are mesenchymal stem cells (MSCs) or neural stem cells (NSCs). In some aspects, the MSCs are umbilical cord-derived MSCs (UC-MSCs). In some aspects, the UC-MSCs are ALLORX STEM CELLS®. In some aspects, the NSCs are nasal epithelium-derived MSCs.

In some aspects, the pharmaceutical composition includes a concentration of about 1×10¹⁰ to about 5×10¹⁰ (e.g., 5×10⁹ to 1.5×10¹⁰, 1×10¹⁰ to 3×10¹⁰, 2×10¹⁰ to 4×10¹⁰, 3×10¹⁰ to 5×10¹⁰, 1×10¹⁰ to 5×10¹⁰, or 2.5×10¹⁰ to 5×10¹⁰, e.g., 5×10⁹, 6×10⁹, 7×10⁹, 8×10⁹, 9×10⁹, 1×10¹⁰, 1.5×10¹⁰, 2×10¹⁰, 2.5×10¹⁰, 3×10¹⁰, 3.5×10¹⁰, 4×10¹⁰, 4.5×10¹⁰, or 5×10¹⁰) of the isolated exosomes per mL.

In some aspects: (a) the exosomes express CD9, CD63, and CD81; (b) the exosomes express CD44, CD29, and CD142; and/or (c) the exosomes do not express CD45, CD11b, CD14, CD19, CD34, CD79a, CD126, and human leukocyte antigen-DR isotype (HLD-DR).

In some aspects, the composition includes about 100 pg/mL to about 1000 pg/mL (e.g., 100 pg/mL to 500 pg/mL, 250 pg/mL to 750 pg/mL, or 500 pg/mL to 1000 pg/mL, e.g., 100 pg/mL, 150 pg/mL, 200 pg/mL, 250 pg/mL, 300 pg/mL, 350 pg/mL, 400 pg/mL, 450 pg/mL, 500 pg/mL, 600 pg/mL, 700 pg/mL, 800 pg/mL, 900 pg/mL, or 1000 pg/mL) of GM-CSF, MIP-3a, IL-6, and IL-8 and/or about 10 pg/mL to about 100 pg/mL (e.g., 10 pg/mL to 50 pg/mL, 25 pg/mL to 75 pg/mL, or 50 pg/mL to 100 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, or 100 pg/mL) of fractalkine and MIP-1. In some aspects, the composition includes about 1000 pg/mL of GM-CSF, MIP-3a, IL-6, and IL-8 and/or about 100 pg/mL of fractalkine and MIP-1.

In some aspects, prior to administration of the composition, the method includes administering a stem cell activating agent to the subject. In some aspects, the stem cell activating agent is selected from the group consisting of a HDAC1 inhibitor, a GSK-3Bβ inhibitor, an NK-1 receptor agonist, an FGF protein, a nutraceutical, Li, and a neural cell adhesion molecule (NCAM) modulator.

In some aspects: (a) the HDAC1 inhibitor is selected from the group consisting of romidepsin (FK-228), vorinostat, romidepsin, belinostat, panobinostat, VPA, entinostat, curcumin, quercetin, and RG2833; (b) the GSK-3Bβ inhibitor is selected from the groups consisting of indirubin-3′-oxime, laduviglusib (CHIR-99821), and KY19382; (c) the NK-1 receptor agonist is substance P; (d) the FGF protein is FGF-2; and/or (d) the nutraceutical is resveratrol, curcumin, or quercetin; and/or (e) the NCAM modulator is: (i) N-butylmannosamine; or (ii) an inhibitory nucleic acid molecule that targets ST8 alpha-N-acetyl-neuraminide alpha-2,8-sialyltransferase 1 (ST8Sial) and/or ST8 alpha-N-acetyl-neuraminide alpha-2,8-sialyltransferase 5 (ST8SiaV).

In some aspects, the HDAC1 inhibitor is administered to the subject in an amount sufficient to achieve a serum concentration of the HDAC1 inhibitor of about 0.1 nM to about 1000 nM (e.g., about 0.1 nM to 10 nM, about 1 nM to about 100 nM, about 10 nM to about 1000 nM, about 200 nM to about 500 nM, about 350 nM to about 700 nM, or about 500 nM to about 1000 nM, e.g., 01 nM, 0.5 nM, 1 nM, 5 nM, 10 nM, 20 nM, 30 nM, 40 nM, 50 nM, 60 nM, 70 nM, 80 nM, 90 nM, 100 nM, 125 nM, 150 nM, 175 nM, 200 nM, 225 nM, 250 nM, 275 nM, 300 nM, 325 nM, 350 nM, 375 nM, 400 nM, 425 nM, 450 nM, 475 nM, 500 nM, 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, or 1000 nM). In some aspects, the HDAC1 inhibitor is administered to the subject in an amount sufficient to achieve a serum concentration of the HDAC1 inhibitor of about 500 nM (e.g., 450 nM, 460 nM, 470 nM, 480 nM, 490 nM, 500 nM, 510 nM, 520 nM, 530 nM, 540 nM, or 550 nM).

In some aspects, the GSK-3Bβ inhibitor is administered to the subject in an amount sufficient to achieve a serum concentration of the GSK-3Bβ inhibitor of about 0.1 nM to about 1000 nM (e.g., about 0.1 nM to 10 nM, about 1 nM to about 100 nM, about 10 nM to about 1000 nM, about 200 nM to about 500 nM, about 350 nM to about 700 nM, or about 500 nM to about 1000 nM, e.g., 01 nM, 0.5 nM, 1 nM, 5 nM, 10 nM, 20 nM, 30 nM, 40 nM, 50 nM, 60 nM, 70 nM, 80 nM, 90 nM, 100 nM, 125 nM, 150 nM, 175 nM, 200 nM, 225 nM, 250 nM, 275 nM, 300 nM, 325 nM, 350 nM, 375 nM, 400 nM, 425 nM, 450 nM, 475 nM, 500 nM, 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, or 1000 nM). In some aspects, the GSK-3B3 inhibitor is administered to the subject in an amount sufficient to achieve a serum concentration of the GSK-3B3 inhibitor of about 500 nM (e.g., 450 nM, 460 nM, 470 nM, 480 nM, 490 nM, 500 nM, 510 nM, 520 nM, 530 nM, 540 nM, or 550 nM).

In some aspects, the NK-1 receptor agonist is administered to the subject in an amount sufficient to achieve a serum concentration of the NK-1 receptor agonist at a concentration of about 1 nM to about 5 nM (e.g., about 1 nM to about 4 nM, about 1 nM to about 3 nM, or about 2 nM to about 3 nM, e.g., 1 nM, 1.5 nM, 2 nM, 2.1 nM, 2.2 nM, 2.3 nM, 2.4 nM, 2.5 nM, 2.6 nM, 2.7 nM, 2.8 nM, 2.9 nM, 3 nM, 3.5 nM, 4 nM, 4.5 nM, or 5 nM). In some aspects, the NK-1 receptor agonist is administered to the subject in an amount sufficient to achieve a serum concentration of the NK-1 receptor agonist of about 2.5 nM.

In some aspects, the FGF protein is administered to the subject in an amount sufficient to achieve a serum concentration of the FGF protein of about 3 ng/mL to about 10 ng/mL (e.g., about 3 ng/mL to about 6 ng/mL or about 3.5 ng/mL to about 4.5 ng/mL, e.g., 3 ng/mL, 3.5 ng/mL, 3.6 ng/mL, 3.7 ng/mL, 3.8 ng/mL, 3.9 ng/mL, 4 ng/mL, 4.1 ng/mL, 4.2 ng/mL, 4.3 ng/mL, 4.4 ng/mL, 4.5 ng/mL, 5 ng/mL, 6 ng/mL, 7 ng/mL, 8 ng/mL, 9 ng/mL, or 10 ng/mL). In some aspects, the FGF protein is administered to the subject in an amount sufficient to achieve a serum concentration of the FGF protein of about 4.2 ng/mL.

In some aspects, the nutraceutical is administered to the subject in an amount sufficient to achieve a serum concentration of the nutraceutical of about 50 nM to about 500 nM (e.g., about 50 nM to about 200 nM, about 100 nM, to about 300 nM, about 200 nM to about 400 nM, about 300 nM to about 500 nM, e.g., 50 nM, 60 nM, 70 nM, 80 nM, 90 nM, 100 nM, 125 nM, 150 nM, 175 nM, 200 nM, 225 nM, 250 nM, 275 nM, 300 nM, 325 nM, 350 nM, 375 nM, 400 nM, 425 nM, 450 nM, 475 nM, or 500 nM). In some aspects, the nutraceutical is administered to the subject in an amount sufficient to achieve a serum concentration of the nutraceutical of about 500 nM (e.g., 450 nM, 460 nM, 470 nM, 480 nM, 490 nM, 500 nM, 510 nM, 520 nM, 530 nM, 540 nM, or 550 nM).

In some aspects, N-butylmannosamine is administered to the subject in an amount sufficient to achieve a serum concentration of N-butylmannosamine of about 1 mM to about 50 mM (e.g., about 1 mM to about 25 mM, about 5 mM to about 30 mM, about 10 mM, to about 35 mM, about 15 mM, to about 40 mM, about 20 mM to about 45 mM, or about 25 mM to about 50 mM, e.g., about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, or about 50 mM). In some aspects, N-butylmannosamine is administered to the subject in an amount sufficient to achieve a serum concentration of about 8 mM to 15 mM, about 8 mM, about 9 mM, about 10 mM, about 11 mM, about 12 mM, about 13 mM, about 14 mM, or about 15 mM.

In some aspects, the inhibitory nucleic acid molecule is a small interfering RNA (siRNA), an anti-sense oligonucleotide (ASO), a short hairpin RNA (shRNA), a micro RNA (miRNA), or a double stranded RNA (dsRNA).

In some aspects, the method includes administering the stem cell activating agent to the subject concurrently with or following administration of the composition. In some aspects, the stem cell activating agent is selected from the group consisting of an HDAC1 inhibitor, a GSK-3Bβ inhibitor, an NK-1 receptor agonist, an FGF protein, a nutraceutical, Li, and an NCAM modulator.

In some aspects: (a) the HDAC1 inhibitor is selected from the group consisting of romidepsin (FK-228), vorinostat, romidepsin, belinostat, panobinostat, VPA, entinostat, curcumin, quercetin, and RG2833; (b) the GSK-3Bβ inhibitor is selected from the groups consisting of indirubin-3′-oxime, laduviglusib (CHIR-99821), and KY19382; (c) the NK-1 receptor agonist is substance P; (d) the nutraceutical is resveratrol, curcumin, and/or quercetin; and/or (e) the NCAM modulator is: (i) N-butylmannosamine; or (ii) an inhibitory nucleic acid molecule that targets ST8Sial and/or ST8SiaV.

In some aspects, the HDAC1 inhibitor is administered to the subject in an amount sufficient to achieve a serum concentration of the HDAC1 inhibitor of about 0.1 nM to about 1000 nM (e.g., about 0.1 nM to 10 nM, about 1 nM to about 100 nM, about 10 nM to about 1000 nM, about 200 nM to about 500 nM, about 350 nM to about 700 nM, or about 500 nM to about 1000 nM, e.g., 01 nM, 0.5 nM, 1 nM, 5 nM, 10 nM, 20 nM, 30 nM, 40 nM, 50 nM, 60 nM, 70 nM, 80 nM, 90 nM, 100 nM, 125 nM, 150 nM, 175 nM, 200 nM, 225 nM, 250 nM, 275 nM, 300 nM, 325 nM, 350 nM, 375 nM, 400 nM, 425 nM, 450 nM, 475 nM, 500 nM, 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, or 1000 nM). In some aspects, the HDAC1 inhibitor is administered to the subject in an amount sufficient to achieve a serum concentration of the HDAC1 inhibitor of about 500 nM (e.g., 450 nM, 460 nM, 470 nM, 480 nM, 490 nM, 500 nM, 510 nM, 520 nM, 530 nM, 540 nM, or 550 nM).

In some aspects, the GSK-3Bβ inhibitor is administered to the subject in an amount sufficient to achieve a serum concentration of the GSK-3Bβ inhibitor of about 0.1 nM to about 1000 nM (e.g., about 0.1 nM to 10 nM, about 1 nM to about 100 nM, about 10 nM to about 1000 nM, about 200 nM to about 500 nM, about 350 nM to about 700 nM, or about 500 nM to about 1000 nM, e.g., 01 nM, 0.5 nM, 1 nM, 5 nM, 10 nM, 20 nM, 30 nM, 40 nM, 50 nM, 60 nM, 70 nM, 80 nM, 90 nM, 100 nM, 125 nM, 150 nM, 175 nM, 200 nM, 225 nM, 250 nM, 275 nM, 300 nM, 325 nM, 350 nM, 375 nM, 400 nM, 425 nM, 450 nM, 475 nM, 500 nM, 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, or 1000 nM). In some aspects, the GSK-3Bβ inhibitor is administered to the subject in an amount sufficient to achieve a serum concentration of the GSK-3Bβ inhibitor of about 500 nM (e.g., 450 nM, 460 nM, 470 nM, 480 nM, 490 nM, 500 nM, 510 nM, 520 nM, 530 nM, 540 nM, or 550 nM).

In some aspects, the NK-1 receptor agonist is administered to the subject in an amount sufficient to achieve a serum concentration of the NK-1 receptor agonist of about 1 nM to about 5 nM (e.g., about 1 nM to about 4 nM, about 1 nM to about 3 nM, or about 2 nM to about 3 nM, e.g., 1 nM, 1.5 nM, 2 nM, 2.1 nM, 2.2 nM, 2.3 nM, 2.4 nM, 2.5 nM, 2.6 nM, 2.7 nM, 2.8 nM, 2.9 nM, 3 nM, 3.5 nM, 4 nM, 4.5 nM, or 5 nM). In some aspects, the NK-1 receptor agonist is administered to the subject in an amount sufficient to achieve a serum concentration of the NK-1 receptor agonist of about 2.5 nM.

In some aspects, the FGF protein is administered to the subject in an amount sufficient to achieve a serum concentration of the FGF protein of about 3 ng/mL to about 10 ng/mL (e.g., about 3 ng/mL to about 6 ng/mL or about 3.5 ng/mL to about 4.5 ng/mL, e.g., 3 ng/mL, 3.5 ng/mL, 3.6 ng/mL, 3.7 ng/mL, 3.8 ng/mL, 3.9 ng/mL, 4 ng/mL, 4.1 ng/mL, 4.2 ng/mL, 4.3 ng/mL, 4.4 ng/mL, 4.5 ng/mL, 5 ng/mL, 6 ng/mL, 7 ng/mL, 8 ng/mL, 9 ng/mL, or 10 ng/mL). In some aspects, the FGF protein is administered to the subject in an amount sufficient to achieve a serum concentration of the FGF protein of about 4.2 ng/mL.

In some aspects, the nutraceutical is administered to the subject in an amount sufficient to achieve a serum concentration of the nutraceutical of about 50 nM to about 500 nM (e.g., about 50 nM to about 100 nM, about 75 nM to about 150 nM, about 100 nM to about 200 nM, about 150 nM to about 300 nM, about 200 nM, to about 400 nM, about 240 nM to about 260 nM, about 300 nM to about 500 nM, or about 400 nM to about 500 nM, e.g., 50 nM, 100 nM, 150 nM, 200 nM, 250 nM, 253 nM, 275 nM, 300 nM, 350 nM, 400 nM, 450 nM, or 500 nM). In some aspects, the nutraceutical is administered to the subject in an amount sufficient to achieve a serum concentration of the nutraceutical of about 500 nM (e.g., 450 nM, 460 nM, 470 nM, 480 nM, 490 nM, 500 nM, 510 nM, 520 nM, 530 nM, 540 nM, or 550 nM).

In some aspects, N-butylmannosamine is administered to the subject in an amount sufficient to achieve a serum concentration of N-butylmannosamine of about 1 mM to about 50 mM (e.g., about 1 mM to about 25 mM, about 5 mM to about 30 mM, about 10 mM, to about 35 mM, about 15 mM, to about 40 mM, about 20 mM to about 45 mM, or about 25 mM to about 50 mM, e.g., about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, or about 50 mM).

In some aspects, the inhibitory nucleic acid molecule is an siRNA, an ASO, an shRNA, a miRNA, or a dsRNA.

In some aspects, the subject is administered: (a) vorinostat in an amount of about 400 mg and/or in an amount sufficient to achieve geometric mean values of a maximum plasma concentration (Cₘₐₓ) and an area under the plasma concentration versus time curve (AUC_{0-inf}) of about 1.2±0.53 μM and about 6.0±2.0 μM*hr, respectively; (b) romidepsin in an amount of about 14 mg/m² IV over a 4-hour period, such as on days 1, 8, and 15 of a 28-day cycle and/or in an amount sufficient to achieve geometric mean values of a maximum plasma concentration (Cₘₐₓ) and an area under the plasma concentration versus time curve (AUC_{0-inf} of about 377 ng/mL and about 1549 ng*hr/mL, respectively; (c) belinostat in an amount of about 1000 mg/m² over a 30 minute period, such as on days 1-5 of a 21-day cycle; (d) panobinostat in an amount of about 20 mg every other day, such as on days 1, 3, 5, 8, 10, and 12 of a 21-day cycle; (e) valproic acid in an amount of about 10 to 60 mg/kg/day; (f) entinostat in an amount of about 2 mg/m² to about 12 mg/m² per day; (g) curcumin in an amount of about 1 g to about 8 g per day; (h) quercetin in an amount of about 250 mg to about 1000 mg per day; and/or (i) RG2833 in an amount of about 30 mg to about 240 mg per day.

In some aspects, the subject exhibits one or more symptoms of PASC, wherein optionally the one or more symptoms are present in the subject for at least four (4, 5, 6, 7, 8, 910, or more) weeks prior to administration of the composition. In some aspects, the one or more symptoms of PASC results from an administration of a SARS-CoV-2 vaccine. In some aspects, the one or more symptoms are selected from the group consisting of: cognitive impairment, joint pain, muscle pain, chest pain, stomach pain, headache, tachycardia, numbness, diarrhea, brain fog, fatigue, sleep impairment, dizziness when standing, shortness of breath, fever, skin rash, mood changes, loss of smell, exercise intolerance, and loss of taste.

In some aspects, the subject is suffering from an organ failure. In some aspects, the organ failure is a renal, hepatic, respiratory, or nervous system failure. In some aspects, the subject is being provided ventilatory support.

In some aspects, the subject exhibits an abnormal clinical laboratory measurement selected from the group consisting of: a lymphocyte count outside the range of 4-12×10⁹/L; a hemoglobin level less than 12.3 g/dL; a platelet level outside the range of 150-440×10⁹/L; a C3 level outside the range of 80-178 mg/dL; a C4 level greater than or equal to 12-42 mg/dL; a CRP level greater than 1 mg/dL; a d-dimer level greater than 500 ng/mL; an IgM level greater than 240 mg/dL; an IgG level greater than 1600 mg/dL; an IgA level greater than 450 mg/dL; a B cell count greater than or equal to 100-600×10⁶/L; a T cell count greater than or equal to 0.64-1.18×10⁹/L; a CD4/CD8 T cell ratio greater than 1.0; a natural killer (NK) cell count greater than 100×10⁶/L; an anti-nuclear antibody (ANA) titer greater than 1:160; and any positive autoantibody quantitation to autoantigens listed in Table 4.

In some aspects, the subject exhibits an abnormal clinical laboratory measurement selected from the group consisting of: a blood urea nitrogen (BUN) level greater than 25 mEq/L; a creatine level greater than 1.5 mEq/L; an aspartate transaminase (AST) level greater than 33 U/L; an alanine aminotransferase (ALT) level greater than 36 U/L; an alkaline phosphatase level greater than 115 IU/L; a white blood cell (WBC) count greater than 12×10^{9/}L; a lymphocyte count of about 800 cells per μL; a lactic acid level greater than 4.0 mmol/L; a procalcitonin level greater than 0.25 ng/mL; a lactate dehydrogenase (LDH) level greater than 240 U/L; a ferritin level greater than 284 ng/mL; a c-reactive protein (CRP) level greater than 1.0 mg/dL; and/or a d-dimer level greater than 500 ng/mL.

In some aspects, the subject exhibits an abnormal measurement during an arterial blood gas (ABG) test, wherein optionally the abnormal result includes an arterial blood pH level outside the range of 7.37-7.44, such as about 7.1; a partial pressure of carbon dioxide (pCO₂) outside the range of 34-43 mm Hg, such as about 60 mm Hg; a partial pressure of oxygen (pO₂) and fraction of inspired oxygen (FiO₂) ratio (pO₂/FiO₂) of less than 300, such as about 255; and a positive end-expiratory pressure (PEEP) of less than 30 cm H₂O.

In some aspects, administration of the composition returns the abnormal clinical laboratory measurement in the subject back to a baseline clinical laboratory measurement.

In some aspects, the baseline clinical laboratory measurement is selected from the group consisting: a BUN level of about 8-25 mEq/L; a creatine level of about 0.5-1.5 mEq/L; an AST level of about 8-33 U/L; an ALT level of about 4-36 U/L; an alkaline phosphatase level of about 25-115 U/L; a WBC count of about 4-12×10⁹/L with about 40-60% neutrophilic predominance and lymphocyte component of about 20-40%; a lactic acid level of about 2-4 mM; a procalcitonin level of about 0.1-0.25 ng/mL; an LDH level of about 50-240 U/L; a ferritin level of about 30-284 ng/mL; a CRP level of about 0.3-1.0 mg/dL; and/or a d-dimer level of less than 500 ng/mL.

In some aspects, the baseline clinical laboratory measurement is selected from the group consisting: a lymphocyte count of about 4-12×10⁹/L; a hemoglobin level of about 12.3-15.7 g/dL; a platelet level of about 150-440×10⁹/L; a C3 level of about 80-178 mg/dL; a C4 level of about 12-42 mg/dL; a CRP level of about 0.3-1 mg/dL; a d-dimer level of less than 500 ng/mL; an IgM level of about 40-240 mg/dL; an IgG level of about 600-1600 mg/dL; an IgA level of about 80-450 mg/dL; a B cell count of about 100-600×10⁶/L; a T cell count of about 0.64-1.18×10⁹/L; a CD4/CD8 T cell ratio of less than 1.0; a NK cell count greater than 100×10⁶/L; an ANA titer less than 1:160; and no detectable level of autoantigens listed in Table 4.

In some aspects, following administration of the composition, the subject exhibits a baseline ABG measurement. In some aspects, the baseline ABG measurement includes a pH level within the range of 7.35-7.45, such as about 7.435; a pCO₂ within the range of 37-45 mm Hg, such as about 43.3 mm Hg; a pO₂/FiO₂ greater than 300, such as about 316; and a PEEP of less than 30 cm H₂O.

In some aspects, administering to the subject the effective amount of the composition resolves one or more symptoms in the subject selected from the group consisting of: cognitive impairment, joint pain, muscle pain, chest pain, stomach pain, headache, tachycardia, numbness, diarrhea, brain fog, fatigue, sleep impairment, dizziness when standing, shortness of breath, fever, skin rash, mood changes, loss of smell, exercise intolerance, and loss of taste.

### BRIEF DESCRIPTION OF THE DRAWINGS

A complete understanding of the present disclosure may be obtained by reference to the accompanying drawings, when considered in conjunction with the subsequent detailed description, in which:

FIG. 1 is a microphotograph depicting cell migration using fluorescent readout and cell tracker green as a fluorescent marker of human MSCs.

FIG. 2 is an image illustrating MSC migration as percent closure as a function of time during live-cell data acquisition at different dosages of substance P.

FIG. 3 is a line graph representation illustrating migration of various human cell lines exposed to substance P.

FIG. 4 is a line graph representation illustrating less than 1 μM curcumin induced migration of human MSCs and 1 to 10 μM curcumin blocked migration due to apparent toxicity.

FIG. 5 is a line graph representation illustrating migration of MSCs induced by exposure to lithium alone (diamonds), VPA alone (squares) and VPA in the presence of 200 μM lithium (circles).

FIG. 6 is a line graph representation illustrating migration of MSCs, NSCs and colorectal CAFs induced by increasing VPA concentration in 200 μM lithium.

FIG. 7 is a line graph representation illustrating migration of human MSCs exposed to a combination of lithium and VPA with and without inhibition of MMP9 and CXCR4.

FIG. 8 is a line graph representation illustrating proliferation of different cell lines induced by increasing concentrations of FGF-b (FGF-2) after a 5 day exposure, FGF being the family of fibroblast growth factors.

FIG. 9 is a line graph representation illustrating proliferation of MSCs induced by lithium, VPA and VPA in 200 μM lithium after a 5 day exposure.

FIG. 10 is a line graph representation illustrating proliferation of MSCs and NSCs as a function of increasing VPA in 200 μM lithium after a 5 day exposure.

FIG. 11 is a bar graph representation illustrating qPCR used to measure target genes known to be subject to epigenetic regulation by HDAC inhibitors including VPA, Oct 3 and/or Oct4 (Oct 3/4), a pluripotency gene, Sirt-1, age-related gene, and FGF-21, whose expression is related to VPA-Li synergy.

FIG. 12 is a bar graph representation illustrating cytokine levels in cell culture media exposed to MSCs for 24 days and determined by microarray analysis.

FIG. 13 is a bar graph showing the expression levels of beta-actin, Oct 3/4, Sirtuin I, FGF-21, CXCR4, and Hsp70 in human MSCs exposed various epigenetic agents as determined by qPCR.

FIG. 14 is a line graph showing the activation of human MSC migration by various concentrations of quercetin in the presence of 500 nM curcumin and the inhibition of migration by 20 micromolar AMD3100 and 15 micromolar GM6001 which are specific inhibitors of CXCR4 and MMP9, respectively.

FIG. 15 is a line graph showing the activation of human MSC migration by various concentrations of resveratrol in the presence of 500 nM curcumin and the inhibition of migration by 20 micromolar AMD3100 and 15 micromolar GM6001 which are specific inhibitors of CXCR4 and MMP9, respectively.

FIG. 16 presents graphs showing the pulmonary parameters of a COVID-19 patient in ICU over seventy five days. The patient was treated with 100 million ALLORX STEM CELLS® at days 8, 12, and 18 following admission to the ICU.

FIG. 17 presents graphs showing the pulmonary function of a COVID-19 patient in ICU over seventy days. The patient was treated with 100 million ALLORX STEM CELLS® at days 8, 12, and 18 following admission to the ICU.

FIG. 18 presents graphs showing the clotting parameters of a COVID-19 patient in ICU over a one hundred days. The patient was treated with 100 million ALLORX STEM CELLS® at days 8, 12, and 18 following admission to the ICU.

FIG. 19 presents graphs showing renal function of a COVID-19 patient in ICU over a one hundred days. The patient was treated with 100 million ALLORX STEM CELLS® at days 8, 12, and 18 following admission to the ICU.

FIG. 20 is a graph showing the hepatic function of a COVID-19 patient in ICU over one hundred days. The patient was treated with 100 million ALLORX STEM CELLS® at days 8, 12, and 18 following admission to the ICU.

FIG. 21 is a graph showing the nutrition analysis of a COVID-19 patient in ICU over one hundred days. The patient was treated with 100 million ALLORX STEM CELLS® at days 8, 12, and 18 following admission to the ICU.

FIG. 22 is a diagram showing diafiltration of active cell culture in a stirred tank bioreactor to exchange the stem cell culture growth medium with basal medium as described in Example 13.

FIG. 23 is a diagram showing the stem cell secretome-conditioned cell culture medium harvesting step. This step occurs after an additional period of 72 hours in culture within the bioreactor following diafiltration (e.g., see FIG. 22). Harvesting of stem cell secretome-conditioned cell culture medium is further described in Example 13.

FIG. 24 is a series of photographs showing tri-lineage differentiation of UC-MSCs. Cellular markers are used to demonstrate UC-MSC differentiation into bone, cartilage and fat cells.

FIG. 25 shows an example of a Certificate of Analysis for manufactured UC-MSCs (e.g., ALLORX STEM CELLS®), as described herein.

FIG. 26. is a set of photographs showing an exemplary method for preparing cryopreserved UC-MSCs (e.g., ALLORX STEM CELLS®) for injection. The UC-MSCs are shipped in cryogenic conditions and require storage in liquid nitrogen prior to use. To prepare the UC-MSCs for administration to a subject (e.g., a subject with PASC), first thaw the cells at 37° C. immediately after being taken from the dewar using a water bath previously equilibrated to 37° C. Second, continuously swirl the vial of cells while submerged in water bath until they are thawed. Generally, it takes about 5 minutes to completely thaw the contents of a 5 ml vial. Third, in a sterile environment, use a sterile syringe with an attached sterile needle (e.g., an 18 gauge needle) to pull the cells out of the cryogenic vial. Change the needle before slowly pushing the cells into an IV bag to maintain cellular viability. Infuse the cells at about 50 drops per minute using a 100 ml normal saline drip. Any additional preparation step(s), such as pretreatment of the UC-MSCs, may be employed as described in the detailed description.

FIG. 27 is a graph showing growth and expansion characteristic of AD-MSCs, BM-MSCs, P-MSCs, and UCMSCs following pass 2 in cell culture. Black bars are cell count, bars labeled with “DT” are doubling times (DT): T In (Cf−Ci)/Ci where T is the time from subculture to detachment (Hrs), Ci is the initial cell count and Cf is the final cell count. Bars labeled with “V” are PI-determined viability (V).

FIG. 28 presents graphs showing immunomodulatory potency of UC-MSCs, AD-MSCs, P-MSCs, and UC-MSCs by an γ-IFN induced IDO activity assay.

FIG. 29 presents graphs showing that UC-MSCs have a significantly higher cellular ATP-content than the other ADMSCs, P-MSCs, and BM-MSCs.

FIG. 30 is a graph showing a comparison of migration by AD-MSCs, P-MSCs, BM-MSCs, and UC-MSCs into cell-free regions. Migration was determined as described in the Materials and Methods and the measured % closure of the occluded region is plotted as a function of time after exposure to 50 μg/ml substance P.

FIG. 31 is a graph showing the proliferation of AD-MSCs, P-MSCs, BM-MSCs, and UC-MSCs in varying levels of FBS added to a serum-free base. RFU's at day 3 minus day 1 following Presto Blue exposure are shown as a function of [FBS].

### DEFINITIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “about,” as applied to one or more values of interest, refers to a value that falls within 10%, 9%, 8%, 7%, 6%, 5%, 4%, 3%, 2%, 1%, or less in either direction (greater than or less than (+/−)) of a stated reference value, unless otherwise stated or otherwise evident from the context (except where such number would exceed 100% of a possible value).

As used herein, the term “exosome-depleted stem cell secretome-conditioned cell culture medium” refers to secretome-conditioned cell culture medium that has been processed to remove exosomes (e.g., exosomes that are of a specific size, e.g., 80-200 nm). The secretome is derived from stem cells (e.g., MSCs, UC-MSCs, or NSCs). Several standard laboratory techniques exist to remove exosomes from cell culture medium, such as differential ultracentrifugation, size exclusion chromatography, ultrafiltration, polyethylene glycol-based precipitation, immunoaffinity capture, or by using microfluidic devices. Exosome-depleted stem cell secretome-conditioned cell culture medium may contain other stem cell-derived biological material, such as proteins, lipids, and extracellular vesicles smaller than 80 nm or larger than 200 nm.

As used herein, the term “increased expression” refers to an expression level of an mRNA or protein (e.g., FGF-2 or substance P mRNA or protein) that is at least 5% higher (e.g., 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500% or more) than a control (e.g., the expression level of the mRNA or protein (e.g., FGF-2 or substance P) in an untreated cell (e.g., an untreated stem cell) or in an untreated subject), as determined by an objective assay (e.g., PCR, RT-PCR, qPCR, RT-qPCR, microarray analysis, Northern blot, MASSARRAY® technique, SAGE, RNA-sequencing, flow cytometry (FC), fluorescence-activated cell sorting (FACS) Western blot, enzyme-linked immunosorbent assay (ELISA), mass spectrometry (MS), immunofluorescence (IF), immunoprecipitation (IP), radioimmunoassay, dot blotting, high performance liquid chromatography (HPLC), surface plasmon resonance, optical spectroscopy, and immunohistochemistry (IHC)).

As used herein, the term “isolated exosome” refers to an exosome (or a population of exosomes) that was isolated from stem cell (e.g., MSC, UC-MSC, or NSC) secretome-conditioned cell culture medium. The isolated exosomes are 80 nm to 200 nm (e.g., 80 nm, 90 nm, 100 nm, 110 nm, 120 nm, 130 nm, 140 nm, 150 nm, 160 nm, 170 nm, 180 nm, 190 nm, or 200 nm) in diameter and express cluster of differentiation markers CD9, CD63, CD81, CD44, CD29, and CD142. Further, isolated exosomes do not express CD45, CD11b, CD14, CD19, CD34−, CD79a, CD126, and human leukocyte antigen-DR isotype (HLD-DR).

As used herein, the terms “umbilical cord-derived human mesenchymal stem cell” and “UC-MSC” refer to a class of multifunctional stem cells isolated and cultured from umbilical cord. They are capable of self-renewal, tri-lineage differentiation potential, and low immunogenicity.

As used herein, the terms “stem cell secretome-conditioned cell culture medium” and “secretome-conditioned cell culture medium” refer to cell culture medium that was previously incubated with a plurality of stem cells (e.g., MSCs, UC-MSCs, or NSCs, such as ALLORX STEM CELLS®). As a result, the cell culture medium comprises biological material (e.g., proteins, exosomes, and lipids) that may have been secreted from the stem cells.

As used herein, the term “stem cell activator” refers to any compound (e.g., chemical, small molecule, peptide, protein, or protein complex) that can stimulate or activate the stem cell proliferation and/or stem cell activation. Stem cell activators include, but are not limited to, an HDAC1 inhibitor (e.g., romidepsin (FK-228), vorinostat, romidepsin, belinostat, panobinostat, valproic acid (VPA), entinostat, curcumin, quercetin, and RG2833), a GSK-3B3 inhibitor (e.g., indirubin-3′-oxime, laduviglusib (CHIR-99821), and KY19382), a NK-1 receptor agonist (e.g., substance P), a FGF protein (e.g., FGF-2), a nutraceutical (e.g., resveratrol, curcumin, and quercetin), Li (e.g., lithium chloride), and an NCAM modulator. Exemplary NCAM modulators include N-butylmannosamine or inhibitory nucleic acid molecules (e.g., small interfering RNA (siRNA), an anti-sense oligonucleotide (ASO), a short hairpin RNA (shRNA), a micro RNA (miRNA), or a double stranded RNA (dsRNA)) that target ST8 alpha-N-acetyl-neuraminide alpha-2,8-sialyltransferase 1 (ST8Sial) and/or ST8 alpha-N-acetyl-neuraminide alpha-2,8-sialyltransferase 5 (ST8SiaV) in the subject, thereby reducing ST8Sial and ST8SiaV mRNA and protein expression. Standard laboratory techniques can be used to assess activation of stem cell proliferation (e.g., cell counts and 5-Bromo-2′-Deoxyuridine (BrdU) uptake assays) and migration (e.g., scratch and transwell migration assays).

### DETAILED DESCRIPTION

Although the following detailed description contains specific details for the purposes of illustration, those of ordinary skill in the art will appreciate that variations and alterations to the following details are within the scope of the disclosure. Accordingly, the exemplary embodiments of the disclosure described below are set forth without any loss of generality to, and without imposing limitations upon, the claimed invention.

Disclosed herein are materials, compositions and procedures for treating a subject experiencing persistent symptoms and/or delayed or long-term complications of SARS-CoV-2 (COVID-19) called Post-Acute Sequelae to SARS-CoV-2 infection (PASC) or to the SARS-CoV-2 vaccine beyond four weeks from the onset of symptoms or vaccine administration. Further embodiments include specific testing to determine the effectiveness of treatment, as assessed by the incidence of adverse events (AEs) and serious adverse events (SAEs) and assessment of improvement in fatigue and quality of life, change in baseline of lymphocytes, hemoglobin, platelets, complement component 3 (C3), complement component 4 (C4), C-reactive protein (CRP), D-dimer, IgM, IgG, IgA, B cells, T cells, CD4/CD8 T cell ratio, and natural killer (NK) cells, anti-nuclear antibody (ANA) and specific immune responses to autoantigens, cognitive function testing, brain imaging by single-positron emission computed tomography (SPECT), and magnetic resonance imaging (MRI) and related testing. Additional embodiments include use of stem cell lines wherein specific markers and properties are enhanced compared to native MSCs resulting in enhanced therapeutic benefits of the MSC-based therapy. We have treated 15 acute COVID-19 patients through compassionate use INDs (eINDs) authorized by FDA showing recovery from multiple organ failure including renal, hepatic, and nervous system failures.

There is evidence herein that MSC-based therapy can reverse virus-induced cellular pathology by regenerating affected cells, including pulmonary epithelial cells, by various regenerative effects including potent anti-inflammatory shifts of pro- to anti-inflammatory cytokines. The basis for the MSC therapy is related to COVID-19 disease etiology involving excessive pulmonary inflammation leading to acute respiratory distress where pre-clinical and clinical studies have shown significant remission of symptoms. The primary mechanism of therapeutic benefit, as described herein, is regenerative effects that repair organ and tissue damage caused by viral attacks on the lungs and other organs. Stem cells, especially MSCs, promote cell regeneration by reduction of inflammation, secretion of protective substances, transfer of mitochondria, anti-apoptosis/anti-oxidative effects, and modulation of the immune system. In addition, MSCs migrate to sites of inflammation through a system of chemokine signaling ligands and receptors. MSCs induce transformation of pro-inflammatory M1-Macrophages into anti-inflammatory M2-Macrophages as well as downregulation of the inflammatory cytokine signaling system by interference with IL-1 b activation of the cytokine-based inflammation cascade, thus providing powerful anti-inflammatory effects by both molecular and cellular modes of action. The detailed mechanism of action of MSC therapy in COVID-19 infections involves multiple clinical benefits including improved pulmonary, renal, and hepatic function, anti-coagulation effects, and viral clearance.

As opposed to vaccines, stem cell therapy provides alternative therapeutic benefits in cellular/organ system regeneration without being specific to just COVID-19 infections but rather to a broad range of viral infections that induce acute respiratory distress (ARDS), including SARS-CoV, SARS-CoV-2 (COVID-19), MERS-CoV and other known and unknown viruses. We have treated a Lupus patient, by an eIND, who had a severe adverse anaphylactic response to COVID-19 monoclonal antibodies that was resolved and who also experienced remission of Lupus symptoms. Stem cell therapy is a common procedure for the treatment of blood disorders including leukemia, lymphoma, and auto-immune conditions using transplantation of hematopoietic stem cells (HSCs). Other types of adult stem cells are now being transplanted to treat other conditions including skeletal muscular disorders, cardiovascular diseases, and cardiomyopathy. While activation of endogenous stem cells has been used to increase proliferation and differentiation of hematopoietic stem cells for several years, such activation of other adult stem cells including mesenchymal stem cells (MSCs), satellite cells, and neural stem cells (NSCs) has not yet reached routine clinical practice.

Adult mesenchymal stem cells (MSCs) are particularly relevant to the present disclosure. These cells were initially described by Arnold Caplan (Caplan, A I and Bruder, S P, Trends Mol Med 7: 259-264, 2001). MSCs may be derived from various tissues including bone marrow, adipose-tissue, dental pulp, umbilical cord, amniotic fluid and membranes, placenta, and other sources well-known to those skilled in the art (e.g., see US 2022/0389377, incorporated herein by reference). Comparative studies suggest differences in potency, differentiation capacity, growth rates and other cellular characteristics depending on the tissue source used to procure MSCs (e.g., see Example 17). This may be related to several factors, including medical status of the donor and environmental factors specific to the stem cell niche.

MSCs derived from bone marrow and adipose tissue have been subject to numerous clinical trials that provide preliminary evidence of safety and efficacy. A classic feature of MSCs is trilineage differentiation into adipocytes, chondrocytes, and osteoblasts, although multiple other cellular lineages may be derived from MSCs, including neural, kidney, and cardiac cells. Hence, MSCs have been extensively studied in skeletal muscular conditions such as osteoarthritis (OA). Several studies support safety and efficacy by intra-articular injections into knees, hips, and shoulder joints of OA patients. In addition, several other conditions may be treated by MSC transplants or transplants of progenitor cells derived from MSCs including stroke, myocardial infarct, and congestive heart failure.

Stem cell-based regenerative medicine is revolutionizing the treatment of medical conditions by treating underlying causes of injury and disease through rejuvenation and replacement of dead or injured cells and tissues. Thus, for example, the standard of care for treating osteoarthritis of joints is anti-inflammatory and pain management followed by prosthetic joint replacement. However, several clinical research groups (Lijima, H, et al, NPJ Regenerative Medicine (2018) 3:15) are providing clinical data showing that a single injection of MSCs into an arthritic joint relives pain, restores joint function, and induces the regeneration of cartilage thus obviating the need for a joint replacement. Additional clinical and pre-clinical studies show regenerative effects of MSCs and neural stem cells (NSCs) in various neurological conditions including stroke, Parkinson's disease, and other conditions characterized by death of particular types of neurons. Since these changes last for considerable periods of time, the stem cell regenerative effects may provide long-lived therapeutic benefits compared to the present standard of care.

### 1. Methods of Treating Post-Acute Sequelae of SARS CoV-2 (PASC)

Treatment embodiments include administering human MSCs, and in some cases, stem cells known as ALLORX STEM CELLS® to a patient in need. Cells are typically delivered via IV, intranasally, or intrathecally, although other known delivery routes are embodied. From about 1 to 2.5 million cells/kg body weight are delivered per treatment, particularly when the cells are administered by IV. Other number of cells can be administered, as long as the number provides a benefit to the patient. In another embodiment, the patient or the donor stem cells including MSCs, NSCs, Nephron progenitor cells, cardiomyocytes, and derivatives thereof are pretreated with a combination of curcumin and quercetin prior to administration of the cellular therapy to the patient. In this embodiment, the patient is treated until their serum concentration of each compound is approximately 500 nM or if the stem cells are pre-treated, the cell culture medium contains 500 nM of both curcumin and quercetin. Products that are secreted from stem cells have therapeutic value including exosomes, lipid bilayer enclosed vesicles of 30 to 150 nm or 80 nm to 200 nm and other soluble factors including proteins, RNA, microRNA and related biological molecules. Hence a further embodiment includes treatment of PASC with MSC derived exosomes, conditioned medium and specific biologic agents contained within the products secreted by MSCs at appropriate dosages to elicit symptom remission.

The methods of treating Post-Acute Sequelae of SARS CoV-2 (PASC) in a subject in need thereof utilize compositions that contain an effective amount of stem cells (e.g., mesenchymal stem cells or neural stem cells), isolated exosomes derived from the stem cells, cell culture medium containing the stem cell's secretome (e.g., stem cell secretome-conditioned cell culture medium), cell culture medium containing the stem cell's secretome but devoid of exosomes (e.g., exosome-depleted stem cell secretome-conditioned cell culture medium), a nutraceutical (e.g., resveratrol, curcumin, or quercetin) or some combination thereof.

A method of treating a subject with PASC may include administering to the subject an effective amount of (e.g., one or more of): (a) a plurality of stem cells, such as neural stem cells (NSCs) or mesenchymal stem cells (MSCs), e.g., umbilical cord-derived human mesenchymal stem cells (UC-MSCs), e.g., ALLORX STEM CELLS®; (b) a plurality of isolated exosomes of about 80-200 nanometers (nm) in diameter, which are derived from the stem cells (e.g., MSCs, UC-MSCs, or NSCs); (c) stem cell secretome-conditioned cell culture medium; (d) exosome-depleted stem cell secretome-conditioned cell culture medium; and/or (e) a nutraceutical, such as resveratrol, curcumin, and/or quercetin. With regards to compositions containing stem cells, the methods of treatment described herein may further include, as an option, increasing an mRNA and/or protein expression levels of sirtuin 1 (Sirt-1), C—X—C motif chemokine receptor 4 (CXCR4), heat shock protein 70 (HSP70), octamer-binding transcription factor 3/4 (Oct 3/4), fibroblast growth factor 2 (FGF-2), substance P, and/or fibroblast growth factor 21 (FGF-21), relative to unmodified stem cells. Further details on the methods of treatment and each of the administered compositions are described below.

### A. Administration

A composition containing about 5×10⁵ to 5×10⁶ (e.g., 5×10⁵ to 1×10⁶, 5×10⁵ to 1.5×10⁶, 1×10⁶ to 2.5×10⁶, 1.5×10⁶ to 3×10⁶, 2×10⁶ to 5×10⁶, or 4×10⁶ to 5×10⁶, e.g., 5×10⁵, 6×10⁵, 7×10⁵, 8×10⁵, 9×10⁵, 1×10⁶, 1.5×10⁶, 2×10⁶, 2.5×10⁶, 3×10⁶, 3.5×10⁶, 4×10⁶, 4.5×10⁶, or 5×10⁶) of the stem cells (e.g., MSCs, UC-MSCs, or NSCs) per kg of body weight may be administered to a subject with PASC. For example, a composition containing 1×10⁶ to 2.5×10⁶ (e.g., 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, or 2.5×10⁶) of the stem cells (e.g., MSCs, UC-MSCs, or NSCs) per body weight may be administered to the subject with PASC. In another example, a composition containing about 1×10⁶ (e.g., 9×10⁵, 1×10⁶, or 1.1×10⁶) of the stem cells (e.g., MSCs, UC-MSCs, or NSCs) per body weight is administered to the subject with PASC.

In yet another example a composition containing a total of about 5×10⁷ to 1×10⁸ (e.g., 5×10⁷ to 6×10⁷, 6×10⁷ to 7×10⁷, 7×10⁷ to 8×10⁷, 8×10⁷ to 9×10⁷, or 9×10⁷ to 1×10⁸, e.g., 5×10⁷, 6×10⁷, 7×10⁷, 8×10⁷, 9×10⁷, or 1×10⁸) of the stem cells (e.g., MSCs, UC-MSCs, or NSCs) may be administered to a subject with PASC. In particular, a composition containing a total of about 1×10⁸ (e.g., 9×10⁷, 9.1×10⁷, 9.2×10⁷, 9.3×10⁷, 9.4×10⁷, 9.5×10⁷, 9.6×10⁷, 9.7×10⁷, 9.8×10⁷, 9.9×10⁷, 1×10⁸, or 1.1×10⁸), of the stem cells (e.g., MSCs, UC-MSCs, or NSCs) may be administered to a subject with PASC.

Stem cells (e.g., MSCs, UC-MSCs, or NSCs) may be isolated from the subject to be treated for PASC or may be isolated from a healthy donor (e.g., a subject that does not have PASC). Thus, the stem cells (e.g., MSCs, UC-MSCs, or NSCs) may be allogenic or autologous.

By way of example, a composition containing UC-MSCs may include ALLORX STEM CELLS®, the manufacturing, harvesting, and quality control (QC) analysis of which is described in Examples 13 and 14. In brief, ALLORX STEM CELLS® are isolated (e.g., >95% purity, as determined by flow cytometry) MSCs from human umbilical cords and can repair or regenerate damaged tissues in a subject by differentiating into tissue-specific cells (e.g., adipocytes, chondrocytes, osteoblasts, and neural cells). ALLORX STEM CELLS® may express CD90, CD73, and CD105, as observed by flow cytometry (FC). ALLORX STEM CELLS® may not express CD11b, CD14, CD19, CD34, CD45, CD79a, CD126, and HLA-DR, as observed by FC. Further, ALLORX STEM CELLS® can secrete biological material, such as proteins (e.g., GM-CSF, MIP-3a, IL-6, IL-8, fractalkine, and MIP-1), lipids, and extracellular vesicles (e.g., exosomes). For example, ALLORX STEM CELLS® can secrete exosomes that are about 80 nm to about 200 nm or about 155 nm to about 175 nm (e.g., about 165 nm, on average) in diameter and may express CD9, CD63, CD81, CD44, CD29, and CD142, as observed by FC.

Compositions containing stem cells (e.g., MSCs, UC-MSCs, or NSCs) may further include a pharmaceutically acceptable carrier, excipient, or diluent and may not contain DMSO. Compositions containing stem cells may further include a cryopreservation medium, a basal medium, and/or a saline solution. The cryopreservation medium may be PRIME-XV® MSC FreeziS DMSO-Free medium (e.g., FUJIFILM Irvine Scientific Catalog number: 91140). The basal medium may be MCDB-131. Stem cells of the composition may be modified (e.g., genetically modified or pre-treated with a stem cell activator) such that they exhibit increased replication and/or migratory phenotypes relative to an unmodified stem cell (see section B below for more details).

Compositions containing stem cells (e.g., MSCs, UC-MSCs, or NSCs) may further include one or more stem cell activators selected from a histone deacetylase 1 (HDAC1) inhibitor (e.g., romidepsin (FK-228), vorinostat, romidepsin, belinostat, panobinostat, valproic acid (VPA), entinostat, curcumin, quercetin, and RG2833), a glycogen synthase kinase-3β (GSK-3B13) inhibitor (e.g., indirubin-3′-oxime, laduviglusib (CHIR-99821), and KY19382), a neurokinin-1 (NK-1) receptor agonist (e.g., substance P), a fibroblast growth factor (FGF) protein (e.g., FGF-2), a nutraceutical (e.g., resveratrol, curcumin, and quercetin), lithium (Li), and a neural cell adhesion molecule (NCAM) modulator (e.g., N-butylmannosamine) (see section B below for more details).

A composition containing a concentration of about 5×10⁹ to 5×10¹⁰ (e.g., 5×10⁹ to 1.5×10¹⁰, 1×10¹⁰ to 3×10¹⁰, 2×10¹⁰ to 4×10¹⁰, 3×10¹⁰ to 5×10¹⁰, 1×10¹⁰ to 5×10¹⁰, or 2.5×10¹⁰ to 5×10¹⁰, e.g., 5×10⁹, 6×10⁹, 7×10⁹, 8×10⁹, 9×10⁹, 1×10¹⁰, 1.5×10¹⁰, 2×10¹⁰, 2.5×10¹⁰, 3×10¹⁰, 3.5×10¹⁰, 4×10¹⁰, 4.5×10¹⁰, or 5×10¹⁰) of the isolated exosomes per mL may be administered to a subject with PASC. A composition containing about 100 μg to about 300 μg (e.g., 100 μg to 200 μg, 125 μg to 175 μg, or 200 μg to 300 μg, e.g., 100 μg, 125 μg, 150 μg, 175 μg, 200 μg, 225 μg, 250 μg, 275 μg, or 300 μg) of the isolated exosomes may be administered to a subject with PASC. The isolated exosomes may be derived from stem cell (e.g., MSC, UC-MSC, or NSC) secretome-conditioned cell culture medium. After removing exosomes from stem cell secretome-conditioned cell culture medium, exosomes may contain a volume (e.g., a volume of about 2.7×10⁻¹⁰ mm³ or less) of the cell culture medium from which they were isolated; this is still considered an “isolated” exosome.

By way of example, a composition containing isolated exosomes may include ALLOEX EXOSOMES®, the isolation of which is described in Example 15. In brief, ALLOEX EXOSOMES® are isolated (e.g., >80% purity (e.g., >85%, >90%, or >95% purity), as determined by flow cytometry) from UC-MSCs. ALLOEX EXOSOMES® are about 80 nm to about 200 nm or about 155 nm to about 175 nm (e.g., about 165 nm on average) in diameter and may express CD9, CD63, CD81, CD44, CD29, and CD142, as observed by FC.

A composition containing stem cell (e.g., MSC, UC-MSC, or NSC) secretome-conditioned cell culture medium may include about 100 pg/mL to 2000 pg/mL (e.g., 500 pg/mL to 1000 pg/mL, 800 pg/mL to 1600 pg/mL, or 1400 pg/mL to 2000 pg/mL, e.g., 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, 1000 pg/mL, 1050 pg/mL, 1100 pg/mL, 1150 pg/mL, 1200 pg/mL, 1250 pg/mL, 1300 pg/mL, 1350 pg/mL, 1400 pg/mL, 1450 pg/mL, 1500 pg/mL, 1550 pg/mL, 1600 pg/mL, 1650 pg/mL, 1700 pg/mL, 1750 pg/mL, 1800 pg/mL, 1850 pg/mL, 1900 pg/mL, 1950 pg/mL, 2000 pg/mL) of granulocyte-macrophage colony-stimulating factor (GM-CSF), macrophage inflammatory protein (MIP)-3 alpha (MIP-3a), interleukin (IL)-6, and IL-8 and may be administered to a subject having PASC. For example, a composition containing about 100 pg/mL to 1000 pg/mL (e.g., 100 pg/mL to 500 pg/mL, 250 pg/mL to 750 pg/mL, or 500 pg/mL to 1000 pg/mL, e.g., 100 pg/mL, 150 pg/mL, 200 pg/mL, 250 pg/mL, 300 pg/mL, 350 pg/mL, 400 pg/mL, 5450 pg/mL, 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, or 1000 pg/mL) of GM-CSF, MIP-3a, IL-6, and IL-8 in stem cell secretome-conditioned cell culture medium may be administered to a subject having PASC. The composition may further include about 10 pg/mL to 500 pg/mL (e.g., 10 pg/mL to 100 pg/mL, 10 pg/mL to 500 pg/mL, or 500 pg/mL to 1000 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, 250 pg/mL, 300 pg/mL, 350 pg/mL, 400 pg/mL, 450 pg/mL, or 500 pg/mL) of fractalkine and MIP-1 that is to be administered to the subject. For example, a composition containing stem cell secretome-conditioned cell culture medium may include about 100 pg/mL to 1000 pg/mL of GM-CSF, MIP-3a, IL-6, and IL-8, and about 10 pg/mL to about 100 pg/mL (e.g., 10 pg/mL to 50 pg/mL, 25 pg/mL to 75 pg/mL, or 50 pg/mL to 100 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, or 100 pg/mL) of fractalkine and MIP-1. These concentration ranges are envisioned per protein. The stem cells used to generate the stem cell secretome-conditioned cell culture medium may have been MSCs. The stem cells used to generate the stem cell secretome-conditioned cell culture medium may have been UC-MSCs (e.g., ALLORX STEM CELLS®). The stem cells used to generate the stem cell secretome-conditioned cell culture medium may have been NSCs.

A composition containing exosome-depleted stem cell secretome-conditioned cell culture medium may include about 100 pg/mL to 2000 pg/mL (e.g., 500 pg/mL to 1000 pg/mL, 800 pg/mL to 1600 pg/mL, or 1400 pg/mL to 2000 pg/mL, e.g., 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, 1000 pg/mL, 1050 pg/mL, 1100 pg/mL, 1150 pg/mL, 1200 pg/mL, 1250 pg/mL, 1300 pg/mL, 1350 pg/mL, 1400 pg/mL, 1450 pg/mL, 1500 pg/mL, 1550 pg/mL, 1600 pg/mL, 1650 pg/mL, 1700 pg/mL, 1750 pg/mL, 1800 pg/mL, 1850 pg/mL, 1900 pg/mL, 1950 pg/mL, 2000 pg/mL) of GM-CSF, MIP-3a, IL-6, and IL-8 and may be administered to a subject with PASC. For example, a composition containing about 100 pg/mL to 1000 pg/mL (e.g., 100 pg/mL to 500 pg/mL, 250 pg/mL to 750 pg/mL, or 500 pg/mL to 1000 pg/mL, e.g., 100 pg/mL, 150 pg/mL, 200 pg/mL, 250 pg/mL, 300 pg/mL, 350 pg/mL, 400 pg/mL, 5450 pg/mL, 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, or 1000 pg/mL) of GM-CSF, MIP-3a, IL-6, and IL-8 in exosome-depleted stem cell secretome-conditioned cell culture medium may be administered to a subject with PASC. The composition may further include about 10 pg/mL to 500 pg/mL (e.g., 10 pg/mL to 100 pg/mL, 10 pg/mL to 500 pg/mL, or 500 pg/mL to 1000 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, 250 pg/mL, 300 pg/mL, 350 pg/mL, 400 pg/mL, 450 pg/mL, or 500 pg/mL) of fractalkine and MIP-1 that is to be administered to the subject. For example, a composition containing exosome-depleted stem cell secretome-conditioned cell culture medium may include about 100 pg/mL to 1000 pg/mL of GM-CSF, MIP-3a, IL-6, and IL-8, and about 10 pg/mL to about 100 pg/mL (e.g., 10 pg/mL to 50 pg/mL, 25 pg/mL to 75 pg/mL, or 50 pg/mL to 100 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, or 100 pg/mL) of fractalkine and MIP-1. These concentration ranges are envisioned per protein. The stem cells used to generate the exosome-depleted stem cell secretome-conditioned cell culture medium may have been MSCs. The stem cells used to generate the exosome-depleted stem cell secretome-conditioned cell culture medium may have been UC-MSCs. The stem cells used to generate the exosome-depleted stem cell secretome-conditioned cell culture medium may have been NSCs.

A composition containing a nutraceutical (e.g., resveratrol, curcumin, and/or quercetin) may include about 50 nM to about 500 nM (e.g., about 50 nM to about 200 nM, about 100 nM, to about 300 nM, about 200 nM to about 400 nM, about 300 nM to about 500 nM, e.g., 50 nM, 60 nM, 70 nM, 80 nM, 90 nM, 100 nM, 125 nM, 150 nM, 175 nM, 200 nM, 225 nM, 250 nM, 275 nM, 300 nM, 325 nM, 350 nM, 375 nM, 400 nM, 425 nM, 450 nM, 475 nM, or 500 nM) of the nutraceutical and may be administered to a subject with PASC. For example, a composition containing a nutraceutical may include about 500 nM (e.g., 450 nM, 460 nM, 470 nM, 480 nM, 490 nM, 500 nM, 510 nM, 520 nM, 530 nM, 540 nM, or 550 nM) of resveratrol, curcumin, and/or quercetin. These concentration ranges are envisioned per nutraceutical.

Any of the compositions described herein (e.g., a composition containing a plurality of stem cells (e.g., MSCs, UC-MSCs, or NSCs), a plurality of isolated exosomes, stem cell (e.g., MSC, UC-MSC, or NSC) secretome-conditioned cell culture medium, exosome-depleted stem cell (e.g., MSC, UC-MSC, or NSC) secretome-conditioned cell culture medium, resveratrol, curcumin, quercetin, or some combination thereof) may be administered to a subject with PASC in a volume of about 0.5 milliliters (mL) to about 15 mL (e.g., 0.5 mL, 0.6 mL, 0.7 mL, 0.8 mL, 0.9 mL, 1 mL, 3 mL, 5 mL, 7 mL, 10 mL, 11 mL, 12 mL, 13 mL, 14 mL, or 15 mL), about 1 mL to about 10 mL (e.g., 1 mL, 2 mL, 3 mL, 4 mL, 5 mL, 6 mL, 7 mL, 8 mL, 9 mL, or 10 mL), or about 1 mL to about 6 mL, (e.g., 1 mL, 1.25 mL, 1.5 mL, 1.75 mL, 2 mL, 2.25 mL, 2.5 mL, 2.75 mL, 3 mL, 3.25 mL, 3.5 mL, 3.75 mL, 4 mL, 4.25 mL, 4.5 mL, 4.75 mL, 5 mL, 5.25 mL, 5.5 mL, 5.75 mL, or 6 mL). For example, the compositions described herein may be administered to a subject in a volume of about 10 mL (e.g., 9 mL, 10 mL, or 11 mL). The compositions described herein may also be added to a larger volume (e.g., 10 mL to 100 mL, such as 10 mL, 20 mL, 30 mL, 40 mL, 50 mL, 60 mL, 70 mL, 80 mL, 90 mL, or 100 mL) and slowly infused into the subject in need thereof.

Any of the compositions described herein (e.g., a composition containing a plurality of stem cells (e.g., MSCs, UC-MSCs, or NSCs), a plurality of isolated exosomes, stem cell (e.g., MSC, UC-MSC, or NSC) secretome-conditioned cell culture medium, exosome-depleted stem cell (e.g., MSC, UC-MSC, or NSC) secretome-conditioned cell culture medium, resveratrol, curcumin, quercetin, or some combination thereof) may be administered to a subject having PASC via a bolus (e.g., an IV bolus), a push (e.g., an IV push), or a drip (e.g., an IV drip). The compositions described herein may be administered to a subject over a period of 1 minute to 1 hour (e.g., 1 minute, 10 minutes, 20 minutes, 30 minutes, 40 minutes, 50 minutes, or 1 hour), 1 minute to 30 minutes (e.g., 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, or 30 minutes), or about 1 minute to about 10 minutes (e.g., 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, or 10 minutes).

The compositions described herein (e.g., a composition containing a plurality of stem cells (e.g., MSCs, UC-MSCs, or NSCs), a plurality of isolated exosomes, stem cell (e.g., MSC, UC-MSC, or NSC) secretome-conditioned cell culture medium, exosome-depleted stem cell (e.g., MSC, UC-MSC, or NSC) secretome-conditioned cell culture medium, resveratrol, curcumin, quercetin, or some combination thereof) may be administered to a subject with PASC at a frequency of once every one, two, three, four, five, six, seven, eight, nine, ten, eleven, or twelve months. For example, the compositions described herein may be administered to a subject at a frequency of once every three or six months (or as needed). In another example, the compositions described herein may be administered to a subject at a frequency of about once every three months (e.g., at a frequency of once every 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, or 99 days). In another example, the compositions described herein may be administered to a subject at a frequency of about once every six months (e.g., at a frequency of once every 170 days, 171 days, 172 days, 173 days, 174 days, 175 days, 176 days, 177 days, 178 days, 179 days, 180 days, 181 days, 182 days, 183 days, 184 days, 185 days, 186 days, 187 days, 188 days, 189 days or 190 days).

The compositions described herein (e.g., a composition containing a plurality of stem cells (e.g., MSCs, UC-MSCs, or NSCs), a plurality of isolated exosomes, stem cell (e.g., MSC, UC-MSC, or NSC) secretome-conditioned cell culture medium, exosome-depleted stem cell (e.g., MSC, UC-MSC, or NSC) secretome-conditioned cell culture medium, resveratrol, curcumin, quercetin, or some combination thereof) may be administered to a subject with PASC at a frequency of once every one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty days. For example, the compositions described herein may be administered to a subject at a frequency of once every six days. In another example, the compositions described herein may be administered to a subject at a frequency of once every seven days.

Administration of the compositions described herein may continue at any frequency described above for up to 1 week, 1 month, 6 months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, or more. Administration of the compositions described herein may continue at any frequency described above for the life of the subject. Administration of the compositions described herein may continue at any frequency described above until resolution of one or more symptoms of PASC is observed.

### B. Methods of Activating Stem Cells

Disclosed herein are methods for activating internal or transplanted stem cells, including methods for inducing stem cell proliferation, migration, and epigenetic reprogramming. Long COVID (PASC) can be treated in a subject in need thereof, the steps comprising administering nutraceuticals and combinations thereof. As used herein, nutraceuticals are defined as naturally occurring compounds that possess particular biological activities including, without limitation, epigenetic modulation, induction of stem cell migration, and/or stem cell proliferation.

In another embodiment in accordance with the present disclosure, compositions yielding desired levels of GSK3-3 and HDAC-I inhibition are provided. These compositions include, but are not limited to, curcumin as a GSK3-beta inhibitor and romidepsin (FK-228) as a potent and selective HDAC-I inhibitor. As shown in Example 8, curcumin also has epigenetic effects possibly through HDAC inhibition (Soflaei S S, et al, Cur Pharm Des. 2018; 24(2):123-129.) resulting in increased expression of Oct 3/4, Sirt-1, FGF-21, CXCR4, and Hsp70 that were comparable to the levels induced by VPA. Thus, the pleiotropic agent, curcumin, represents a single agent that is both a GSK3-beta inhibitor and also inhibits HDAC. An additional embodiment includes combinations of nutraceuticals to induce stem cell activation by administration to patients or exposure to cultured stem cells prior to transplantation into patients. The dosage is maintained by repeated administration to patients and when used to pre-treat MSCs, dosage includes maintenance of specified concentrations within the culture medium for at least the passage prior to MSC harvest. More specifically, curcumin and quercetin combine to activate stem cell migration at preferred dosages of 500 nM curcumin and 500 nM quercetin (Example 9). A further embodiment includes the combination of curcumin and resveratrol at the preferred concentrations of about 500 nM curcumin and about 500 nM resveratrol (Example 9).

Bioavailability of pharmacological and nutraceutical agents is an important component of therapy and thus liposomal formulations of curcumin and other formulations including piperine are used to maintain adequate bioavailability. The compositions of the present disclosure thus include various compounding processes well-known to those skilled in the art to alter pharmacokinetic properties and combine appropriate dosages of GSK3-beta inhibitor and HDAC inhibitors into single medications. Also, various administration routes may be utilized within the present disclosure to optimize drug delivery to CNS. Thus, for example, but without limitation, activating drugs and NCAM modulating substances may be compounded together or alone into nasal sprays, eye drops, appropriate formulations for intrathecal delivery, dermal patches, etc. These formulations may involve the appropriate use of nano technology, liposomes; emulsions, ointments, etc. as are well-known to those skilled in the art.

Stem cell activation does not necessarily involve a chemical process but may also occur through the use of appropriate energy input into stem cells, such as low-power laser activation, exposure to electrical/magnetic fields or light or sound at specific intensities and frequencies.

Gene expression profiling also provides detailed analysis of the epigenetic reprogramming resulting from epigenetic modulation as by HDAC inhibition and the subsequent alterations in DNA methylation. This same approach may be extended to other patient-specific cellular samples besides NSCs derived from nasal epithelium biopsies including, without limitation, mononuclear cells contained in whole blood.

In a further embodiment the treated cells have specific properties that are significantly distinct from native, untreated MSCs including epigenetic effects whereby: a) expression of Sirt-1 is increased by 200 to 300-fold, b) expression of CXCR4 is increased by 10 to 20-fold, c) heat shock protein 70 (Hsp70) expression levels increase by up to 20-fold, d) Oct 3/4 expression is increased by approximately 10 to 20-fold, and e) expression of FGF-21 is increased by 10 to 25-fold in the treated cell. The rate of MSC proliferation and migration is also increased, the latter is due to increased activity of CXCR-4 and MMP-9 (e.g., see Examples 1-10). In this embodiment, the epigenetically modified MSCs are administered to a patient in need at about 1 to 2.5 million cells/kg. As above, and for each MSC administration embodiment herein, the cells can be administered more than once, more than twice, more than three times, over the course of days, months, or years to a subject. In addition, combinations of MSCs can be used, for example, untreated MSCs, nutraceutical treated MSCs, epigenetically modified MSCs, and the like, can be used on the same subject at the same or different times.

The methods described herein may further include steps of activating (e.g., increasing migration and/or proliferation phenotypes) transplanted stem cells (e.g., MSCs, UC-MSCs, or NSCs) or endogenous stem cells (e.g., NSCs). For stem cell activation, the methods may include genetically engineering the stem cells by transient or constitutive transfection to express exogenous substance P and/or FGF-2. For stem cell activation, the methods may include, or further include, pre-treating the stem cells with a stem cell activating agent. A stem cell activating agent may be, for example, one or more HDAC1 inhibitors that are known to enhance function of stem cells through increased migration, proliferation, and upregulation of Sirt-I, Oct3/4, CXCR4, and HSP70 (see, e.g., US 2022/0389377 and Hennig, K M, et al, Mol Neuropsychiatry 2017; 3: 53-71, each of which is incorporated herein by reference). Further examples of activating stem cells are provided below.

i. Genetic Manipulation of Stem Cells

The methods of treating a subject with PASC described herein may utilize a plurality of isolated stem cells (e.g., MSCs, UC-MSCs, or NSCs, such as ALLORX STEM CELLS®) that are genetically modified to increase migration and proliferation phenotypes.

Stem cells (e.g., MSCs, UC-MSCs, or NSCs) may be isolated from the subject with PASC or may be isolated from a healthy donor (e.g., a subject that does not have PASC). Stem cells (e.g., MSCs, UC-MSCs, or NSCs) may be modified (e.g., modified stem cells, e.g., modified MSCs, modified UC-MSCs, or modified NSCs) as described herein. For example, stem cells (e.g., MSCs, UC-MSCs, or NSCs) may be genetically modified to express one, two, three, four, five, or more copies of FGF-2 and/or substance P (e.g., as mRNA and/or protein). Upon transfection, the modified stem cells may have an increased expression of Sirt-1, CXCR4, HSP70, Oct 3/4, and/or FGF-21 mRNA and/or protein levels relative to unmodified stem cells.

Stem cells (e.g., MSCs, UC-MSCs, or NSCs) may be modified by introducing exogenous genetic material (e.g., DNA, cDNA, RNA, or mRNA) into the stem cell. For example, a nucleic acid vector, plasmid, circular RNA, or mRNA molecule may be introduced into the stem cells, e.g., by way of transfection (e.g., microinjection, optical transfection, biolistic transfection, electroporation, nucleofection, lipofection, sonoporation, and magnetofection). The exogenous genetic material (e.g., nucleic acid vector, plasmid, circular RNA, or mRNA molecule) may contain one or more (e.g., 1, 2, 3, 4, 5, 6, 7, 8, 9, 10 or more) nucleotide sequences encoding one or more copies of the following genes: FGF-2 and/or substance P. This expression can augment the stem cells proliferation and migration phenotypes, thereby causing the stem cell to be activated.

Exemplary genes, mRNA transcripts, and protein sequences to be expressed in the stem cells are provided in Table 1 below.

TABLE 1
Exemplary Expression

| Name | NCBI Accession Numbers | Sequence Type |
|---|---|---|
| Substance P | Nucleotides 291-323 of | Transcript Sequence |
| | NM_003182.3 | |
| | Amino acid residues 58-68 of | Protein Sequence |
| | NP_003173.1 | |
| Fibroblast growth factor 2 (FGF-2) | Gene ID: 2247 | Genomic Sequence |
| | NM_002006.6 | Transcript Sequence |
| | NM_001361665.2 | |
| | NP_001997.5 | Protein Sequence |
| Sirtuin 1(Sirt-1) | Gene ID: 23411 | Genomic Sequence |
| | NM_012238.5 | Transcript Sequence |
| | NM_001142498.2 | |
| | NM_001314049.2 | |
| | NP_036370.2 | Protein Sequence |
| | NP_001135970.1 | |
| | NP_001300978.1 | |
| C-X-C motif chemokine receptor 4 | Gene ID: 7852 | Genomic Sequence |
| (CXCR4) | NM_003467.3 | Transcript Sequence |
| | NM_001348059.2 | |
| | NM_001348056.2 | |
| | NM_001348060.2 | |
| | XM_047445802.1 | |
| | NM_001008540.2 | |
| | XM_054343837.1 | |
| | NP_003458.1 | Protein Sequence |
| | NP_001334988.1 | |
| | NP_001334985.1 | |
| | NP_001334989.1 | |
| | XP_047301758.1 | |
| | NP_001008540.1 | |
| | XP_054199812.1 | |
| Heat shock protein 70 (HSP70) | Gene ID: 3308 | Genomic Sequence |
| member 4 | NM_002154.4 | Transcript Sequence |
| | XM_054352493.1 | |
| | NP_002145.3 | Protein Sequence |
| | XP_054208468.1 | |
| Heat shock protein 70 (HSP70) | Gene ID: 3303 | Genomic Sequence |
| member 1A | NM_005345.6 | Transcript Sequence |
| | NP_005336.3 | Protein Sequence |
| Octamer-binding transcription factor | Gene ID: 5460 | Genomic Sequence |
| 3/4 (Oct 3/4) | NM_002701.6 | Transcript Sequence |
| | NM_001173531.3 | |
| | NM_203289.6 | |
| | NM_001285986.2 | |
| | NM_001285987.1 | |
| | NP_002692.2 | Protein Sequence |
| | NP_001167002.1 | |
| | NP_976034.4 | |
| | NP_001272915.1 | |
| | NP_001272916.1 | |
| Fibroblast growth factor 21 (FGF-21) | Gene ID: 26291 | Genomic Sequence |
| | NM_019113.4 | Transcript Sequence |
| | NP_061986.1 | Protein Sequence |
| | NP_001348594.1 | |
| | NP_001348594.1 | |

ii. In Vitro Chemical Induction of Stem Cell Activation

Prior to the administration of the composition, stem cells (including modified stem cells, e.g., genetically modified stem cells) may be pre-treated with one or more stem cell activating agents. Stem cell activating agents can stimulate stem cell migration and/or proliferation. Stem cell activators include, but are not limited to, a HDAC1 inhibitor (e.g., romidepsin (FK-228), vorinostat, romidepsin, belinostat, panobinostat, VPA, entinostat, curcumin, quercetin, and RG2833), a GSK-3B3 inhibitor (e.g., indirubin-3′-oxime, laduviglusib (CHIR-99821), and KY19382), a NK-1 receptor agonist (e.g., substance P), a FGF protein (e.g., FGF-2), a nutraceutical (e.g., resveratrol, curcumin, and quercetin), and Li (e.g., lithium chloride).

The stem cells (e.g., MSCs, UC-MSCs, or NSCs) may be pre-treated by contacting a plurality of stem cells with about 1 nanomolar (nM) to about 10 micromolar (μM) of an HDAC1 inhibitor (e.g., romidepsin (FK-228), vorinostat, romidepsin, belinostat, panobinostat, VPA, entinostat, curcumin, quercetin, and RG2833) (e.g., 1 nM, 50 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or 10 μM) about 100 nM to about 1 μM (e.g., 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, or 1 μM), about 400 nM to about 600 nM (e.g., 400 nM, 425 nM, 450 nM, 475 nM, 500 nM, 525 nM, 550 nM, 575 nM, or 600 nM), or about 500 nM (e.g., 450 nM, 460 nM, 470 nM, 480 nM, 490 nM, 500 nM, 510 nM, 520 nM, 530 nM, 540 nM, or 550 nM of an HDAC1 inhibitor (e.g., romidepsin (FK-228), vorinostat, romidepsin, belinostat, panobinostat, VPA, entinostat, curcumin, quercetin, and RG2833). For example, the plurality of UC-MSCs may be contacted with about 500 nM of curcumin.

The stem cells (e.g., MSCs, UC-MSCs, or NSCs) may be pre-treated by contacting a plurality of stem cells with about 1 nM to about 10 μM of a GSK-3B3 inhibitor (e.g., indirubin-3′-oxime, laduviglusib (CHIR-99821), and KY19382) (e.g., 1 nM, 50 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or 10 μM) about 100 nM to about 1 μM (e.g., 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, or 1 μM), about 400 nM to about 600 nM (e.g., 400 nM, 425 nM, 450 nM, 475 nM, 500 nM, 525 nM, 550 nM, 575 nM, or 600 nM), or about 500 nM (e.g., 450 nM, 460 nM, 470 nM, 480 nM, 490 nM, 500 nM, 510 nM, 520 nM, 530 nM, 540 nM, or 550 nM of a GSK-3B3 inhibitor (e.g., indirubin-3′-oxime, laduviglusib (CHIR-99821), and KY19382)).

The stem cells (e.g., MSCs, UC-MSCs, or NSCs) may be pre-treated by contacting a plurality of stem cells with about 1 nM to about 5 nM of a NK-1 receptor agonist (e.g., substance P) (e.g., about 1 nM to about 4 nM, about 1 nM to about 3 nM, or about 2 nM to about 3 nM, e.g., 1 nM, 1.5 nM, 2 nM, 2.1 nM, 2.2 nM, 2.3 nM, 2.4 nM, 2.5 nM, 2.6 nM, 2.7 nM, 2.8 nM, 2.9 nM, 3 nM, 3.5 nM, 4 nM, 4.5 nM, or 5 nM of a NK-1 receptor agonist (e.g., substance P)). For example, the plurality of UC-MSCs may be contacted with about 2.5 nM of substance P.

The stem cells (e.g., MSCs, UC-MSCs, or NSCs) may be pre-treated by contacting a plurality of stem cells with an FGF protein (e.g., FGF-2). The FGF protein may be provided in a final concentration of about 3 ng/mL to about 10 ng/mL (e.g., about 3 ng/mL to about 6 ng/mL or about 3.5 ng/mL to about 4.5 ng/mL, e.g., 3 ng/mL, 3.5 ng/mL, 3.6 ng/mL, 3.7 ng/mL, 3.8 ng/mL, 3.9 ng/mL, 4 ng/mL, 4.1 ng/mL, 4.2 ng/mL, 4.3 ng/mL, 4.4 ng/mL, 4.5 ng/mL, 5 ng/mL, 6 ng/mL, 7 ng/mL, 8 ng/mL, 9 ng/mL, or 10 ng/mL). For example, the plurality of UC-MSCs may be contacted with about 4.2 ng/mL of FGF-2.

The stem cells (e.g., MSCs, UC-MSCs, or NSCs) may be pre-treated by contacting a plurality of stem cells with a nutraceutical (e.g., resveratrol, curcumin, or quercetin) at a concentration of about 1 nM to about 10 μM (e.g., 1 nM, 50 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or 10 μM) about 100 nM to about 1 μM (e.g., 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, or 1 μM), about 400 nM to about 600 nM (e.g., 400 nM, 425 nM, 450 nM, 475 nM, 500 nM, 525 nM, 550 nM, 575 nM, or 600 nM), or about 500 nM (e.g., 450 nM, 460 nM, 470 nM, 480 nM, 490 nM, 500 nM, 510 nM, 520 nM, 530 nM, 540 nM, or 550 nM of a nutraceutical (e.g., resveratrol, curcumin, or quercetin)). For example, the plurality of UC-MSCs may be contacted with about 253 nM of resveratrol.

The stem cells (e.g., MSCs, UC-MSCs, or NSCs) may be pre-treated by contacting a plurality of stem cells with Li (e.g., lithium chloride) at a concentration of about 10 μM to about 200 μM (e.g., about 10 μM, 25 μM, 50 μM, 75 μM, 100 μM, 125 μM, 150 μM, 175 μM, or 200 μM), about 50 μM to about 125 μM (e.g., about 50 μM, 70 μM, 90 μM, 110 μM, or 125 μM), about 75 μM to about 85 μM (e.g., about 75 μM, 76 μM, 77 μM, 78 μM, 79 μM, 80 μM, 81 μM, 82 μM, 83 μM, 84 μM, or 85 μM), or about 79 μM (e.g., 71.1 μM, 72 μM, 73 μM, 74 μM, 75 μM, 76 μM, 77 μM, 78 μM, 78.5 μM, 79 μM, 79.5 μM, 80 μM, 81 μM, 82 μM, 83 μM, 84 μM, 85 μM, 86 μM, or 86.9 μM of Li (e.g., lithium chloride)). For example, the plurality of UC-MSCs may be contacted with about 79 μM of Li.

By way of example, the stem cells (e.g., MSCs, UC-MSCs, or NSCs) may be pre-treated by contacting the plurality of stem cells with curcumin at a concentration of about 0.1 μM to about 1 μM (e.g., about 0.1 μM to about 0.5 μM, about 0.3 μM to about 0.7 μM, or about 0.5 μM to about 1 μM, e.g., 100 nM, 200 nM, 300 nM, 400 nM, 500 nM, 600 nM, 700 nM, 800 nM, 900 nM, or 1000 nM of curcumin).

In another example, the stem cells (e.g., MSCs, UC-MSCs, or NSCs) may be concomitantly pre-treated with two HDAC1 inhibitors, such as by contacting the plurality of stem cells with curcumin at a concentration of about 0.1 μM to about 1 μM (e.g., about 0.1 μM to about 0.5 μM, about 0.3 μM to about 0.7 μM, or about 0.5 μM to about 1 μM, e.g., 100 nM, 200 nM, 300 nM, 400 nM, 500 nM, 600 nM, 700 nM, 800 nM, 900 nM, or 1000 nM) and quercetin at a concentration of about 0.1 μM to about 1 μM (e.g., about 0.1 μM to about 0.5 μM, about 0.3 μM to about 0.7 μM, or about 0.5 μM to about 1 μM, e.g., 100 nM, 200 nM, 300 nM, 310 nM, 317 nM, 325 nM, 350 nM, 375 nM, 400 nM, 500 nM, 600 nM, 700 nM, 800 nM, 900 nM, or 1000 nM).

In yet another example, the stem cells (e.g., MSCs, UC-MSCs, or NSCs) may be pre-treated by contacting the plurality of stem cells with VPA at a concentration of about 20 μM to about 100 μM (e.g., about 20 μM to about 50 μM, about 35 μM to about 45 μM, about 40 μM to about 70 PM, about 60 μM to about 80 μM, or about 80 μM to about 100 μM, e.g., 20 μM, 30 μM, 31 μM, 32 μM, 33 μM, 34 μM, 35 μM, 36 μM, 37 μM, 38 μM, 39 μM, 40 μM, 50 μM, 60 μM, 70 μM, 80 μM, 90 μM, or 100 μM).

In yet another example, the stem cells (e.g., MSCs, UC-MSCs, or NSCs) may be pre-treated by contacting the plurality of stem cells with Li at a concentration of about 10 μM to about 200 μM (e.g., about 10 μM to about 40 μM, about 30 μM to about 70 μM, about 75 μM to about 85 μM, about 50 μM to about 100 μM, about 75 μM to about 150 μM, or about 100 μM to about 200 μM, e.g., 10 μM, 20 μM, 30 μM, 40 μM, 50 μM, 60 μM, 70 μM, 71 μM, 72 μM, 73 μM, 74 μM, 75 μM, 76 μM, 77 μM, 78 μM, 79 μM, 80 μM, 81 μM, 82 μM, 83 μM, 84 μM, 85 μM, 86 μM, 87 μM, 88 μM, 89 μM, 90 μM, 100 μM, 110 μM, 120 μM, 130 μM, 140 μM, 150 μM, 160 μM, 170 μM, 180 μM, 190 PM, or 200 PM).

In yet another example, the stem cells (e.g., MSCs, UC-MSCs, or NSCs) may be concomitantly pre-treated with an HDAC inhibitor and Li, such as by contacting the plurality of stem cells with VPA at a concentration of about 10 μM to about 125 μM (e.g., about 10 μM to about 40 PM, about 35 μM to about 45 μM, about 25 μM to about 75 μM, about 50 μM to about 100 PM, or about 75 μM to about 125 μM, e.g., 10 μM, 20 μM, 30 μM, 31 μM, 32 μM, 33 μM, 34 μM, 35 μM, 36 μM, 37 μM, 38 μM, 39 μM, 40 μM, 50 μM, 60 μM, 70 μM, 80 μM, 90 μM, 100 μM, 110 μM, 120 μM, or 125 μM) and Li at a concentration of about 200 μM (e.g., about 180 μM to about 220 μM or about 190 μM to about 210 μM, e.g., 180 μM, 185 μM, 190 μM, 195 μM, 200 μM, 205 μM, 210 μM, 215 μM, or 220 μM).

In yet another example, the stem cells (e.g., MSCs, UC-MSCs, or NSCs) may be concomitantly pre-treated with an HDAC1 inhibitor and a nutraceutical, such as by contacting the plurality of stem cells with VPA at a concentration of about 20 μM to about 250 μM (e.g., about 20 μM to about 50 μM, about 25 μM to about 75 μM, about 50 μM to about 100 μM, about 150 μM to about 200 μM, or about 175 μM to about 250 μM, e.g., 20 μM, 40 μM, 60 μM, 80 μM, 100 μM, 125 μM, 150 μM, 175 μM, 200 μM, 225 μM, or 250 μM) and curcumin at a concentration of about 100 nM to about 1 μM (e.g., about 100 nM to about 500 nM, about 250 nM to about 750 nM, or about 500 nM to about 1 μM, e.g., 100 nM, 200 nM, 300 nM, 400 nM, 500 nM, 600 nM, 700 nM, 800 nM, 900 nM, or 1000 nM).

In yet another example, the stem cells (e.g., MSCs, UC-MSCs, or NSCs) may be pre-treated by contacting of the plurality of stem cells is with substance P at a concentration of about 1 nM to about 5 nM (e.g., about 1 nM to about 4 nM, about 1 nM to about 3 nM, or about 2 nM to about 3 nM, e.g., 1 nM, 1.5 nM, 2 nM, 2.1 nM, 2.2 nM, 2.3 nM, 2.4 nM, 2.5 nM, 2.6 nM, 2.7 nM, 2.8 nM, 2.9 nM, 3 nM, 3.5 nM, 4 nM, 4.5 nM, or 5 nM of substance P).

In yet another example, stem cells (e.g., MSCs, UC-MSCs, or NSCs) may be pre-treated by contacting a plurality of stem cells with an FGF-2 protein. The FGF-2 protein may be provided in a final concentration of about 3 ng/mL to about 10 ng/mL (e.g., about 3 ng/mL to about 6 ng/mL or about 3.5 ng/mL to about 4.5 ng/mL, e.g., 3 ng/mL, 3.5 ng/mL, 3.6 ng/mL, 3.7 ng/mL, 3.8 ng/mL, 3.9 ng/mL, 4 ng/mL, 4.1 ng/mL, 4.2 ng/mL, 4.3 ng/mL, 4.4 ng/mL, 4.5 ng/mL, 5 ng/mL, 6 ng/mL, 7 ng/mL, 8 ng/mL, 9 ng/mL, or 10 ng/mL of FGF-2 protein).

In yet another example, the stem cells (e.g., MSCs, UC-MSCs, or NSCs) may be concomitantly pre-treated with two or more nutraceuticals, such as by contacting of the plurality of stem cells is with curcumin at a concentration of about 500 nM (e.g., about 450 nM to about 550 nM or about 475 nM to about 525 nM, e.g., 450 nM, 460 nM, 470 nM, 480 nM, 490 nM, 500 nM, 510 nM, 520 nM, 530 nM, 540 nM, or 550 nM) and resveratrol at a concentration of about 50 nM to about 500 nM (e.g., about 50 nM to about 100 nM, about 75 nM to about 150 nM, about 100 nM to about 200 nM, about 150 nM to about 300 nM, about 200 nM, to about 400 nM, about 240 nM to about 260 nM, about 300 nM to about 500 nM, or about 400 nM to about 500 nM, e.g., 50 nM, 100 nM, 150 nM, 200 nM, 250 nM, 253 nM, 275 nM, 300 nM, 350 nM, 400 nM, 450 nM, or 500 nM).

Prior to the administration of the composition, pre-treatment of the stem cells (e.g., MSCs, UC-MSCs, or NSCs) may include contacting the stem cells with one, two, three, four, five, or more of the stem cell activators described above (e.g., romidepsin (FK-228), vorinostat, romidepsin, belinostat, panobinostat, VPA, entinostat, curcumin, quercetin, RG2833, indirubin-3′-oxime, laduviglusib (CHIR-99821), and KY19382, substance P, FGF-2, resveratrol, curcumin, and quercetin).

Contacting the stem cells (e.g., MSCs, UC-MSCs, or NSCs) with the stem cell activators described above (e.g., the HDAC1 inhibitors, GSK-3B13 inhibitors, NK-1 receptor agonist, FGF proteins, nutraceuticals, and Li) may occur for about 1 day to about 6 weeks (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days), about 1-3 weeks (e.g., 1 week, 10 days, 11 days, 2 weeks, 17 days, 18 days, 3 weeks), about 2 weeks (e.g., 13 days, 14 days, or 15 days), about 1 week, about 6 days, about 5 days, about 4 days, about 3 days, about 2 days, or for about 1 day.

Upon contacting the stem cells (e.g., MSCs, UC-MSCs, or NSCs) with one or more of the stem cell activators described above (e.g., the HDAC1 inhibitors, GSK-3B13 inhibitors, NK-1 receptor agonist, FGF proteins, nutraceuticals, and Li) proliferation and/or migration of the stem cells may be increased relative to stem cells that are not in contact with the stem cell activating agent. Standard laboratory techniques can be used to assess activation of stem cell proliferation (e.g., cell counts and 5-Bromo-2′-Deoxyuridine (BrdU) uptake assays) and migration (e.g., scratch and transwell migration assays).

Stem cell activation may be accompanied by an increased expression level of Sirt-1, CXCR4, HSP70, Oct 3/4, and/or FGF-21, as described herein. Upon contacting the stem cells (e.g., MSCs, UC-MSCs, or NSCs) with one or more of the stem cell activators described above (e.g., the HDAC1 inhibitors, GSK-3B13 inhibitors, NK-1 receptor agonist, FGF proteins, nutraceuticals, and Li) the mRNA and/or protein expression levels of Sirt-1, CXCR4, HSP70, Oct 3/4, and/or FGF-21 may increase in the stem cells. For example, the expression level of Sirt-1 may increase in the stem cells by about 200-fold to about 300-fold; the expression level of CXCR4 may increase in the stem cells by about 10-fold to about 20-fold; the expression level of HSP70 may increase in the stem cells by about 2-fold to about 20-fold; the expression level of Oct 3/4 may increase in the stem cells by about 10-fold to about 20-fold; and/or the expression level of FGF-21 may increase in the stem cells by about 5-fold to about 25-fold, relative to stem cells that are not in contact with the stem cell activating agent(s). Standard laboratory techniques can be used to assess mRNA expression levels (e.g., PCR, RT-PCR, qPCR, RT-qPCR, microarray analysis, Northern blot, MASSARRAY® technique, SAGE, RNA-sequencing) and protein expression levels (e.g., FC, fluorescence-activated cell sorting (FACS) Western blot, enzyme-linked immunosorbent assay (ELISA), mass spectrometry (MS), immunofluorescence (IF), immunoprecipitation (IP), radioimmunoassay, dot blotting, high performance liquid chromatography (HPLC), surface plasmon resonance, optical spectroscopy, and immunohistochemistry (IHC). Exemplary transcript and protein sequences that may increase in a stem cell after stem cell activation are provided in Table 2 below.

TABLE 2
Exemplary Sequences that Increase in Stem Cells Upon Stem Cell Activation

| Name | NCBI Accession Numbers | Sequence Type |
|---|---|---|
| Sirtuin 1(Sirt-1) | Gene ID: 23411 | Genomic Sequence |
| | NM_012238.5 | Transcript Sequence |
| | NM_001142498.2 | |
| | NM_001314049.2 | |
| | NP_036370.2 | Protein Sequence |
| | NP_001135970.1 | |
| | NP_001300978.1 | |
| C-X-C motif chemokine receptor 4 | Gene ID: 7852 | Genomic Sequence |
| (CXCR4) | NM_003467.3 | Transcript Sequence |
| | NM_001348059.2 | |
| | NM_001348056.2 | |
| | NM_001348060.2 | |
| | XM_047445802.1 | |
| | NM_001008540.2 | |
| | XM_054343837.1 | |
| | NP_003458.1 | Protein Sequence |
| | NP_001334988.1 | |
| | NP_001334985.1 | |
| | NP_001334989.1 | |
| | XP_047301758.1 | |
| | NP_001008540.1 | |
| | XP_054199812.1 | |
| Heat shock protein 70 (HSP70) | Gene ID: 3308 | Genomic Sequence |
| member 4 | NM_002154.4 | Transcript Sequence |
| | XM_054352493.1 | |
| | NP_002145.3 | Protein Sequence |
| | XP_054208468.1 | |
| Heat shock protein 70 (HSP70) | Gene ID: 3303 | Genomic Sequence |
| member 1A | NM_005345.6 | Transcript Sequence |
| | NP_005336.3 | Protein Sequence |
| Octamer-binding transcription factor | Gene ID: 5460 | Genomic Sequence |
| 3/4 (Oct 3/4) | NM_002701.6 | Transcript Sequence |
| | NM_001173531.3 | |
| | NM_203289.6 | |
| | NM_001285986.2 | |
| | NM_001285987.1 | |
| | NP_002692.2 | Protein Sequence |
| | NP_001167002.1 | |
| | NP_976034.4 | |
| | NP_001272915.1 | |
| | NP_001272916.1 | |
| Fibroblast growth factor 21 (FGF-21) | Gene ID: 26291 | Genomic Sequence |
| | NM_019113.4 | Transcript Sequence |
| | NP_061986.1 | Protein Sequence |
| | NP_001348594.1 | Protein Sequence |

iii. In Vivo Chemical Induction of Stem Cell Activation

Adult stem cells reside in various regions throughout the body of warm-blooded animals including bone marrow where both hematopoietic and mesenchymal stem cells reside, satellite cells within muscles, various MSCs within other tissues, e.g., teeth pulp, endocrine glands, arteries (as pericytes). Within the nervous system, neuronal stem cells (NSCs) are concentrated within the subventricular zone (SVZ) of the lateral ventricles, the dentate gyrus of the hippocampus, within the olfactory epithelium and diffusely within the frontal cortex. The rostral migratory stream (RMS) is a Constructures of continuous flow of neural progenitor cells, neural stem cells and transiently activated neural progenitor cells whereby a migratory stream of cells provide potential regeneration of the adult brain through cellular activation at the level of the SVZ in the lateral ventricles.

While hematopoietic stem cells (HSCs) are relatively active and responsible for production of 200 billion red blood cells daily, other niche regions of adult stem cells are typically quiescent and are activated by a specific stimulation, such as response to injury. Stem cell activation, as referred to herein, involves three separate, although not necessarily independent, biological processes: proliferation, migration, and epigenetic reprogramming. Proliferation increases the number of stem cells through cell division, while migration is characterized by movement to specific targets mediated through chemokine/chemokine receptor systems within stem cells and the local environment. This migration of stem cells allows for various regenerative and repair processes to occur at sites remote from the actual stem cell niches within the body. Epigenetic reprogramming involves increased expression of specific genes involved with stem cell pluripotency, e.g., Oct 3/4 (Octamer-binding transcription factor 3/4) and longevity (Sartain family including Sirtuin-1 or SIRT-1) and certain members of the fibroblast growth factor (FGF) family of proteins, including FGF-21. Epigenetic reprogramming is mediated through HDAC inhibition resulting in modulation of DNA methylation patterns yielding altered gene expression including increased or decreased expression of specific genes. Proliferation is induced by several molecular mechanisms that yield increased numbers of cells through the process of cell division. Other stem cell activating agents are also disclosed herein, including specific agents that provide potent and selective inhibition of GSK3-β (glycogen synthetase 3-β) and HDAC-I (histone deacetylase I).

Another embodiment refers to concomitant treatments to both activate neural stem cells and to enhance the migration beyond the RMS to other brain regions in a patient having or suspected of having PASC. The brain fog and other neurological symptoms of Long COVID may be treated through these cellular mechanisms. Neural stem cells are derived primarily from cell division in the SVZ of the lateral ventricle and then migrate through the RMS eventually differentiating into neurons within the olfactory bulb. Injury, cell degeneration, infection, etc. result in collateral NSC migration to these sites from the RMS. This signaling is mediated through various factors elaborated at the injured site that promote tropic movement of NSCs away from the RMS. The RMS is composed of complex network of cells, vasculature and extracellular matrix molecules (ECM) that usually limit cell movement to the RMS itself. Specific ECM molecules, especially neural cell adhesion molecule (NCAM) and Tenascin R, play significant roles in the migration of NSCs within the RMS. NCAM is particularly important in controlling RMS cellular migration and it interacts with itself or other molecules to regulate cellular migration within the RMS. The addition of homopolymers of sialic acid (polysialic acid or PSA) is a highly regulated post-translational modification of the NCAM. Two enzymes, the polysialyltransferases, ST8Sial I (ST8Sial) and ST8Sial V (ST8SiaV) are responsible for the biosynthesis of PSA. Since the removal of PSA from NCAM is known to allow diffuse migration of NSC and related cells from the RMS to surrounding CNS regions (Battista and Tunisair, J. Neurosis 30:3995-4003, 2010) the present disclosure includes concomitant methods to activate NSCs within the brain and particularly in the SVZ of the lateral ventricles together with methods to modify and limit the restraints from ectopic migration by interference with the process of PSA addition to NCAM. For example, and without limitation, this may include use of synthetic precursors including N-butylmannosamine, small inhibiting RNA to ST8Sial, ST8SiaV, peptide mimetics that block addition of PSA to NCAM, and other enzymatic inhibitors and negative regulators of expression that result in blocking the addition of PSA to NCAM.

In the methods of treating a subject with PASC, the method may further include treating the subject with a stem cell activator prior to administration of a composition containing stem cells (e.g., MSCs, UC-MSCs, or NSCs). In this embodiment, the subject is pre-treated with one or more stem cell activators until their serum concentration of each compound reaches a concentration effective enough to stimulate activation of the transplanted stem cells (e.g., exogenous MSCs, UC-MSCs, or NSCs) and/or endogenous stem cells (e.g., the NSCs residing in the subject's brain). Stem cell activators include, but are not limited to, an HDAC1 inhibitor (e.g., romidepsin (FK-228), vorinostat, romidepsin, belinostat, panobinostat, VPA, entinostat, curcumin, quercetin, and RG2833), a GSK-3B13 inhibitor (e.g., indirubin-3′-oxime, laduviglusib (CHIR-99821), and KY19382), a NK-1 receptor agonist (e.g., substance P), a FGF protein (e.g., FGF-2), a nutraceutical (e.g., resveratrol, curcumin, and quercetin), Li (e.g., lithium chloride), and an NCAM modulator. Exemplary NCAM modulators include N-butylmannosamine or inhibitory nucleic acid molecules (e.g., small interfering RNA (siRNA), an anti-sense oligonucleotide (ASO), a short hairpin RNA (shRNA), a micro RNA (miRNA), or a double stranded RNA (dsRNA)) that target ST8 alpha-N-acetyl-neuraminide alpha-2,8-sialyltransferase 1 (ST8Sial) and/or ST8 alpha-N-acetyl-neuraminide alpha-2,8-sialyltransferase 5 (ST8SiaV) in the subject, thereby reducing ST8Sial and ST8SiaV mRNA and protein expression. An siRNA, ASO, shRNA, miRNA, or dsRNA can be designed to target an ST8Sial and/or ST8SiaV transcript sequence described in Table 3 below.

TABLE 3
Exemplary ST8Sial and ST8SiaV Transcript Sequences

| Name | NCBI Accession Numbers | Sequence Type |
|---|---|---|
| ST8 alpha-N-acetyl-neuraminide | NM_001304450.2 | Transcript Sequence |
| alpha-2,8-sialyltransferase 1 (ST8Sial) | NM_003034.4 | |
| ST8 alpha-N-acetyl-neuraminide | NM_001307987.2 | |
| alpha-2,8-sialyltransferase 5 | NM_013305.6 | |
| (ST8SiaV) | NM_001307986.2 | |

Prior to the administration of a composition containing a plurality of stem cells (e.g., MSCs, UC-MSCs, or NSCs), the subject may be treated (e.g., pre-treated) with one or more stem cell activators described herein (e.g., HDAC1 inhibitors, GSK-3B13 inhibitors, NK-1 receptor agonist, FGF proteins, nutraceuticals, Li, and NCAM modulators). Concurrently with the administration of a composition containing a plurality of stem cells (e.g., MSCs, UC-MSCs, or NSCs), the subject may be treated (e.g., co-treated) with one or more stem cell activators described herein (e.g., HDAC1 inhibitors, GSK-3B13 inhibitors, NK-1 receptor agonist, FGF proteins, nutraceuticals, Li, and NCAM modulators). Following the administration of a composition containing a plurality of stem cells (e.g., MSCs, UC-MSCs, or NSCs), the subject may be treated (e.g., post-treated) with one or more stem cell activators described herein (e.g., HDAC1 inhibitors, GSK-3Bβ inhibitors, NK-1 receptor agonist, FGF proteins, nutraceuticals, Li, and NCAM modulators). Thus, treatment with the one or more stem cell activators described herein is envisioned to occur prior to, concurrently with, and/or following the administration of a composition containing a plurality of stem cells (e.g., MSCs, UC-MSCs, or NSCs).

Prior to, concurrently with, and/or following the administration of a composition containing a plurality of stem cells (e.g., MSCs, UC-MSCs, or NSCs), the subject may be administered an amount of the HDAC1 inhibitor (e.g., one or more of romidepsin (FK-228), vorinostat, romidepsin, belinostat, panobinostat, VPA, entinostat, curcumin, quercetin, and RG2833) sufficient to achieve a serum concentration about 0.1 nM to about 1000 nM of the HDAC1 inhibitor (e.g., about 0.1 nM to 10 nM, about 1 nM to about 100 nM, about 10 nM to about 1000 nM, about 200 nM to about 500 nM, about 350 nM to about 700 nM, or about 500 nM to about 1000 nM, e.g., 01 nM, 0.5 nM, 1 nM, 5 nM, 10 nM, 20 nM, 30 nM, 40 nM, 50 nM, 60 nM, 70 nM, 80 nM, 90 nM, 100 nM, 125 nM, 150 nM, 175 nM, 200 nM, 225 nM, 250 nM, 275 nM, 300 nM, 325 nM, 350 nM, 375 nM, 400 nM, 425 nM, 450 nM, 475 nM, 500 nM, 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, or 1000 nM of the HDAC1 inhibitor). For compositions containing more than one HDAC1 inhibitor, these concentrations are envisioned for each HDAC1 inhibitor. In particular, the subject is administered an amount of an HDAC1 inhibitor (e.g., curcumin) sufficient to achieve a serum concentration of about 500 nM (e.g., 450 nM, 460 nM, 470 nM, 480 nM, 490 nM, 500 nM, 510 nM, 520 nM, 530 nM, 540 nM, or 550 nM). For example, the subject is administered an amount of an HDAC1 inhibitor (e.g., curcumin) sufficient to achieve a serum concentration of 500 nM.

Curcumin can act as a pleiotropic agent since it appears to act as both a HDAC1 inhibitor and a GSK-3 β inhibitor; for example, curcumin can alter the expression level of targets of both HDAC1 inhibitors and GSK-3β inhibitors (e.g., Sirt-1, CXCR4, HSP70, Oct 3/4, and FGF-21). In some embodiments, expression of Sirt-1 is increased by 200-to-300-fold, expression of CXCR4 is increased by 10-to-20-fold, HSP 70 levels are increased by up to 20-fold, Oct 3/4 is increased by approximately 10-to-20-fold, within the patient receiving the UC-MSCs. In other embodiments, expression of FGF-21 can be increased by 10-to-20-fold, and activation of CXCR4 and MMP9 can be increased by administering substances that induce GSK-3β inhibition and HDAC1 inhibition.

The HDAC1 inhibitor vorinostat (e.g., ZOLINZA®) may also be administered in an amount of about 400 mg (e.g., 360 mg, 370 mg, 380 mg, 390 mg, 400 mg, 410 mg, 420 mg, 430 mg, or 440 mg) and/or in an amount sufficient to achieve geometric mean values of a maximum plasma concentration (Cₘₐₓ) and an area under the plasma concentration versus time curve (AUC_{0-inf}) of 1.2±0.53 μM and 6.0±2.0 μM*hr, respectively. Vorinostat may be administered orally as a capsule or tablet.

The HADC1 inhibitor romidepsin (e.g., ISTODAX®) may also be administered in an amount of about 14 mg/m² (e.g., 12.6 mg/m², 13 mg/m², 13.5 mg/m², 14 mg/m², 14.5 mg/m², 15 mg/m², or 15.4 mg/m²) over a 4-hour period, such as on days 1, 8, and 15 of a 28-day cycle and/or in an amount sufficient to achieve geometric mean values of a maximum plasma concentration (Cₘₐₓ) and an area under the plasma concentration versus time curve (AUC_{0-inf}) of about 377 ng/mL (e.g., 340 ng/mL, 360 ng/mL, 380 ng/mL, 400 ng/mL, or 414 ng/mL) and about 1549 ng*hr/mL (e.g., 1395 ng*hr/mL, 1440 ng*hr/mL, 1480 ng*hr/mL, 1520 ng*hr/mL, 1560 ng*hr/mL, 1600 ng*hr/mL, 1640 ng*hr/mL, 1680 ng*hr/mL, or 1703 ng*hr/mL) respectively. Romidepsin may be administered by IV.

The HDAC1 inhibitor belinostat (e.g., BELEODAQ®) may also be administered in an amount of about 1,000 mg/m² (e.g., 900 mg/m², 950 mg/m², 1,000 mg/m², 1,050 mg/m², 1,100 mg/m²) over a 30 minute period, such as on days 1-5 of a 21-day cycle. Belinostat may be administered by IV (e.g., intravenous infusion).

The HDAC1 inhibitor panobinostat (FARYDAK®) may also be administered in an amount of about 20 mg (e.g., 18 mg, 19 mg, 20 mg, 21 mg, or 22 mg) every other day, such as on days 1, 3, 5, 8, 10, and 12 of a 21-day cycle. Panobinostat may be administered orally as a capsule or tablet.

The HDAC1 inhibitor valproic acid (e.g., DEPAKENE®) may also be administered in an amount of about 10 to 60 mg/kg/day (e.g., 10 to 40 mg/kg/day, 30 to 50 mg/kg/day or 40 to 60 mg/kg/day, e.g., 10 mg/kg/day, 15 mg/kg/day, 20 mg/kg/day, 25 mg/kg/day, 30 mg/kg/day, 35 mg/kg/day, 40 mg/kg/day, 45 mg/kg/day, 50 mg/kg/day, 55 mg/kg/day, or 60 mg/kg/day). Valproic acid may be administered orally as a capsule or tablet.

The HDAC1 inhibitor entinostat may also be administered in an amount of about 2 mg/m² to about 12 mg/m² (e.g., 2 mg/m² to 9 mg/m² or 6 mg/m² to 12 mg/m², e.g., 1 mg/m², 2 mg/m², 3 mg/m², 4 mg/m², 5 mg/m², 6 mg/m², 7 mg/m², 8 mg/m², 9 mg/m², or 10 mg/m², 11 mg/m², or 12 mg/m²). Entinostat may be administered orally as a capsule or tablet.

The HDAC1 inhibitor curcumin may also be administered in an amount of about 1 g to about 8 g per day (e.g., 1 g to 4 g per day, 2 g to 6 g per day, or 4 g to 8 g per day, e.g., 1 g per day, 2 g per day, 3 g per day, 4 g per day, 5 g per day, 6 g per day, 7 g per day, or 8 g per day).

The HDAC1 inhibitor quercetin may also be administered in an amount of about 250 mg to about 5000 mg per day (e.g., 250 mg to 1000 mg per day, 500 mg to 2000 mg per day, 1500 mg to 3000 mg per day, 2000 mg to 4000 mg per day, or 3000 mg to 5000 mg per day, e.g., 250 mg per day, 300 mg per day, 400 mg per day, 500 mg per day, 600 mg per day, 700 mg per day, 800 mg per day, 900 mg per day, 1000 mg per day, 1100 mg per day, 1200 mg per day, 1300 mg per day, 1400 mg per day, 1500 mg per day, 1600 mg per day, 1700 mg per day, 1800 mg per day, 1900 mg per day, 2000 mg per day, 2100 mg per day, 2200 mg per day, 2300 mg per day, 2400 mg per day, 2500 mg per day, 2600 mg per day, 2700 mg per day, 2800 mg per day, 2900 mg per day, 3000 mg per day, 3100 mg per day, 3200 mg per day, 3300 mg per day, 3400 mg per day, 3500 mg per day, 3600 mg per day, 3700 mg per day, 3800 mg per day, 3900 mg per day, 4000 mg per day, 4100 mg per day, 4200 mg per day, 4300 mg per day, 4400 mg per day, 4500 mg per day, 4600 mg per day, 4700 mg per day, 4800 mg per day, 4900 mg per day, or 5000 mg per day),

The HADC1 inhibitor RG2833 may also be administered in an amount of about 30 mg to about 240 mg per day (e.g., 30 mg to 100 mg per day, 60 mg to 150 mg per day, 100 mg to 200 mg per day, or 150 mg to 240 mg per day, e.g, 30 mg per day, 40 mg per day, 50 mg per day, 60 mg per day, 70 mg per day, 80 mg per day, 90 mg per day, 100 mg per day, 110 mg per day, 120 mg per day, 130 mg per day, 140 mg per day, 150 mg per day, 160 mg per day, 170 mg per day, 180 mg per day, 190 mg per day, 200 mg per day, 210 mg per day, 220 mg per day, 230 mg per day, or 240 mg per day).

Prior to, concurrently with, and/or following the administration of a composition containing a plurality of stem cells (e.g., MSCs, UC-MSCs, or NSCs), the subject may be administered an amount of the GSK-3β inhibitor (e.g., indirubin-3′-oxime) sufficient to achieve a serum concentration about 20 nM to about 500 nM (e.g., 16 nM, 20 nM, 30 nM, 40 nM, 50 nM, 60 nM, 70 nM, 80 nM, 90 nM, 100 nM, 125 nM, 150 nM, 175 nM, 200 nM, 225 nM, 250 nM, 275 nM, 300 nM, 325 nM, 350 nM, 375 nM, 400 nM, 425 nM, 450 nM, 475 nM, 500 nM, 525 nM, or 550 nM). In particular, the subject is administered an amount of an GSK-3β inhibitor (e.g., indirubin-3′-oxime) sufficient to achieve a serum concentration of about 200 nM (e.g., 160 nM, 170 nM, 180 nM, 190 nM, 200 nM, 210 nM, or 220 nM). For example, the subject is administered an amount of a GSK-3β inhibitor (e.g., indirubin-3′-oxime) sufficient to achieve a serum concentration of 500 nM.

Prior to, concurrently with, and/or following the administration of a composition containing a plurality of stem cells (e.g., MSCs, UC-MSCs, or NSCs), the subject may be administered an amount of a NK-1 receptor agonist (e.g., substance P) sufficient to achieve a serum concentration of about 1 nM to about 5 nM (e.g., about 1 nM to about 4 nM, about 1 nM to about 3 nM, or about 2 nM to about 3 nM, e.g., 1 nM, 1.5 nM, 2 nM, 2.1 nM, 2.2 nM, 2.3 nM, 2.4 nM, 2.5 nM, 2.6 nM, 2.7 nM, 2.8 nM, 2.9 nM, 3 nM, 3.5 nM, 4 nM, 4.5 nM, or 5 nM). For example, the subject may be administered an amount of an NK-1 receptor agonist (e.g., substance P) sufficient to achieve a serum concentration of about 2.5 nM.

Prior to, concurrently with, and/or following the administration of a composition containing a plurality of stem cells (e.g., MSCs, UC-MSCs, or NSCs), the subject may be administered an amount of an FGF protein (e.g., FGF-2) sufficient to achieve a serum concentration of about 3 ng/mL to about 10 ng/mL (e.g., about 3 ng/mL to about 6 ng/mL or about 3.5 ng/mL to about 4.5 ng/mL, e.g., 3 ng/mL, 3.5 ng/mL, 3.6 ng/mL, 3.7 ng/mL, 3.8 ng/mL, 3.9 ng/mL, 4 ng/mL, 4.1 ng/mL, 4.2 ng/mL, 4.3 ng/mL, 4.4 ng/mL, 4.5 ng/mL, 5 ng/mL, 6 ng/mL, 7 ng/mL, 8 ng/mL, 9 ng/mL, or 10 ng/mL). For example, the subject may be administered an amount of FGF-2 sufficient to achieve a serum concentration of about 4.2 ng/mL.

Prior to, concurrently with, and/or following the administration of a composition containing a plurality of stem cells (e.g., MSCs, UC-MSCs, or NSCs), the subject may be administered an amount of a nutraceutical (e.g., resveratrol, curcumin, or quercetin) sufficient to achieve a serum concentration of about 1 nM to about 10 μM (e.g., 1 nM, 50 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or 10 μM) about 100 nM to about 1 μM (e.g., 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, or 1 μM), about 400 nM to about 600 nM (e.g., 400 nM, 425 nM, 450 nM, 475 nM, 500 nM, 525 nM, 550 nM, 575 nM, or 600 nM), or about 500 nM (e.g., 450 nM, 460 nM, 470 nM, 480 nM, 490 nM, 500 nM, 510 nM, 520 nM, 530 nM, 540 nM, or 550 nM). For example, the subject may be administered an amount of a nutraceutical (e.g., resveratrol) sufficient to achieve a serum concentration of about 253 nM.

Prior to, concurrently with, and/or following the administration of a composition containing a plurality of stem cells (e.g., MSCs, UC-MSCs, or NSCs), the subject may be administered an amount of Li (e.g., lithium chloride) sufficient to achieve a serum concentration of about 10 μM to about 200 μM (e.g., 10 μM, 25 μM, 50 μM, 75 μM, 100 μM, 125 μM, 150 μM, 175 μM, or 200 PM) about 50 μM to about 125 μM (e.g., 50 μM, 70 μM, 90 μM, 110 μM, or 125 μM), about 75 μM to about 85 μM (e.g., 75 μM, 76 μM, 77 μM, 78 μM, 79 μM, 80 μM, 81 μM, 82 μM, 83 μM, 84 μM, or 85 μM), or about 79 μM (e.g., 71.1 μM, 72 μM, 73 μM, 74 μM, 75 μM, 76 μM, 77 μM, 78 μM, 78.5 μM, 79 μM, 79.5 μM, 80 μM, 81 μM, 82 μM, 83 μM, 84 μM, 85 μM, 86 μM, or 86.9 μM). For example, the subject may be administered an amount of Li (e.g., lithium chloride) sufficient to achieve a serum concentration of about 79 μM.

Prior to, concurrently with, and/or following the administration of a composition containing a plurality of stem cells (e.g., MSCs, UC-MSCs, or NSCs), the subject may be administered an amount of N-butylmannosamine sufficient to achieve a serum concentration of about 1 mM to about 50 mM (e.g., about 1 mM to about 25 mM, about 5 mM to about 30 mM, about 10 mM, to about 35 mM, about 15 mM, to about 40 mM, about 20 mM to about 45 mM, or about 25 mM to about 50 mM, e.g., about 1 mM, about 2 mM, about 3 mM, about 4 mM, about 5 mM, about 6 mM, about 7 mM, about 8 mM, about 9 mM, about 10 mM about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, or about 50 mM).

Prior to, concurrently with, and/or following the administration of a composition containing a plurality of stem cells (e.g., MSCs, UC-MSCs, or NSCs), the subject may be administered an amount of an siRNA, ASO, shRNA, miRNA, or dsRNA that target ST8Sial and/or ST8SiaV and reduces expression (e.g., mRNA and/or protein expression levels) of ST8Sial and/or ST8SiaV in a cell of the subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

Prior to, concurrently with, and/or following the administration of a composition containing a plurality of stem cells (e.g., MSCs, UC-MSCs, or NSCs), the subject may be administered one, two, three, four, five, or more of the stem cell activators described above (e.g., romidepsin (FK-228), vorinostat, romidepsin, belinostat, panobinostat, VPA, entinostat, curcumin, quercetin, RG2833, indirubin-3′-oxime, laduviglusib (CHIR-99821), KY19382, substance P, FGF-2, resveratrol, curcumin, N-butylmannosamine, or an inhibitory nucleic acid molecule targeting ST8Sial and/or ST8SiaV).

Any of the stem cell activators described herein (e.g., HDAC1 inhibitors, GSK-3B3 inhibitors, NK-1 receptor agonist, FGF proteins, nutraceuticals, Li, and NCAM modulators) may be administered to a subject with PASC in a volume of about 0.05 mL to about 15 mL (e.g., 0.05 mL, 0.1 mL, 0.2 mL, 0.3 mL, 0.4 mL, 0.5 mL, 0.6 mL, 0.7 mL, 0.8 mL, 0.9 mL, 1 mL, 3 mL, 5 mL, 7 mL, 10 mL, 11 mL, 12 mL, 13 mL, 14 mL, or 15 mL), about 1 mL to about 10 mL (e.g., 1 mL, 2 mL, 3 mL, 4 mL, 5 mL, 6 mL, 7 mL, 8 mL, 9 mL, or 10 mL), or about 1 mL to about 6 mL, (e.g., 1 mL, 1.25 mL, 1.5 mL, 1.75 mL, 2 mL, 2.25 mL, 2.5 mL, 2.75 mL, 3 mL, 3.25 mL, 3.5 mL, 3.75 mL, 4 mL, 4.25 mL, 4.5 mL, 4.75 mL, 5 mL, 5.25 mL, 5.5 mL, 5.75 mL, or 6 mL).

Any of stem cell activators described herein (e.g., HDAC1 inhibitors, GSK-3B3 inhibitors, NK-1 receptor agonist, FGF proteins, nutraceuticals, Li, and NCAM modulators) may be administered to a subject with PASC via a bolus (e.g., an IV bolus), a push (e.g., an IV push), or a drip (e.g., an IV drip). Further, administration of the stem cell activator may be over a period of 1 minute to 1 hour (e.g., 1 minute, 10 minutes, 20 minutes, 30 minutes, 40 minutes, 50 minutes, or 1 hour), 1 minute to 30 minutes (e.g., 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, or 30 minutes), or about 1 minute to about 10 minutes (e.g., 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, or 10 minutes).

Administration of the stem cell activator may occur prior to, concurrently with, and/or following the administration of a composition described herein. Serum concentrations of the stem cell activators may be assessed by standard laboratory techniques, such as enzymed-linked immunosorbent assay (ELISA), Chemiluminescent immunoassays (CLIAs), or using a Beckman Coulter Clinical Chemistry Analyzer.

Analysis of stem cell activation according to the present disclosure also includes neuroimaging methods to assess NSC status within treated patients. While many imaging procedures provide information on multi-cellular structures, other methods well-known in the art allow imaging at a cellular level. Pyrimidines are selectively taken up by proliferating cells and preferred, without limiting, method of NSC proliferation imaging of the present disclosure is by positron-emission tomography of F-labeled 3′-deoxy-3′-fluor thymidine at known anatomical NSC niches including the SVZ of the lateral ventricles. Other embodiments of this method of direct imaging of stem cell proliferation and gene expression profiling are readily apparent to those skilled in the art.

### C. PASC Subjects in Need of Treatment

Further embodiments include use of various clinical endpoints to assess the safety and/or efficacy of the above treatments in a subject with persistent symptoms and/or delayed or long-term complications of SARS-CoV-2 (COVID-19) (PASC) or the SARS-CoV-2 vaccine beyond four weeks from the onset of symptoms or vaccine administration, as assessed by the incidence of adverse events (AEs) and serious adverse events (SAEs). An AE is any noxious, unintended, or untoward medical occurrence that may appear or worsen in a subject during a study, whether considered to be related to study treatment or not and can be an abnormal laboratory value. An SAE is any AE that suggests a significant hazard or AE, whether considered to be related to study treatment. An SAE fulfills one or more of the following criteria:
Results in death
Is life-threatening (i.e., in the opinion of the Investigator, the subject is at immediate risk of death from the AE as it occurred).
Requires in-subject hospitalization or prolongation of existing hospitalization (hospitalization is defined as an in-subject admission, regardless of length of stay).
Results in persistent or significant disability or incapacity (a substantial disruption of the subject's ability to conduct normal life functions).
Results in a congenital abnormality or birth defect.
Is an important medical event that may jeopardize the subject or may require medical intervention to prevent one of the outcomes listed above. Important medical events are defined as those occurrences that may not be immediately life-threatening or result in death, hospitalization, or disability, but may jeopardize the subject or require medical or surgical intervention to prevent one of the other outcomes listed above. Medical and scientific judgment should be exercised in deciding whether such an AE should be considered serious.

Efficacy evaluations include assessment of fatigue by FACIT-F patient reported outcome, post-exertion problems, cognitive impairment, joint or muscle pain, numbness, diarrhea, sleep impairment, dizziness when standing, skin rash, mood changes, and loss of smell and taste. Efficacy assessment also includes return to baseline clinical laboratory measures of lymphocytes, hemoglobin, platelets, C3 & C4, CRP, D-dimer, IgM, IgG, IgA, B cells, T cells, CD4/CD8 T cell ratio & NK cells. Additional endpoints include anti-nuclear antibody (ANA) titer and autoantibody quantitation to the autoantigens: GO:0043043, R-HAS-8953854, CORUM:1181, GO:0045055 including other identified COVID-19 induced autoantigens totaling 191 proteins (Wang, J Y, et al, J. Autoimmunity 120: 102644, 2021) (e.g., see Table 4 below). Neurological efficacy assessments include cognitive function testing, brain imaging by SPECT and MRI.

TABLE 4
Autoantigens

| Gene | Protein Name | Gene | Protein Name |
|---|---|---|---|
| EEF1A2 | Elongation factor 1-alpha 2 | HIST1H1C | Histone H1.2 |
| EEF1G | Elongation factor 1-gamma | HIST1H2BL | Histone H2B type 1-L, H2BC13 |
| EEF2 | Elongation factor 2 | HIST1H4J | Histone H4, H4C1 |
| EIF3A | Eukaryotic translation initiation | HIST2H2BE | Histone H2B type 2-E, H2BC21 |
| | factor 3 subunit A | | |
| EIF3B | Eukaryotic translation initiation | HIST2H3A | HIST2H3C, Histone H3.2, |
| | factor 3 subunit | | H3C15 |
| EIF3E | Eukaryotic translation initiation | HNRNPA2B1 | Heterogenous nuclear |
| | factor 3 subunit E | | ribonucleoproteins A2/B1 |
| EIF4A1 | Eukaryotic initiation factor 4A-I, | HNRNPC | Heterogeneous nuclear |
| | DDX2A | | ribonucleoproteins C1/C2 |
| EIF5A | Eukaryotic translation initiation | HNRNPH1 | Heterogeneous nuclear |
| | factor 5A-1 | | ribonucleoprotein H |
| EMG1 | Ribosomal RNA small subunit | HNRNPK | Heterogeneous nuclear |
| | methyltransferase NEP1 | | ribonucleoprotein K |
| ENO1 | Isoform alpha-enolase of | HNRNPM | Heterogeneous nuclear |
| | Alpha-enolase | | ribonucleoprotein M |
| EZR | Ezrin | HNRNPR | Heterogeneous nuclear |
| | | | ribonucleoprotein R |
| FLNA | Filamin-A | HNRNPU | Heterogeneous nuclear |
| | | | ribonucleoprotein U |
| FLNB | Filamin-B | HNRNPUL1 | hnRNP_U-like protein 1 |
| FLNC | Filamin-C | HNRNPUL2 | hnRNP_U-like protein 2 |
| FTH1 | Ferritin heavy chain | HSP90AA1 | Heat shock 90 kDa protein 1, |
| | | | alpha isoform |
| G6PD | Glucose-6-phosphate 1- | HSP90AA2 | Heat shock protein HSP 90- |
| | dehydrogenase (anemia, | | alpha A2 |
| | diabetes) | | |
| GAPDH | Glyceraldehyde-3-phosphate | HSP90AB1 | Heat shock protein HSP 90- |
| | dehydrogenase | | beta |
| GBE1 | 1,4-alpha-glucan-branching | HSP90B1 | Endoplasmin |
| | enzyme | | |
| GDI1 | Rab GDP dissociation inhibitor | HSPA1A | Heat shock 70 kDa protein 1A |
| | alpha | | |
| GDI2 | CDNA FLJ60299, highly similar | HSPA5 | HSPA5 protein, BiP, GRP78 |
| | to Rab GDP dissociation | | |
| | inhibitor beta | | |
| GPI | Glucose-6-phosphate | HSP70B | Putative heat shock 70 kDa |
| | isomerase | | protein, HSPA7 |
| GSTP1 | Glutathione S-transferase | HSPA8 | Heat shock cognate 71 kDa |
| | | | protein |
| H2AFY | H2A histone family, member Y | HSPA9 | Stress-70 protein, mitochondrial |
| | isoform, MacroH2A1 | | |
| HEATR1 | HEAT repeat-containing protein | HSPBP1 | Hsp70-binding protein |
| | 1 | | |
| HIST1H1B | Histone H1.5, H1-5 | HSPG2 | Heparan sulfate proteoglycan 2 |

The methods of treatment described herein are intended to treat a subject with PASO. A subject with PASO may exhibit one or more symptoms selected from: cognitive impairment, joint pain, muscle pain, chest pain, stomach pain, headache, tachycardia, numbness, diarrhea, brain fog, fatigue, sleep impairment, dizziness when standing, shortness of breath, fever, skin rash, mood changes, loss of smell, exercise intolerance, and/or loss of taste. The symptoms may result from a previous SARS-CoV-2 infection, an ongoing SARS-CoV-2 infection, or a SARS-CoV-2 vaccine. The subject may be treated for PASC following the methods described herein after their diagnosis of PASC (e.g., within 1 hour, within 1 day, within 1 week, within 1 month, within 6 months, or within 1 year from the subject's diagnosis) or after at least 4 weeks (e.g., 4 weeks, 5 weeks, 6 weeks, 7 weeks, 8 weeks, 9 weeks, 10 weeks, or more) of experiencing at least one of the aforementioned symptoms.

Any subject with PASC may be administered a composition described herein (e.g., a composition containing a plurality of stem cells (e.g., MSCs, UC-MSCs, or NSCs), a plurality of isolated exosomes, stem cell (e.g., MSC, UC-MSC, or NSC) secretome-conditioned cell culture medium, exosome-depleted stem cell (e.g., MSC, UC-MSC, or NSC) secretome-conditioned cell culture medium, resveratrol, curcumin, quercetin, or some combination thereof) using the methods described herein. For example, the subject may be administered the composition by IV infusion at 1×10⁶ to 2.5×10⁶ cells/kg of body weight or a total of 5×10⁷ to 1×10⁸ (e.g., 1×10⁸) stem cells (e.g., MSCs, UC-MSCs, or NSCs). The composition may further include an HDAC1 inhibitor (e.g., 500 nM of curcumin), which can stimulate stem cell activation. Further, administration may occur 1 to 4 times, 2 to 4 times, or 3 to 4 times per year. The number of administrations per year can depend on need, as determined can a clinician. In another example, the subject may be administered a composition containing a nutraceutical, such as resveratrol (e.g., about 253 nM of resveratrol), curcumin (e.g., about 500 nM of curcumin), and/or quercetin (e.g., about 500 nM of quercetin). Further, administration may occur 1 to 4 times, 2 to 4 times, 3 to 4 times, 4 to 5 times, 5 to 6 times, or 6 to 7 times per month. The number of administrations per month can depend on need, as determined can a clinician.

In severe cases of PASC, the subject may be suffering from an organ failure (e.g., renal, hepatic, respiratory, or nervous system failure). In such cases, the subject may also receive ventilatory support.

A subject with PASC may exhibit an abnormal clinical laboratory measurement such as: a lymphocyte count outside the range of 4-12×10⁹/L; a hemoglobin level less than 12.3 g/dL; a platelet level outside the range of 150-440×10⁹/L; a C3 level outside the range of 80-178 mg/dL; a C4 level greater than or equal to 12-42 mg/dL; a CRP level greater than 1 mg/dL; a d-dimer level greater than 500 ng/mL; an IgM level greater than 240 mg/dL; an IgG level greater than 1600 mg/dL; an IgA level greater than 450 mg/dL; a B cell count greater than or equal to 100-600×10⁶/L; a T cell count greater than or equal to 0.64-1.18×10⁹/L; a CD4/CD8 T cell ratio greater than 1.0; a natural killer (NK) cell count greater than 100×10⁶/L; an anti-nuclear antibody (ANA) titer greater than 1:160; and any positive autoantibody quantitation to autoantigens listed in Table 4; a blood urea nitrogen (BUN) level greater than 25 mEq/L; a creatine level greater than 1.5 mEq/L; an aspartate transaminase (AST) level greater than 33 U/L; an alanine aminotransferase (ALT) level greater than 36 U/L; an alkaline phosphatase level greater than 115 IU/L; a white blood cell (WBC) count greater than 12×10⁹¹; a lymphocyte count of about 800 cells per μL; a lactic acid level greater than 4.0 mmol/L; a procalcitonin level greater than 0.25 ng/mL; a lactate dehydrogenase (LDH) level greater than 240 U/L; a ferritin level greater than 284 ng/mL; a c-reactive protein (CRP) level greater than 1.0 mg/dL; and/or a d-dimer level greater than 500 ng/mL.

Treatment of a subject with PASC as described herein may resolve any abnormal clinical laboratory measurement in the subject, such that one or more of the measurements are restored to a baseline (or normal range) clinical laboratory measurement. For example, upon administration of a composition described herein (e.g., a composition containing a plurality of stem cells (e.g., MSCs, UC-MSCs, or NSCs), a plurality of isolated exosomes, stem cell (e.g., MSC, UC-MSC, or NSC) secretome-conditioned cell culture medium, exosome-depleted stem cell (e.g., MSC, UC-MSC, or NSC) secretome-conditioned cell culture medium, resveratrol, curcumin, quercetin, or some combination thereof), the subject may exhibit a lymphocyte count of about 4-12×10⁹/L; a hemoglobin level of about 12.3-15.7 g/dL; a platelet level of about 150-440×10⁹/L; a C3 level of about 80-178 mg/dL; a C4 level of about 12-42 mg/dL; a CRP level of about 0.3-1 mg/dL; a d-dimer level of less than 500 ng/mL; an IgM level of about 40-240 mg/dL; an IgG level of about 600-1600 mg/dL; an IgA level of about 80-450 mg/dL; a B cell count of about 100-600×10⁶/L; a T cell count of about 0.64-1.18×10⁹/L; a CD4/CD8 T cell ratio of less than 1.0; a NK cell count greater than 100×10⁶/L; an ANA titer less than 1:160; no detectable level of an autoantigen listed in Table 4; a BUN level of about 8-25 mEq/L; a creatine level of about 0.5-1.5 mEq/L; an AST level of about 8-33 U/L; an ALT level of about 4-36 U/L; an alkaline phosphatase level of about 25-115 U/L; a WBC count of about 4-12×10⁹/L with about 40-60% neutrophilic predominance and lymphocyte component of about 20-40%; a lactic acid level of about 2-4 mM; a procalcitonin level of about 0.1-0.25 ng/mL; an LDH level of about 50-240 U/L; a ferritin level of about 30-284 ng/mL; a CRP level of about 0.3-1.0 mg/dL; and/or a d-dimer level of less than 500 ng/mL.

A subject with PASC may exhibit an abnormal measurement during an arterial blood gas (ABG) test, such as: an arterial blood pH level outside the range of 7.37-7.44, such as about 7.1; a partial pressure of carbon dioxide (pCO₂) outside the range of 34-43 mm Hg, such as about 60 mm Hg; a partial pressure of oxygen (pO₂) and fraction of inspired oxygen (FiO₂) ratio (pO₂/FiO₂) of less than 300, such as about 255; and/or a positive end-expiratory pressure (PEEP) of less than 30 cm H₂O.

Treatment of a subject with PASC as described herein may resolve any abnormal measurement in the ABG test, such that one or more of the measurements are restored to a baseline (or normal range) clinical laboratory measurement. For example, upon administration of a composition described herein (e.g., a composition containing a plurality of stem cells (e.g., MSCs, UC-MSCs, or NSCs), a plurality of isolated exosomes, stem cell (e.g., MSC, UC-MSC, or NSC) secretome-conditioned cell culture medium, exosome-depleted stem cell (e.g., MSC, UC-MSC, or NSC) secretome-conditioned cell culture medium, resveratrol, curcumin, quercetin, or some combination thereof), the subject may exhibit a pH level within the range of 7.35-7.45, such as about 7.435; a pCO₂ within the range of 37-45 mm Hg, such as about 43.3 mm Hg; a pO₂/FiO₂ greater than 300, such as about 316; and/or a PEEP of less than 30 cm H₂O

### II. Compositions

The composition described herein contain human stem cells (e.g., MSCs, UC-MSCs, and NSCs) isolated exosomes derived from the stem cells, cell culture medium containing the stem cell's secretome (e.g., stem cell secretome-conditioned cell culture medium), cell culture medium containing the stem cell's secretome but devoid of exosomes (e.g., exosome-depleted stem cell secretome-conditioned cell culture medium), a nutraceutical (e.g., resveratrol, curcumin, or quercetin) or some combination thereof. Compositions may further include one or more stem cell activators, such as any amount of an HDAC1 inhibitor (e.g., romidepsin (FK-228), vorinostat, romidepsin, belinostat, panobinostat, VPA, entinostat, curcumin, quercetin, and RG2833), a GSK-3B3 inhibitor (e.g., indirubin-3′-oxime, laduviglusib (CHIR-99821), and KY19382), a NK-1 receptor agonist (e.g., substance P), a FGF protein (e.g., FGF-2), a nutraceutical (e.g., resveratrol, curcumin, and quercetin), Li (e.g., lithium chloride), and an NCAM modulator (e.g., N-butylmannosamine or inhibitory nucleic acid molecules that target ST8Sial and/or ST8SiaV) described herein.

### A. Stem Cells

Stem cells of the present disclosure can be obtained or derived from a variety of tissues including adipose tissue, umbilical cord, placenta, amniotic fluid or membranes, umbilical cord blood, bone marrow, and other sources well known to those skilled in the art. The MSCs may be extracted from tissue through a combination of microdissection followed by enzymatic or mechanical dissociation. Donor screening can be used to eliminate those with pre-existing conditions that may impose a safety risk for use of stem cells in clinical applications. MSCs can be purified by a variety of methods, such as flow cytometry or selective adsorption to plastic. Expansion can occur by standard methods of cell culture, preferably performed under conditions of reduced oxygen (1% to 5%) that represent oxygen levels within the native niches of MSCs within the body such as bone marrow. Expansion of cells can also occur by serial passage in cell culture using various methods of cellular dissociation from culture flasks/plates well known to those skilled in the art. Both 2D monolayer cultures in multi-layer culture flasks and 3D cultures in stirred tank bioreactors, hollow fiber cartridges and related configurations are embodied in the present disclosure.

MSCs can then be characterized by a variety of methods to ensure authenticity by function including differentiation capacity, growth rate, ATP production, expression of indoleamine 2,3-dioxygenbase induced by γ-IFN and other MSC functional characteristics known to those skilled in the art. Also, phenotypic characterization can be performed using flow cytometry to determine the absence or presence of specific cellular biomarkers. Karyotyping, DNA finger printing, and other well-known methods can be used to authenticate the species of origin and elucidate the genome of the MSC line. Adventitious viral agents can be determined by specific PCR methods or broader in-vitro and in-vivo methods detecting known and unknown viruses. Detection of bacteria, fungi, and viruses can be used to eliminate transmission of these agents during stem cell transplantation. Clinical use of MSCs can include rigorous adventitious agent testing by in-vitro cultures of well characterized cell lines, in-vivo testing in various animal species, and testing of any animal-derived products used in the manufacturing process.

The MSC lines derived by the above methods can be formulated by various methods including cryopreservation, viable liquid formulations that are suitable for administration into a patient through various routes including both systemic and local applications. In some aspects in accordance with the present disclosure, the MSCs can be genetically modified prior to clinical use, and in some embodiments secretion products of stem cells including exosomes or conditioned media derived from cultured MSCs may be used instead of MSCs or with MSCs.

The MSCs can be distributed to various administration sites in suitable, stable formulations to accommodate logistic requirements including temperature maintenance, and continuous monitoring of environmental parameters. Following recovery from cryopreservation by methods well-known to those skilled in the art as needed, stem cells can be administered to a patient in need by various routes of administration, including, but not limited to, intravenous infusion, intraarticular injection, intra-spinal-disc injection, intranasal, oral administration, or other methods of systemic administration or local injection or implantation well known in the art, including combination with stabilizing agents such as extracellular materials, either natural or synthetic. Several medical conditions are suitable for the stem cell therapies while the present disclosure embodies MSC therapy for PASC also referred to as Long COVID a relatively new disorder resulting from the COVID-19 pandemic.

The present disclosure provides compositions containing about 5×10⁵ to 5×10⁶ (e.g., 5×10⁵ to 1×10⁶, 5×10⁵ to 1.5×10⁶, 1×10⁶ to 2.5×10⁶, 1.5×10⁶ to 3×10⁶, 2×10⁶ to 5×10⁶, or 4×10⁶ to 5×10⁶, e.g., 5×10⁵, 6×10⁵, 7×10⁵, 8×10⁵, 9×10⁵, 1×10⁶, 1.5×10⁶, 2×10⁶, 2.5×10⁶, 3×10⁶, 3.5×10⁶, 4×10⁶, 4.5×10⁶, or 5×10⁶) of the isolated stem cells (e.g., MSCs, UC-MSCs, or NSCs) per kg of body weight. For example, a composition may contain 1×10⁶ to 2.5×10⁶ (e.g., 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, or 2.5×10⁶) of the isolated stem cells (e.g., MSCs, UC-MSCs, or NSCs) per body weight. In another example, a composition may contain about 1×10⁶ (e.g., 9×10⁵, 1×10⁶, or 1.1×10⁶) of the isolated stem cells (e.g., MSCs, UC-MSCs, or NSCs) per body weight.

In yet another example, a composition may contain a total of about 5×10⁷ to 1×10⁸ (e.g., 5×10⁷ to 6×10⁷, 6×10⁷ to 7×10⁷, 7×10⁷ to 8×10⁷, 8×10⁷ to 9×10⁷, or 9×10⁷ to 1×10⁸, e.g., 5×10⁷, 6×10⁷, 7×10⁷, 8×10⁷, 9×10⁷, or 1×10⁸) of the isolated stem cells (e.g., MSCs, UC-MSCs, or NSCs). In particular, a composition may contain total of about 1×10⁸ (e.g., 9×10⁷, 9.1×10⁷, 9.2×10⁷, 9.3×10⁷, 9.4×10⁷, 9.5×10⁷, 9.6×10⁷, 9.7×10⁷, 9.8×10⁷, 9.9×10⁷, 1×10⁸, or 1.1×10⁸), of the stem cells (e.g., MSCs, UC-MSCs, or NSCs).

Isolated stem cells (e.g., MSCs, UC-MSCs, or NSCs) may be isolated from the subject with PASC or isolated from a healthy donor (e.g., a subject that does not have PASC). Isolated stem cells (e.g., MSCs, UC-MSCs, or NSCs) may be allogenic or autologous.

Compositions containing isolated stem cells (e.g., MSCs, UC-MSCs, or NSCs) may further include one or more stem cell activators, such as any amount of an HDAC1 inhibitor (e.g., romidepsin (FK-228), vorinostat, romidepsin, belinostat, panobinostat, VPA, entinostat, curcumin, quercetin, and RG2833), a GSK-3Bβ inhibitor (e.g., indirubin-3′-oxime, laduviglusib (CHIR-99821), and KY19382), a NK-1 receptor agonist (e.g., substance P), a FGF protein (e.g., FGF-2), a nutraceutical (e.g., resveratrol, curcumin, and quercetin), Li (e.g., lithium chloride), and an NCAM modulator (e.g., N-butylmannosamine or inhibitory nucleic acid molecules that target ST8Sial and/or ST8SiaV) as described herein.

Compositions containing isolated stem cells (e.g., MSCs, UC-MSCs, or NSCs) may further include a pharmaceutically acceptable carrier, excipient, or diluent, which may contain an amount of piperine and may not contain DMSO. Compositions containing UC-MSCs may further include a cryopreservation medium, a basal medium, and/or a saline solution. The cryopreservation medium may be PRIME-XV® MSC FreeziS DMSO-Free medium (e.g., FUJIFILM Irvine Scientific Catalog number: 91140). The basal medium may be MCDB-131.

The stem cells of the composition may be modified as described herein.

i. Modified Stem Cells

Modified stem cells (e.g., MSCs, UC-MSCs, or NSCs) of the compositions described herein may be genetically modified to increase stem cell migration and proliferation phenotypes.

Stem cells (e.g., MSCs, UC-MSCs, or NSCs) may be isolated from the subject with PASC or isolated from a healthy donor (e.g., a subject that does not have PASC). Stem cells (e.g., MSCs, UC-MSCs, or NSCs) may be modified (e.g., modified stem cells, e.g., modified MSCs, modified UC-MSCs, or modified NSCs) as described herein. For example, stem cells (e.g., MSCs, UC-MSCs, or NSCs) may be genetically modified to express one, two, three, four, five, or more copies of FGF-2 and/or substance P (e.g., as mRNA and/or protein). Upon transfection, the modified stem cells may have an increased expression of Sirt-1, CXCR4, HSP70, Oct 3/4, and/or FGF-21 mRNA and/or protein levels relative to unmodified stem cells.

Stem cells (e.g., MSCs, UC-MSCs, or NSCs) may be modified by introducing exogenous genetic material (e.g., DNA, cDNA, RNA, or mRNA) into the stem cell. For example, a nucleic acid vector, plasmid, circular RNA, or mRNA molecule may be introduced into the stem cells, e.g., by way of transfection (e.g., microinjection, optical transfection, biolistic transfection, electroporation, nucleofection, lipofection, sonoporation, and magnetofection). The exogenous genetic material (e.g., nucleic acid vector, plasmid, circular RNA, or mRNA molecule) may contain one or more (e.g., 1, 2, 3, 4, 5, 6, 7, 8, 9, 10 or more) nucleotide sequences encoding one or more copies of the following genes: FGF-2 and/or substance P. This expression can augment the stem cells proliferation and migration phenotypes, thereby causing the stem cell to be activated.

Exemplary genes, mRNA transcripts, and protein sequences to be expressed in the stem cells are provided in Table 1.

ii. Quality Control Standards for UC-MSC Production

The stem cell compositions described herein may contain purified (>95% isolated), expanded, and cryogenically preserved human UC-MSCs in a non DMSO-containing excipient containing 50 million cells per vial at 10-12.5 million cells per mL. The donated umbilical cords can be obtained from American Association of Tissue Banks (AATB)-certified third-party providers. Since we use the human umbilical cord as a source of the MSCs, we have established strict criteria for the selection of full-term, donated umbilical cords for use in processing to purified ALLORX STEM CELLS®. First these tissues are only procured from AATB Accredited tissue suppliers. We manage tissue providers as per the ISO 9001:2015 quality standard and the ISO13485:2016 Medical Device Manufacturing Standard. All testing occurs using FDA approved assays.

The acceptance criteria include:
No history of sexually transmitted diseases (STD's) and tuberculosis (TB)
Freedom from risk factors for, or clinical evidence of, Zika Virus
No history of cancer or chronic illnesses
No history of autoimmunity disorders (e.g., human immunodeficiency virus (HIV), Type I Diabetes, MS, Lupus, etc.)
No tattoos within the past 12 months
No history of Creutzfeldt-Jakob disease (CJD) or prion disease
No history of drug or alcohol abuse

Tested negative by FDA-approved assays for adenovirus, Epstein-Barr (EBV), hepatitis A, hepatitis B core total antibody (Ab), hepatitis B surface antigen, hepatitis C virus Ab, human herpesvirus (HHV)-6, HHV-7, HHV-8, HIV-1/HIV-2, HTLV I/II Ab, Parvovirus 19, CMV HbSAg, HCV, HTLV I/I1, HIV I/I1, CMV, EBV, WNV, COVID-19 & Syphilis.

In addition to the selection criteria for donated umbilical cords, we perform a detailed lot specific final quality testing process before QC release of finished product including identity testing by the International Society for Cell and Gene Therapy (ISCT) phenotype standard for MSC identity: CD11b−; CD14−; CD19−; CD34−; CD44+; CD45−; CD73+; CD79a−; CD90+, CD105+; CD126−; HLA-DR-by flow cytometry. Demonstrated tri-lineage differentiation. Human karyotype and human DNA fingerprint test. Purity >95% by flow cytometry. Potency to QC release criteria by cellular ATP content and gamma-interferon induced IPO activity. Adventitious agent testing of quality includes negative 14-day USP-71 sterility testing, negative by PCR testing for 16S, 18S and mycoplasma; negative in-vitro cell-based viral testing in Hela cells, MCR-5 and Vero76 cells, negative in-vivo viral tests in guinea pigs, post-weaning mice, suckling mice and embryonic chick embryos via yolk sack and allantoic fluid deployment. Negative human viral pathogen tests by PCR for Parvovirus B19, Polyomavirus BKV, EBV, HAV, HBV, HCMV, HCV, HEV, HHV-6, HHV-7, HHV-8, HIV-1, HIV-2, HPV-16, HPV-18, HTLV-1, HTLV-2, JCV and SARS-Cov-2.

The procedures used in manufacturing and QC of UC-MSCs are described below in Examples 13 and 14.

### B. Isolated Exosomes

The present disclosure provides compositions containing a concentration of about 5×10⁹ to 5×10¹⁰ isolated exosomes per mL (e.g., 5×10⁹ to 1.5×10¹⁰, 1×10¹⁰ to 3×10¹⁰, 2×10¹⁰ to 4×10¹⁰, 3×10¹⁰ to 5×10¹⁰, 1×10¹⁰ to 5×10¹⁰, or 2.5×10¹⁰ to 5×10¹⁰, e.g., 5×10⁹, 6×10⁹, 7×10⁹, 8×10⁹, 9×10⁹, 1×10¹⁰, 1.5×10¹⁰, 2×10¹⁰, 2.5×10¹⁰, 3×10¹⁰, 3.5×10¹⁰, 4×10¹⁰, 4.5×10¹⁰, or 5×10¹⁰ isolated exosomes per mL). The present disclosure also provides compositions containing about 100 μg to about 300 μg of the isolated exosomes (e.g., 100 μg to 200 μg, 125 μg to 175 μg, or 200 μg to 300 μg, e.g., 100 μg, 125 μg, 150 μg, 175 μg, 200 μg, 225 μg, 250 μg, 275 μg, or 300 μg of the isolated exosomes).

The isolated exosomes may be derived from stem cell secretome-conditioned cell culture medium. After removing exosomes from stem cell secretome-conditioned cell culture medium, exosomes may contain a volume (e.g., a volume of about 2.7×10⁻¹⁰ mm³ or less) of the cell culture medium from which they were isolated from; this is still considered an “isolated” exosome.

The isolated exosomes of the present disclosure are lipid bilayer vesicles having a diameter of 80 nm to 200 nm (e.g., 80 nm, 90 nm, 100 nm, 110 nm, 120 nm, 130 nm, 140 nm, 150 nm, 160 nm, 170 nm, 180 nm, 190 nm, or 200 nm) and are isolated at purity greater than 90% (e.g., 90%, 91%, 92%, 93%, 94%, 95%, 96%, 97%, 98%, 99%, or 100%). These exosomes express at least CD9, CD63, CD81, CD44, CD29, and CD142. The isolated exosomes may not express CD45, CD11b, CD14, CD19, CD34−, CD79a, CD126, and human leukocyte antigen-DR isotype (HLD-DR).

Compositions containing isolated exosomes may further include one or more stem cell activators, such as any amount of an HDAC1 inhibitor (e.g., romidepsin (FK-228), vorinostat, romidepsin, belinostat, panobinostat, VPA, entinostat, curcumin, quercetin, and RG2833), a GSK-3B3 inhibitor (e.g., indirubin-3′-oxime, laduviglusib (CHIR-99821), and KY19382), a NK-1 receptor agonist (e.g., substance P), a FGF protein (e.g., FGF-2), a nutraceutical (e.g., resveratrol, curcumin, and quercetin), Li (e.g., lithium chloride), and/or an NCAM modulator (e.g., N-butylmannosamine or inhibitory nucleic acid molecules that target ST8Sial and/or ST8SiaV) as described herein.

### C. UC-MSC Secretome-Conditioned Cell Culture Medium

Compositions containing biological materials secreted from the UC-MSC (e.g., UC-MSC secretome-conditioned cell culture medium) are also envisioned for use in the treatment of PASC. Therapeutic benefits of stem cell therapy are thought to be in part mediated by soluble factors secreted from stem cells, collectively known as paracrine effects. The secretome is also referred to as UC-MSC-conditioned medium since the manufacturing process involves collection of the cell culture medium exposed to UC-MSCs maintained in cell cultures.

The MSC secretome consists of all secreted factors, including exosomes that are lipid bilayer vesicles of 140 to 200 nm diameter comprised of integral membrane proteins including the exosome-specific biomarkers CD9, CD61 and CD83 and various biological molecules including proteins, lipids, RNA, miRNA, DNA, fats contained within exosomes combined with other soluble factors secreted from MSCs and/or exosomes. Therapy may be mediated by the large variety of secreted factors derived from the stem cell secretome.

The UC-MSC secretome-conditioned cell culture medium may contain GM-CSF, MIP-3a, IL-6, and IL-8 each at about 500-2000 pg/mL (e.g., 500-1000 pg/mL, 800-1600 pg/mL, or 1400-2000 pg/mL, e.g., 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, 1000 pg/mL, 1050 pg/mL, 1100 pg/mL, 1150 pg/mL, 1200 pg/mL, 1250 pg/mL, 1300 pg/mL, 1350 pg/mL, 1400 pg/mL, 1450 pg/mL, 1500 pg/mL, 1550 pg/mL, 1600 pg/mL, 1650 pg/mL, 1700 pg/mL, 1750 pg/mL, 1800 pg/mL, 1850 pg/mL, 1900 pg/mL, 1950 pg/mL, 2000 pg/mL). The UC-MSC secretome-conditioned cell culture medium may further contain about 10 pg/mL to 500 pg/mL of fractalkine and MIP-1 (e.g., 10 pg/mL to 100 pg/mL, 10 pg/mL to 500 pg/mL, or 500 pg/mL to 1000 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, 250 pg/mL, 300 pg/mL, 350 pg/mL, 400 pg/mL, 450 pg/mL, or 500 pg/mL of fractalkine and MIP-1).

Compositions containing stem cell secretome-conditioned cell culture medium may further include one or more stem cell activators, such as any amount of an HDAC1 inhibitor (e.g., romidepsin (FK-228), vorinostat, romidepsin, belinostat, panobinostat, VPA, entinostat, curcumin, quercetin, and RG2833), a GSK-3B3 inhibitor (e.g., indirubin-3′-oxime, laduviglusib (CHIR-99821), and KY19382), a NK-1 receptor agonist (e.g., substance P), a FGF protein (e.g., FGF-2), a nutraceutical (e.g., resveratrol, curcumin, and quercetin), Li (e.g., lithium chloride), and/or an NCAM modulator (e.g., N-butylmannosamine or inhibitory nucleic acid molecules that target ST8Sial and/or ST8SiaV) described herein.

### D. Exosome-Depleted Stem Cell Secretome-Conditioned Cell Culture Medium

Exosome-depleted stem cell seretome-conditioned cell culture medium is derived from stem cell secretome-conditioned cell culture medium with a difference being that exosomes (e.g., exosomes that are of a specific size, e.g., 80-200 nm) have been removed. Several standard laboratory techniques exist to remove exosomes from cell culture medium, such as differential ultracentrifugation, seize exclusion chromatography, ultrafiltration, polyethylene glycol-based precipitation, immunoaffinity capture, or by using microfluidic devices. Exosome-depleted stem cell secretome-conditioned cell culture medium may contain other stem cell-derived biological material, such as proteins, lipids, and extracellular vesicles smaller than 80 nm or larger than 200 nm.

Exosome-depleted stem cell secretome-conditioned cell culture medium may also contain GM-CSF, MIP-3a, IL-6, and IL-8 each at about 100-2000 pg/mL (e.g., 500 pg/mL to 1000 pg/mL, 800 pg/mL to 1600 pg/mL, or 1400 pg/mL to 2000 pg/mL, e.g., 500 pg/mL, 550 pg/mL, 600 pg/mL, 650 pg/mL, 700 pg/mL, 750 pg/mL, 800 pg/mL, 850 pg/mL, 900 pg/mL, 950 pg/mL, 1000 pg/mL, 1050 pg/mL, 1100 pg/mL, 1150 pg/mL, 1200 pg/mL, 1250 pg/mL, 1300 pg/mL, 1350 pg/mL, 1400 pg/mL, 1450 pg/mL, 1500 pg/mL, 1550 pg/mL, 1600 pg/mL, 1650 pg/mL, 1700 pg/mL, 1750 pg/mL, 1800 pg/mL, 1850 pg/mL, 1900 pg/mL, 1950 pg/mL, 2000 pg/mL). The composition may further include about 10 pg/mL to 500 pg/mL of fractalkine and MIP-1 (e.g., 10 pg/mL to 100 pg/mL, 10 pg/mL to 500 pg/mL, or 500 pg/mL to 1000 pg/mL, e.g., 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 150 pg/mL, 200 pg/mL, 250 pg/mL, 300 pg/mL, 350 pg/mL, 400 pg/mL, 450 pg/mL, or 500 pg/mL of fractalkine and MIP-1).

Compositions containing exosome-depleted stem cell secretome-conditioned cell culture medium may further include one or more stem cell activators, such as any amount of an HDAC1 inhibitor (e.g., romidepsin (FK-228), vorinostat, romidepsin, belinostat, panobinostat, VPA, entinostat, curcumin, quercetin, and RG2833), a GSK-3B3 inhibitor (e.g., indirubin-3′-oxime, laduviglusib (CHIR-99821), and KY19382), a NK-1 receptor agonist (e.g., substance P), a FGF protein (e.g., FGF-2), a nutraceutical (e.g., resveratrol, curcumin, and quercetin), Li (e.g., lithium chloride), and/or an NCAM modulator (e.g., N-butylmannosamine or inhibitory nucleic acid molecules that target ST8Sial and/or ST8SiaV) described herein.

### E. Nutraceuticals: Resveratrol, Curcumin, and Quercetin

The present disclosure provides compositions containing about 1 nM to about 10 μM of a nutraceutical (e.g., resveratrol, curcumin, or quercetin) (e.g., 1 nM, 50 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or 10 μM) about 100 nM to about 1 μM (e.g., 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, or 1 μM), about 400 nM to about 600 nM (e.g., 400 nM, 425 nM, 450 nM, 475 nM, 500 nM, 525 nM, 550 nM, 575 nM, or 600 nM), or about 500 nM (e.g., 450 nM, 460 nM, 470 nM, 480 nM, 490 nM, 500 nM, 510 nM, 520 nM, 530 nM, 540 nM, or 550 nM of a nutraceutical (e.g., resveratrol, curcumin, or quercetin)). For example, a composition of the disclosure may contain about 253 nM of resveratrol.

In another example, a composition of the disclosure may contain curcumin at a concentration of about 500 nM (e.g., about 450 nM to about 550 nM or about 475 nM to about 525 nM, e.g., 450 nM, 460 nM, 470 nM, 480 nM, 490 nM, 500 nM, 510 nM, 520 nM, 530 nM, 540 nM, or 550 nM) and resveratrol at a concentration of about 50 nM to about 500 nM (e.g., about 50 nM to about 100 nM, about 75 nM to about 150 nM, about 100 nM to about 200 nM, about 150 nM to about 300 nM, about 200 nM, to about 400 nM, about 240 nM to about 260 nM, about 300 nM to about 500 nM, or about 400 nM to about 500 nM, e.g., 50 nM, 100 nM, 150 nM, 200 nM, 250 nM, 253 nM, 275 nM, 300 nM, 350 nM, 400 nM, 450 nM, or 500 nM). In particular, a composition of the disclosure may contain curcumin at a concentration of about 500 nM and resveratrol at a composition of about 253 nM.

Compositions containing resveratrol, curcumin, and/or quercetin may further include one or more stem cell activators, such as any amount of an HDAC1 inhibitor (e.g., romidepsin (FK-228), vorinostat, romidepsin, belinostat, panobinostat, VPA, entinostat, and RG2833), a GSK-3Bβ inhibitor (e.g., indirubin-3′-oxime, laduviglusib (CHIR-99821), and KY19382), a NK-1 receptor agonist (e.g., substance P), a FGF protein (e.g., FGF-2), Li (e.g., lithium chloride), and/or an NCAM modulator (e.g., N-butylmannosamine or inhibitory nucleic acid molecules that target ST8Sial and/or ST8SiaV) described herein.

F. Neural Cell Adhesion Molecule (NCAM) modulators

The present disclosure provides compositions containing about 1 mM to about 50 mM of N-butylmannosamine (e.g., about 1 mM to about 25 mM, about 5 mM to about 30 mM, about 10 mM, to about 35 mM, about 15 mM, to about 40 mM, about 20 mM to about 45 mM, or about 25 mM to about 50 mM, e.g., about 1 mM, about 2 mM, about 3 mM, about 4 mM, about 5 mM, about 6 mM, about 7 mM, about 8 mM, about 9 mM, about 10 mM about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, or about 50 mM of N-butylmannosamine).

In another example, a composition of the disclosure may contain an siRNA, ASO, shRNA, miRNA, or dsRNA that target ST8Sial and/or ST8SiaV (e.g., Table 3) and reduces expression (e.g., mRNA and/or protein expression levels) of ST8Sial and/or ST8SiaV in a cell of the subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

Compositions containing an NCAM modulator may further include one or more stem cell activators, such as any amount of an HDAC1 inhibitor (e.g., romidepsin (FK-228), vorinostat, romidepsin, belinostat, panobinostat, VPA, entinostat, curcumin, quercetin, and RG2833), a GSK-3B3 inhibitor (e.g., indirubin-3′-oxime, laduviglusib (CHIR-99821), and KY19382), a NK-1 receptor agonist (e.g., substance P), a FGF protein (e.g., FGF-2), a nutraceutical (e.g., resveratrol, curcumin, and quercetin), and/or Li (e.g., lithium chloride) as described herein.

The following Examples are provided to illustrate enablement and utility of the embodiments herein and are not to be limiting.

### EXAMPLES

A series of assays quantified various functional aspects of stem cell activation including proliferation, migration, epigenetic reprogramming, and stem cell secretome analysis. These assays were validated through comparison to prior results. Results show that Li and VPA enhance proliferation and migration of mesenchymal stem cells (MSCs) and neural stem cells (NSCs) in a dose-dependent manner. Cell migration by valproic acid (VPA) was inhibited by blockage of C—X—C motif chemokine receptor 4 (CXCR4) and lithium (Li)-induced cell migration was inhibited by blockage of matrix metallopeptidase 9 (MMP-9), suggesting their involvement in the mechanism of stem cell activation. Additional molecular mechanisms are possible. The data suggests a role for epigenetic modulation in stem cell activation.

### Example 1: Expansion of Cell Lines for Use in Cell-Based Assays

Native human umbilical cord-derived MSCs (Vitro Biopharma Cat. No. SC00A1), human pancreatic fibroblasts (Vitro Biopharma, Cat. No. SC00AS), colorectal cancer-associated fibroblasts (Vitro Biopharma, Cat. No. CAF05), and pancreatic stellate cancer-associated fibroblasts (Vitro Biopharma, Cat. No. CAF08) were plated at 7500 cells/cm² and grown to 90% confluency in T-25 tissue culture (TC) flasks (BD Falcon, Cat. No. 353108) in MSC-GRO™ low serum, complete medium (Vitro Biopharma Cat. No. SC00B1). Neural stem cells (Vitro Biopharma, Cat. No. SC00A1-NSC), were similarly cultured except that neural MSC-GRO™ medium (Vitro Biopharma, Cat. No. NSCB1) and laminin-coated T-25 flasks (Corning BioCoat, Cat. No. 354533) were used. Cultures were maintained in a humidified chamber equilibrated with 5% CO₂, 1% O₂, balance N₂ at 37° C. Cells were detached using ACCUTASE™ (Innovative Cell Technologies Inc., Cat No. AT-104) and collected by centrifugation (450×g) for 7 minutes. Following aspiration, the cell pellet was resuspended in 1 mL PBS and cells were counted using a Beckerman-Coulter Z2 particle counter (range 10 μm-30 μm).

### Example 2: Stem Cell Migration Assay

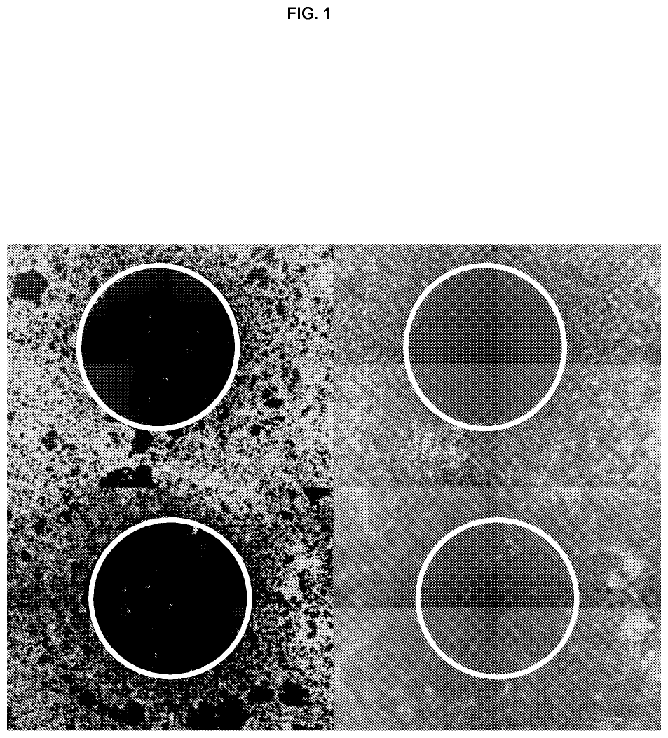

In order to investigate the effects of drugs and nutraceuticals on human stem cell migration, a stem cell-based assay of migration was developed and validated. FIG. 1 shows the basic components of the assay and shows the effects of substance P, a well-known inducing agent of cell migration. FIG. 1 is a microphotograph depicting cell migration using fluorescent readout and cell tracker green as a fluorescent marker of human UC-MSCs. These cell images show fluorescent human MSCs at the beginning of the assay (left panel) of the Control vs. activating agent (substance P at 3.7 nM) and 24 hours later (right panel). MSCs migrated to the cell-free center of the well and also filled open areas in other regions of the culture as a result of substance P exposure (lower right panel) but did not similarly migrate in its absence (upper right panel). The EC₅₀ was determined as a measure of effectiveness of activating agents. Initial kinetic data allowed determination of the optimal assay parameters for further experiments. An initial analysis of the cell free zone using Image J software was used to screen concentrations of activating agents for dose-response determination in further experiments. It was determined that MSCs plated at 25,000/well, incubated for 24 hours and then exposed to appropriate activator concentrations gave optimal results.

FIG. 1 shows the results of testing the effect of 3.7 nM substance P on the migration of human UC-MSCs. In the absence of substance P (upper panels), no migration was detected into the cell-free zone created by culturing cells in the presence of an occluding plug that prevented cell attachment in the center of the well during 24 hours of culture and this was a consistently observed result under control conditions, i.e., no activator. On the other hand, 3.7 nM substance P-induced MSC migration into the cell-free center of the well and increased fluorescence within the original, cell containing region of the well (FIG. 1, lower two panels). Analysis of fluorescence within the cell-free region at the center of the well was used as a quantitative measure of cell migration as described below.

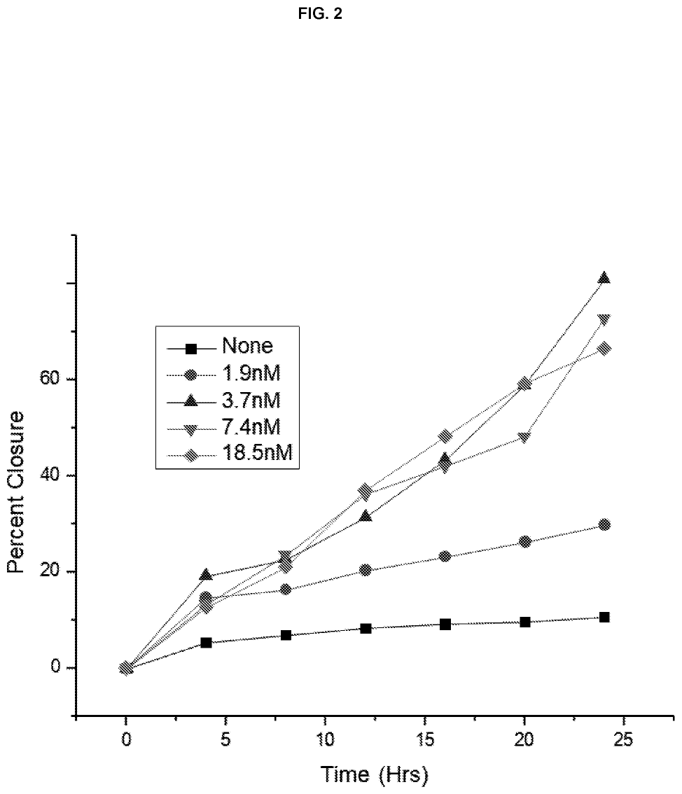

FIG. 2 is a line graph representation illustrating percent closure as a function of time during live-cell data acquisition at different dosages of substance P. FIG. 2 shows the dose-response of substance P-induced migration of cord blood mesenchymal stem cells (CB-MSCs). Percent closure is shown under control conditions (no substance P) and with increasing concentrations of substance P as a function of time during the 24-hour period of live cell analysis. No migration is seen without substance P, while migration increased in a dose-dependent manner by exposure to substance P from 0 to 18.5 nM. Migration exhibited saturation at higher substance P concentrations.

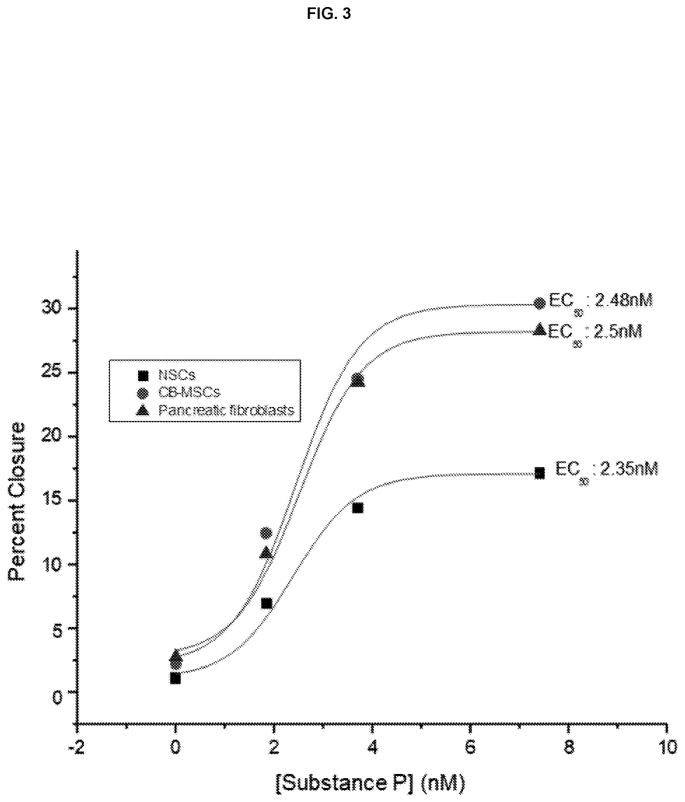

FIG. 3 is a line graph representation illustrating migration of various human cell lines exposed to substance P. FIG. 3 shows the dose response relationship for substance P-induced migration of MSCs, a primary human pancreatic cell line and NSCs as well. The data shows the dose-response curve of MSCs (circles), human primary pancreatic fibroblasts (triangles), and human NSCs (squares) together with EC₅₀ values. Percent closure was determined at 24 hours. By fitting the data to a sigmoidal curve, EC₅₀ values of 2.48 nM, 2.5 nM, and 2.35 nM were calculated for MSCs, human pancreatic fibroblasts, and NSCs, respectively. The EC₅₀ obtained for human pancreatic fibroblasts, i.e., 2.5 nM, compares well with a prior study of human fibroblast migration induced by substance P of 2.2 nM using a suspension culture system to measure cell migration. Human fibroblast migration was mediated through the neurokinin-1 (NK-1) receptor, since NK-1 receptor agonists mimicked substance P and NK-1 receptor antagonists blocked substance P induction of fibroblast migration. Fibroblast migration induced by substance P is an important response to injury in addition to the induction of MSC migration. (Parenti, A., et al., Naunyn Schmiedeberg's Arch Pharmacol 353:475-481, 1996). Since the EC₅₀ for substance P is comparable to prior results, these results provide validation support for the cell migration assay.

The cell migration assays described above were set up as follows: One million cells/cell line were resuspended in 10 mL MSC-GRO™ serum free, quiescent medium (Vitro Biopharma Cat. No. SC00B17) containing 5 pg/mL mitomycin C (Sigma, Cat. No. M4287) to inhibit proliferation and incubated for 2 hours at room temperature with end-to-end agitation at 7 RPM. In some experiments, curcumin was used at 1 μM to block proliferation. Cells were centrifuged (450×g) for 7 minutes, washed with phosphate buffered saline (PBS) and then resuspended in 1 mL MSC-GRO™ low serum, complete 20 medium (Vitro Biopharma Cat. No. SC00B1) and plated at 25,000 cells/well in black 96 well, TC-coated cell culture plates, (ThermoScientific, Cat. No. 165305) containing cell seed-stoppers (Platypus, Cat. No. CMAUFL4) to form a cell free zone at the center of the well and incubated in 5% CO₂, 1% O₂, 94% N₂ at 37° C. in a humidified chamber for 24 hrs. Plates used for culture of NSCs, were first treated with 10 μg/mL fibronectin (Sigma, Cat. No. F0556) for 2 hours at 37° C. Following washout with PBS (3×), cell seed stoppers were inserted, NSCs were plated at 25,000/well and incubated in 5% CO₂, 1% O₂, 94% N₂ at 37° C. in a humidified chamber for 48 hrs. For studies of the effects of CXCR4 and MMP-9 inhibition, following 24 hours of cell culture, appropriate wells were dosed with 15 μM GM6001 and 20 μM AMD3100 for 6 hours. Cells were washed once with PBS then incubated in serum free, MSC-GRO™ (Vitro Biopharma Cat. No. SC00B17) containing 5 μM Cell Tracker Green CMFDA (Molecular Probes, Cat. No. C7025) at 37° C. for 30 minutes. The wells were then washed with serum free, MSC-Gro™ (Vitro Biopharma Cat. No. SC00B17) and incubated for 30 minutes at 37° C. Cells were washed once with PBS and this was replaced with MSC-GRO™ serum free, quiescent medium (Vitro Biopharma Cat. No. SC00B1I7) containing different concentrations of activating agents. Substance P was from Tocris Bioscience, (Cat. No. 1156) and curcumin from Santa Cruz Biotechnology (Cat. No. SC-200509A), VPA from Reagents Direct (Catalog Number 25-B43) and lithium chloride from Sigma Chemical Co. (Catalog number L4408). A TOPSEAL™ (PerkinElmer, Cat. No. 6050195) covered the plate for live-cell imaging in a BioTek Cytation3 Imaging Reader. Kinetic data was acquired every 2 hrs for 24 hrs using a GFP filter and bright field data acquisition. The gas phase throughout the acquisition of kinetic data was 5% O₂, 5% CO₂ with the balance nitrogen maintained by a BioTek CO2/O2 gas controller. Images were saved as TIFF files to calculate percent closure using imaging data.

### Example 3: Effect of Curcumin on Human MSCs/NSCs Migration

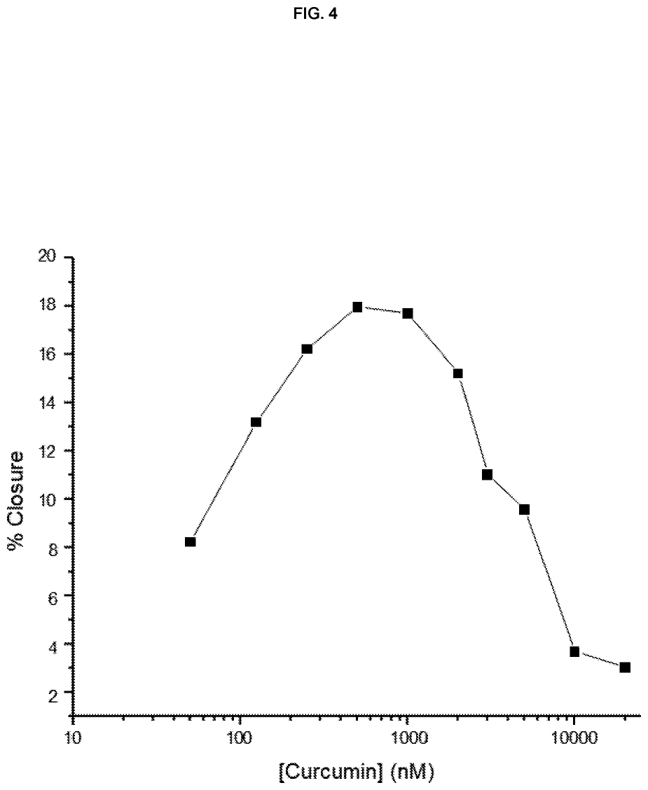

Because curcumin had been previously shown to enhance rat NSC proliferation and enhanced recovery of spinal cord injury by use of NSCs pre-treated with curcumin (Ormond, D R, et al., PLoS ONE 9: e88916, 2014), the effects of curcumin were tested on MSC migration and the results are shown in FIG. 4, which is a line graph representation illustrating less than 1 μM curcumin induced migration of human MSCs and 1 to 10 μM curcumin blocked migration due to apparent toxicity. Percent closure was determined after a 36-hour run period. At concentrations less than 1 μM, curcumin induced migration with an apparent EC₅₀ of 250 nM and at concentrations greater than 1 μM, curcumin blocked migration by apparent toxicity indicated by reduced cell number with an estimated LD₅₀ of 3 μM. These results also compare with prior studies (Ormond, D R, et al, PLoS ONE 9: e88916, 2014) providing further validation support for the cell migration assay. In addition to inducing proliferation of NSCs, curcumin also induces migration of MSCs and is toxic to MSCs at concentrations greater than 1 μM. Curcumin is thus emerging as a natural substance that serves as an activator of adult stem cells including MSCs and NSCs through increased stem cell proliferation and migration.

### Example 4: Effects of Lithium and VPA on Migration of Human Stem Cells

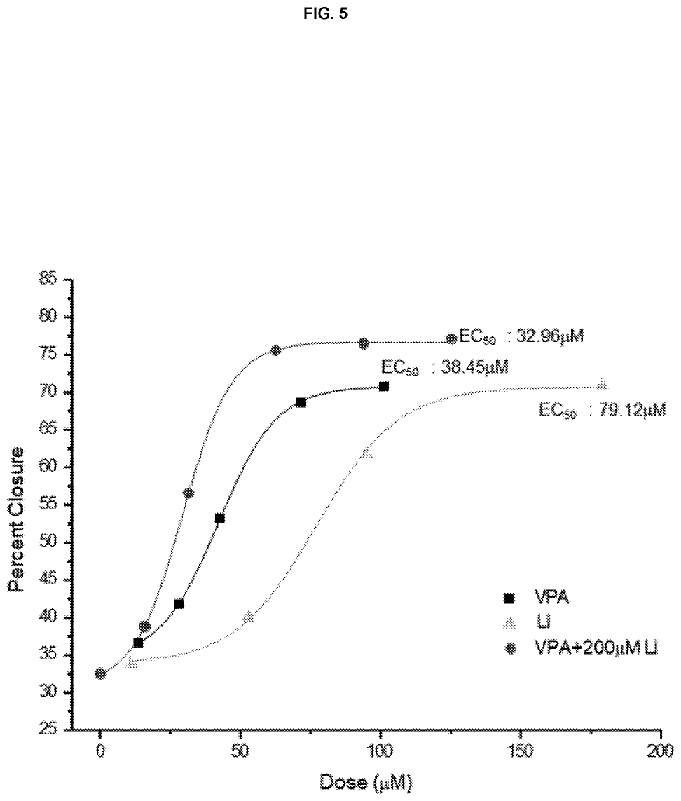

FIG. 5 is a line graph representation illustrating migration of MSCs induced by exposure to lithium alone (triangles), VPA alone (squares) and VPA in the presence of 200 μM lithium (circles). Percent closure is plotted as a function of dose and the data was modeled by sigmoidal curve fitting to calculate EC₅₀ values. FIG. 5 shows lithium-induced MSC migration with a calculated EC₅₀ of 79.12 μM and maximal migration at 200 μM lithium. VPA also induced MSC migration with an EC₅₀ of 38.45 μM with maximum closure at 100 μM. Since maximal lithium-induced closure occurred at 200 μM, the closure induced by increasing VPA concentrations in 200 μM lithium was then investigated. The results showed a lower EC₅₀ than that observed with VPA only, i.e., 32.96 μM suggesting a synergistic effect of Li-VPA on MSC migration.

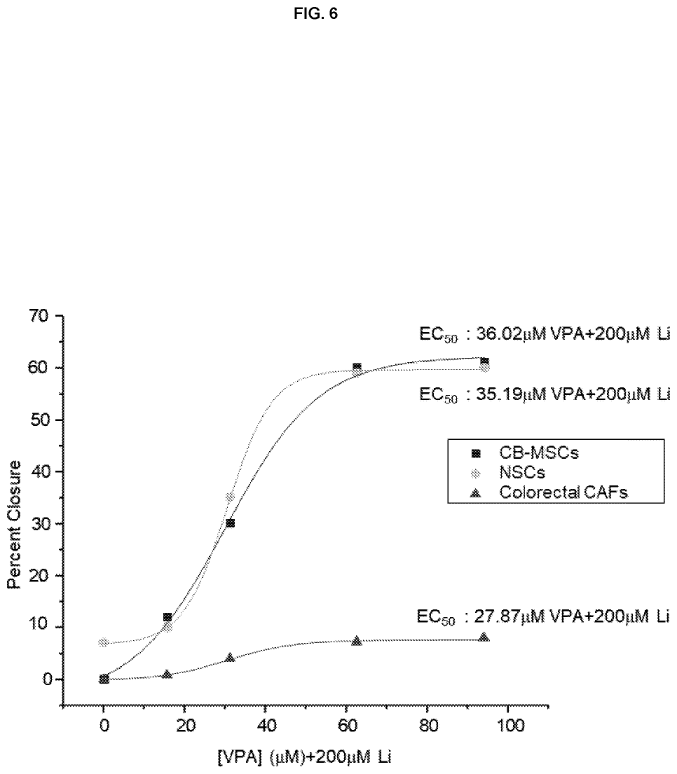

The effect of Li-VPA on cell migration of different cell lines was determined; the results are shown in FIG. 6, which is a line graph representation illustrating migration of CB-MSCs, NSCs and colorectal CAFs by induced by increasing VPA concentration in 200 μM lithium. Percent closure is plotted as a function of VPA in medium containing 200 μM lithium. Percent closure is plotted as a function of dose and the data was modeled by sigmoidal curve fitting to calculate EC₅₀ values.

Migration of MSCs (squares), NSCs (circles) and colorectal CAFs (triangles) are shown together with calculated EC₅₀ values. While increasing VPA in 200 μM lithium showed similar kinetics between MSCs and NSCs, EC₅₀ was 36.02 and 35.19 μM, colorectal CAFs did not migrate to the same extent as MSCs and NSCs and similar data was obtained for other CAFs. Thus, while MSCs and NSCs are robustly induced to migrate by Li-VPA, CAFs do not similarly migrate.

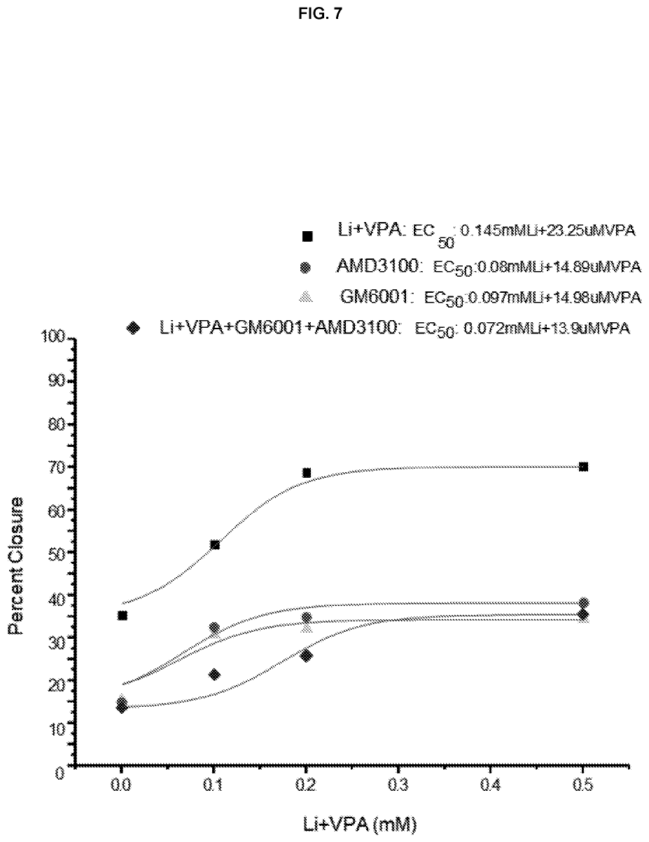

The molecular mechanisms of the effect of lithium and VPA were then investigated on stem cell migration. Since a prior report suggested that VPA up-regulated CXCR4, a critical chemokine receptor involved with cellular mobility, and that lithium up-regulated MMP-9 (Tsai, L K, et al., Stroke 42(10): 2932-2939, 2011), the effects of known inhibitors of CXCR4 and MMP-9 on the migration of MSCs were determined and the results are shown in FIG. 7, which is a line graph representation illustrating migration of human MSCs exposed to a combination of lithium and VPA with and without inhibition of MMP9 and CXCR4. The data shows the dose response curve of MSCs treated with Li and VPA alone (squares), treated with Li and VPA and with inhibition of CXCR4 by AMD3100 (circles), treated with Li and VPA and inhibition of MMP9 by GM6001 (triangles), and treating with Li and VPA while inhibiting both CXCR4 and MMP9 (diamonds). Percent closure is plotted as a function of dose. The results indicate that the CXCR-4 inhibitor, AMD 3100, blocked the VPA-induced CB-MSC migration and that GM 6001, a competitive inhibitor of MMP-9, blocked lithium induced CB-MSCs. Also, in the presence of both AMD 3100 and GM6001, MSC migration induced by Li-VPA was also blocked. These results suggest that CXCR4 and MMP-9 are molecular components of the VPA and lithium-induced migration of MSCs thus confirming and extending prior results of Tsai, L K, et al., Stroke 42:2932-2939, 2011.

### Example 5: Effects of FGF-2, Lithium, and VPA on Stem Cell Proliferation

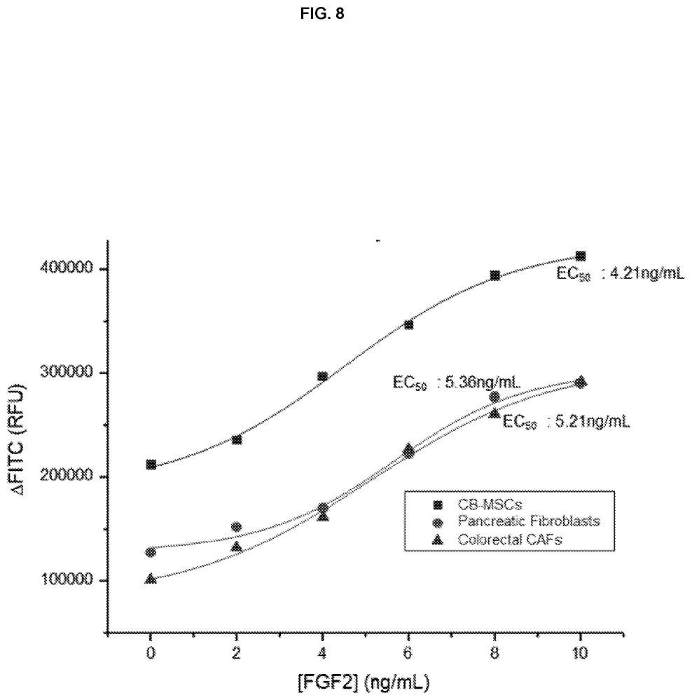

The effects of fibroblast growth factor 2 (FGF-2), lithium, and VPA on stem cell proliferation were then investigated, using an assay based on Presto Blue, a fluorescent marker of cellular reduction. FIG. 8 is a line graph representation illustrating proliferation of different cell lines induced by increasing concentrations of FGF-b (FGF-2) after a 5 day exposure. Relative fluorescent units were measured using a FITC filter on a Modulus Microplate Reader and plotted as a function of FGF-2. The data were modeled by sigmoidal curve fitting to calculate EC₅₀ values. FIG. 8 shows the effect of FGF-2 on the proliferation of different cell lines including CAFs, primary fibroblasts and MSCs. Proliferation of these mesenchymal cells was stimulated by recombinant human FGF-2 with EC₅₀ values in the range of 3 ng/mL to 6 ng/mL with maximal proliferative responses at about 10 ng/mL. Since these results are comparable to previous studies (Lee, T H, et al, Biochem Cell Biol. 26: 1-8, 2015), these data provide validation support for the cell-based proliferation assay.

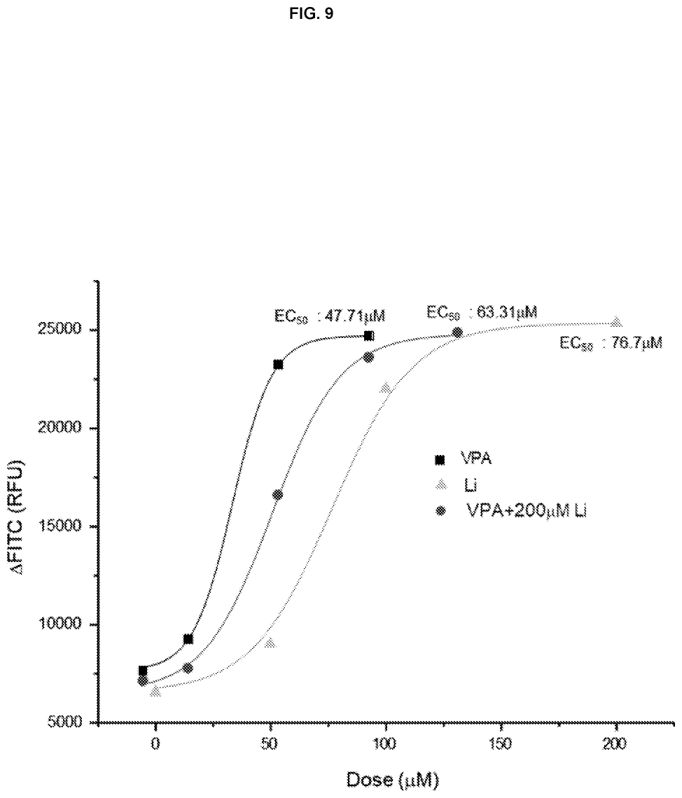

The proliferative effects of lithium and VPA were compared, either alone or in combination on MSC proliferation, and the results are shown in FIG. 9, which is a line graph representation illustrating proliferation of MSCs induced by lithium, VPA and VPA in 200 μM lithium after a 5 day exposure. Relative fluorescent units were measured using a FITC filter on a Modulus Microplate Reader and plotted as a function of FGF-2. The data was modeled by sigmoidal curve fitting to calculate EC₅₀ values. The EC₅₀ value was 76.7 μM for lithium and this was comparable to the lithium effect on MSC migration (FIG. 5). The EC₅₀ for VPA was 47.71 μM and in the presence of 200 μM lithium, the EC₅₀ for VPA was 63.31 μM. While MSC migration showed an apparent synergy with combined Li-VPA this does not appear to be the case for MSC proliferation, since larger VPA dosages were needed for equivalent MSC proliferation in the presence of 200 μM lithium.

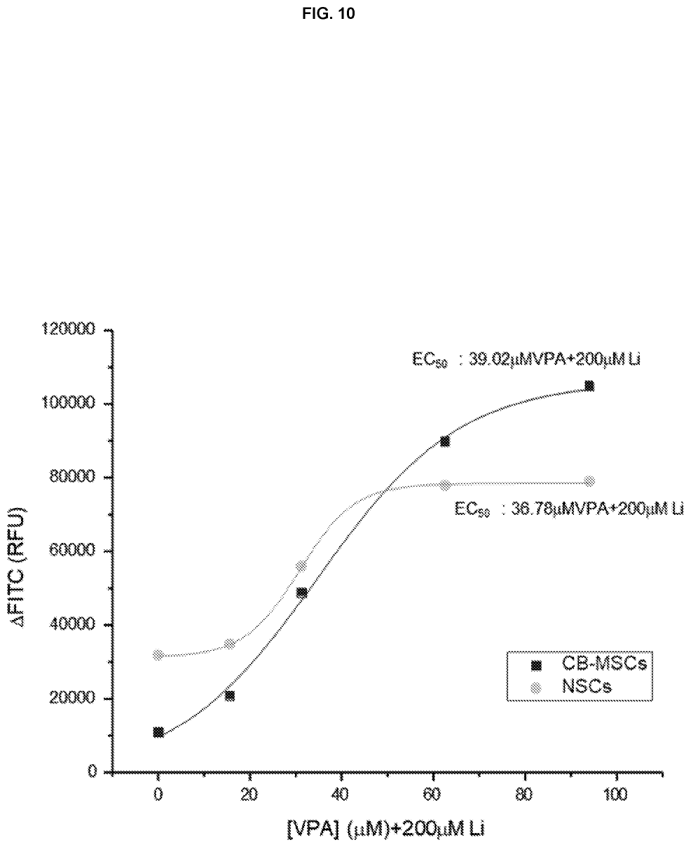

The proliferative responses of MSCs and NSCs were compared to Li-VPA and the results are shown in FIG. 10, which is a line graph representation illustrating proliferation of MSCs and NSCs as a function of increasing VPA in 200 μM lithium after a 5 day exposure. Relative fluorescent units were measured using a FITC filter on a Modulus Microplate Reader and plotted as a function of FGF-2. The data were modeled by sigmoidal curve fitting to calculate EC₅₀ values. While both NSCs and MSCs exhibited comparable EC₅₀, 36.78 and 39.02 μM, respectively, the extent of proliferation is reduced in NSCs compared to MSCs and this may reflect intrinsic proliferative capacity. Also, since the cell migration results yielded similar results, both proliferation and migration of MSCs and NSCs may be activated at similar reduced dosage when lithium and VPA are administered in combination. Thus, in the presence of 200 μM lithium, VPA is 50% effective in inducing cell proliferation and migration at 35 to 39 μM (mean 37 μM).

### Example 6: Effects of VPA and Lithium on Stem Cell Gene Expression

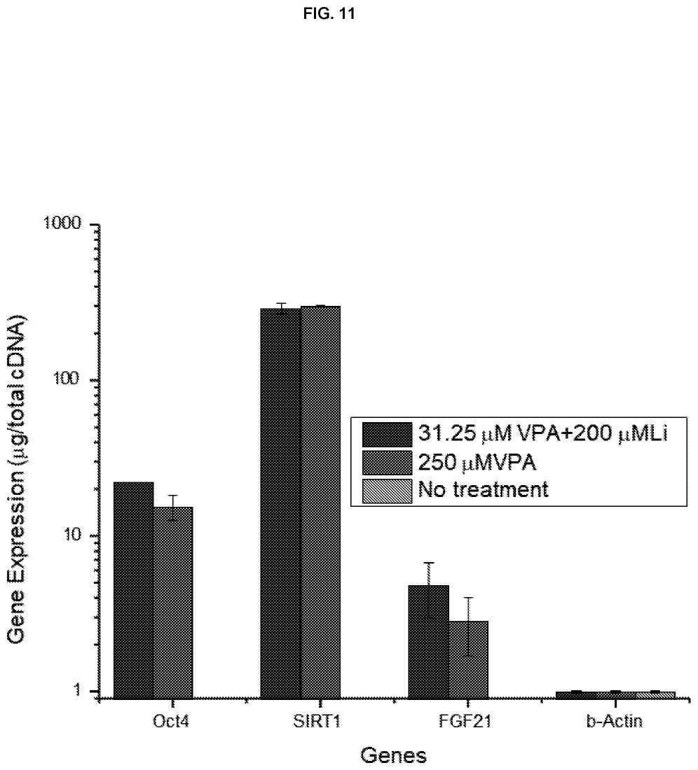

In FIG. 11, results of gene expression analysis are shown following the exposure of MSCs to VPA. FIG. 11 is a bar graph representation illustrating PCR used to measure target genes known to be subject to epigenetic regulation by HDAC inhibitors. The graph shows the result of human MSCs treated with VPA alone or in combination with lithium. The expression of Oct 3/4, a well-known pluripotency gene, was increased about 20-fold compared to untreated human MSCs. The expression of SIRT-1 was highly elevated compared to untreated MSC by ˜300-fold without differences between treatment with either VPA or lithium-VPA. The expression of FGF-21 was also elevated by 3 to 5-fold and its expression was higher in MSCs treated with Li-VPA, although this increase with not significant. All gene expression was normalized to untreated MSCs.

Beta actin was measured as a house-keeping gene. The graph shows the result of gene expression analysis of human MSCs treated with VPA only or VPA+ lithium. Gene expression was quantified by determining the amount of gene-specific DNA/total cDNA. Data are mean+/−SD of 4 replicates. These results thus show increased expression of Oct 3/4, SIRT-1 and FGF-21 as a result of exposure to VPA or Li-VPA using 200 μM lithium and 31.25 μM VPA.

Native human umbilical cord-derived MSCs (Vitro Biopharma Cat. No. SC00A1) were expanded from cryopreservation in a T-25 TC-coated flasks (BD Falcon, Cat. No. 353108) in MSC-GRO™ low serum, complete medium (Vitro Biopharma Cat. No. SC00BI). Cells were subcultured and counted on a Beckerman-Coulter Z2 particle counter (range 10 μm-30 μm). Cells were plated at 10,000/cm² a TC-coated Greiner Bio-One T75 flask and maintained in MSC-GRO™ serum free, complete medium (Vitro Biopharma Cat. No. SC00B3) in a reduced 02 environment (1% O₂, 5% CO₂, 94% N₂) at 37° C. in a humidified chamber. The MSCs were treated continuously for up to 2 weeks. Cultures were fed every three days. Cells were harvested using ACCUTASE™ (Innovative Cell Technologies Inc., Cat No AT-104) and centrifuged (450×g) for 7 minutes. Cell supernatant was aspirated off and cells were resuspended in 1 mL PBS and counted on a Beckerman-Coulter Z2 particle counter (range 10 μm-30 μm). Total RNA was extracted using RNeasy Mini Kit (Qiagen Cat. No. 74104). RNA was quantified using an absorbance measurement at 260 nm. RNA was converted to cDNA using Quantitect Reverse Transcription Kit (Qiagen Cat. No. 205310) in a thermocycler. RNA was incubated in the gDNA elimination reaction for 2 minutes at 42° C. then incubated in the reverse-transcription master mix for 15 minutes at 42° C. Immediately after, it was incubated at 95° C. for 3 minutes for inactivation. cDNA was sent to an outside lab (CU-Anschutz Metabolic Laboratory) for q-PCR to detect relative or absolute gene expression levels. cDNA was diluted 1:5 and iTaq Universal Supermix fluorescent probe (BioRad Cat. No. 172-5120) used to detect the threshold cycle (Ct) during PCR. Dilution factors and cDNA concentrations were calculated into recorded values then normalized to untreated hMSCs (Vitro Biopharma Cat. No. SC00A1).

### Example 7: Secretion of Cytokines from MSCs

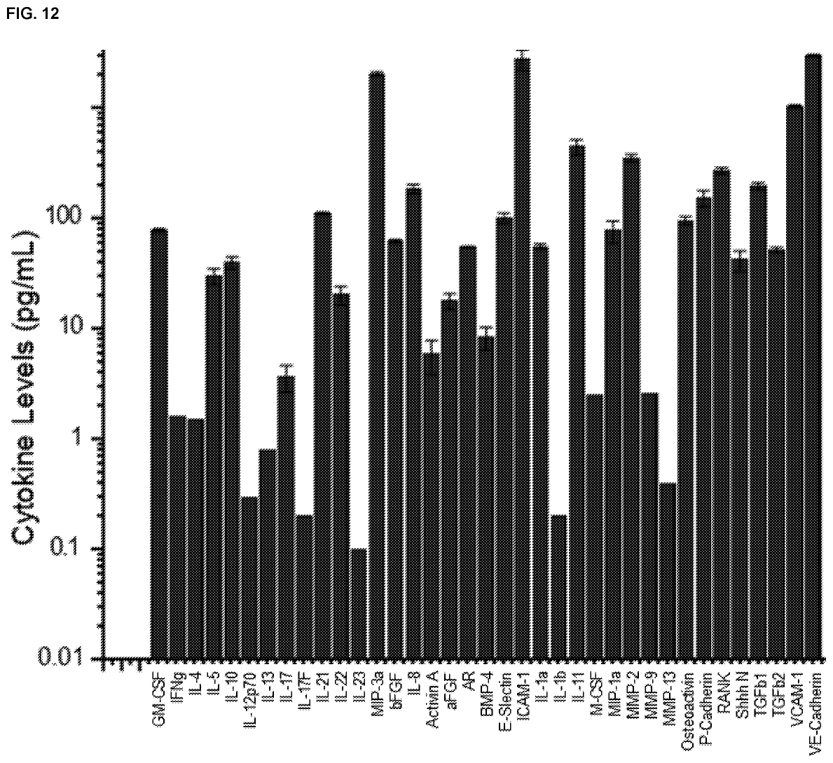

Preliminary experiments were performed concerning the composition of cytokines within the soluble factors secreted from stem cells and the results are shown in FIG. 12, which is a bar graph representation illustrating cytokine levels in cell culture media exposed to MSCs for 24 days and determined by microarray analysis. Conditioned media was analyzed using an inflammation microarray, Th17 microarray and a bone metabolism array. Results show an increase in inflammatory cytokines as well as adhesion factors. With a substantial increase in the content of macrophage inflammatory protein (MIP)-3 alpha (MIP-3a), Intercellular Adhesion Molecule 1 (ICAM-1), IVCAM-1 and VE-Cadherin within the conditioned medium, this suggests the well-known immunosuppressive role of MSCs.

Native human umbilical cord-derived MSCs (Vitro Biopharma Cat. No. SCOOA1) were plated at 1,000/well in a tissue cultured 6-well plate (BD Falcon, Cat Number 353046) in MSC-GRO™ low serum, complete medium (Vitro Biopharma Cat. No. SC00BI). Cells were continuously grown for a period of 30 days. Conditioned media was collected at day 3, 6, 12, 18, and 24. Multiple microarrays were run using conditioned media for cytokine secretion determination after a continuous 24 day culture period. An inflammation microarray (Ray Biotech, Cat. No. QAH-INF-3), bone metabolism microarray (Ray Biotech, Cat. No. QAH-BMA-1) and a Th17 microarray (Cat. No. QH-TH17-1) were used to analyze the conditioned media. A laser scanner (Molecular Probes, Genepix 4000B) was used to measure the fluorescent signals of each microarray.

### Example 8: Effects of Nutraceuticals on Stem Cell Gene Expression

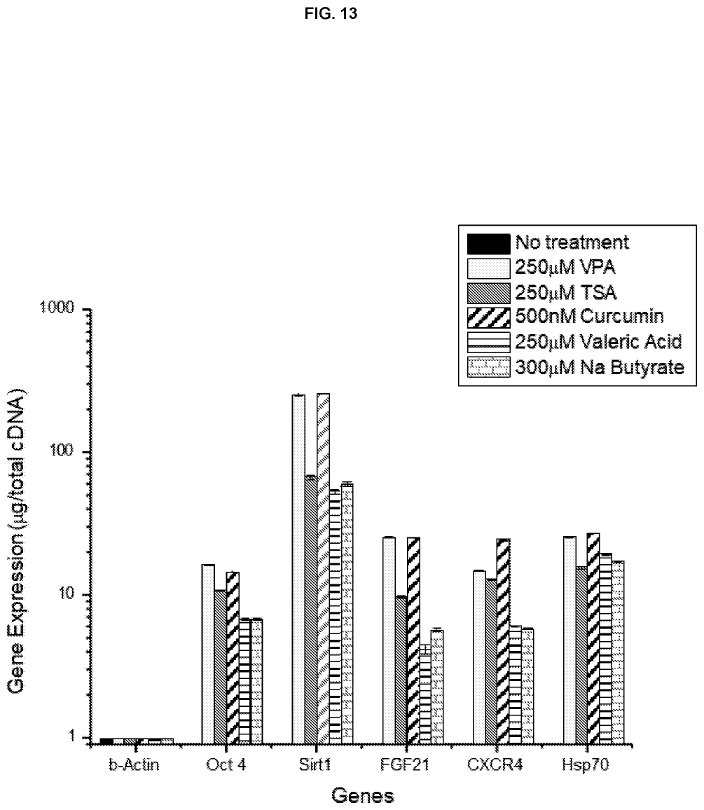

In FIG. 13, results of gene expression analysis are shown following the exposure of MSCs to VPA and various nutraceuticals known to exhibit epigenetic effects. The expression of various target genes as determined by qPCR is shown as compared to the house-keeping gene, beta-actin. The expression of Oct 3/4, a well-known stem cell pluripotency gene, was increased about 20-fold compared to beta-actin in human MSCs by VPA and curcumin while TSA, valeric acid, and Na butyrate resulted in less expression activation. The expression of SIRT-1 was highly elevated by at least 200-fold without differences between treatment with either VPA or Curcumin while TSA, valeric acid and Na butyrate were significantly less effective. Similar yet lower expression levels of fibroblast growth factor 21 (FGF-21), CXCR4 and heat shock protein 70 (Hsp70) were observed (maximum of 25-fold) with VPA and curcumin showing nearly equivalent expression increases and lower levels with TSA, valeric acid and Na butyrate. Curcumin at 500 nM increased Oct 3/4 expression at least 10-fold, increased CXCR-4 expression at least 20-fold, increased Hsp70 expression at least 20-fold and also increased FGF-21 at least 20-fold compared to untreated cells at statistically significant levels. All gene expression was normalized to that of untreated MSCs. Beta-actin was used as a housekeeping gene.

Gene expression was quantified by determining the amount of gene-specific DNA/total cDNA. Data are mean+/−S.D. of 4 replicates. These results thus show highest increased expression of Oct 3/4, SIRT-1, FGF-21, CXCR4, and Hsp70 as a result of exposure to 250 μM VPA and 500 nM Curcumin, the later likely due to its HDAC inhibitory effects (Soflaei S S, et. al, Curr Pharm Des. 2018; 24(2):123-129) although it has other epigenetic effects as well. Regardless of the precise mechanisms, curcumin showed equivalent or increased expression of Oct 3/4, SIRT-1, FGF-21, CXCR4 & Hsp70 as compared to VPA.

Native human umbilical cord-derived MSCs (Vitro Biopharma Cat. No. SCOOA1) were expanded from cryopreservation in a T-25 TC-coated flasks (BD Falcon, Cat. No. 353108) in MSC-GRO™ low serum, complete medium (Vitro Biopharma Cat. No. SC00B1). Cells were subcultured and counted on a Beckerman-Coulter Z2 particle counter (range 10 μm-30 μm). Cells were plated at 10,000/cm² a TC-coated Greiner Bio-One T75 flask and maintained in MSC-GRO™ serum free, complete medium (Vitro Biopharma Cat. No. SC00B3) in a reduced 02 environment (1% O₂, 5% C O₂, 94% N₂) at 37° C. in a humidified chamber. The MSCs were treated continuously for up to 2 weeks. Cultures were fed every three days. Cells were harvested using ACCUTASE™ (Innovative Cell Technologies Inc., Cat No AT-104) and centrifuged (450×g) for 7 minutes. Cell supernatant was aspirated off and cells were resuspended in 1 mL PBS and counted on a Beckerman-Coulter Z2 particle counter (range 10 μm-30 μm).

Total RNA was extracted using RNeasy Mini Kit (Qiagen Cat. No. 74104). RNA was quantified using an absorbance measurement at 260 nm. RNA was converted to cDNA using Quantitect Reverse Transcription Kit (Qiagen Cat. No. 205310) in a thermocycler. RNA was incubated in the DNA elimination reaction for 2 minutes at 42° C. then incubated in the reverse-transcription master mix for 15 minutes at 42° C. Immediately after, it was incubated at 95° C. for 3 minutes for inactivation. cDNA was sent to an outside lab (CU-Anschutz Metabolic Laboratory) for q-PCR to detect relative or absolute gene expression levels. cDNA was diluted 1:5 and iTaq Universal Supermix fluorescent probe (BioRad Cat. No. 172-5120) used to detect the threshold cycle (Ct) during PCR. Dilution factors and cDNA concentrations were calculated into recorded values then normalized to untreated hMSCs (Vitro Biopharma Cat. No. SC00A1).

### Example 9: Effects of Nutraceuticals on Stem Cell Migration

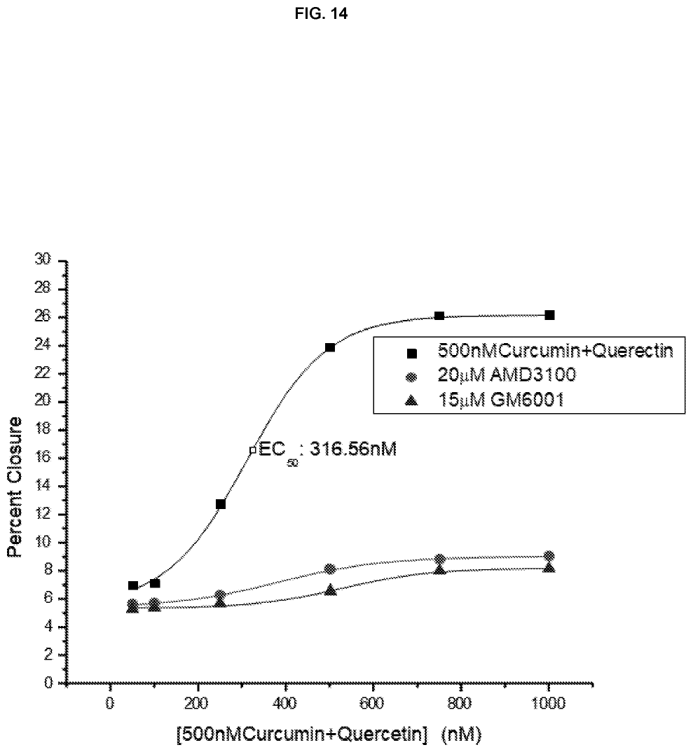

FIG. 14 shows the migration of MSCs induced by exposure to 500 nM curcumin and variable concentrations of quercetin (squares) alone, or in the presence of the CXCR4 inhibitor (AMD3100) (circles) and the MMP9 inhibitor (GM6001) (triangles). Percent closure is plotted as a function of dose and the data was modeled by sigmoidal curve fitting to calculate EC₅₀ values. FIG. 14 shows curcumin and quercetin induced MSC migration with a calculated EC₅₀ of 316.56 nM quercetin and maximal migration of the combination of curcumin and quercetin. The CXCR4 inhibitor, AMD3100, blocked MSC migration as did the MMP9 inhibitor, GM6001, suggesting a similar molecular mechanism for the migration of MSCs by curcumin & quercetin as for stem cell migration induced by VPA-Li.

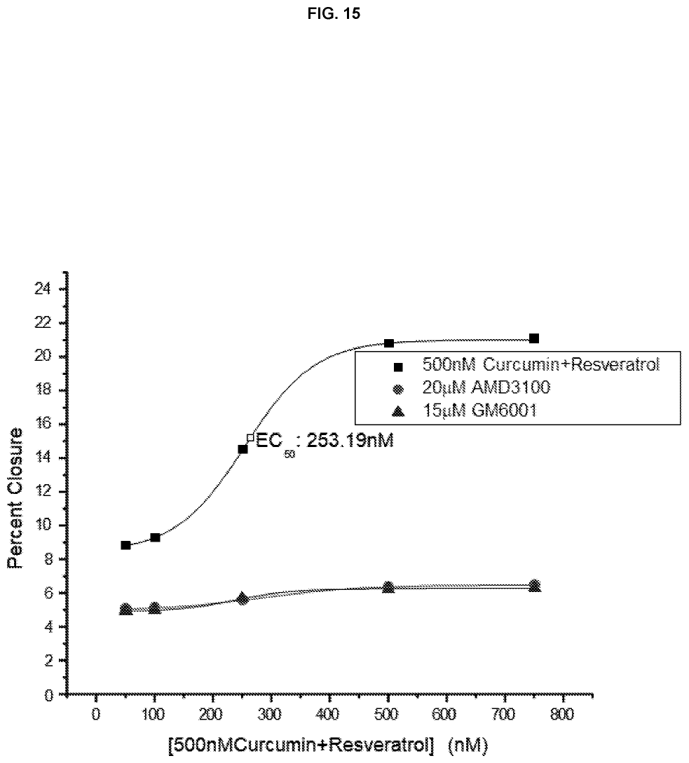

FIG. 15 shows the migration of MSCs induced by exposure to 500 nM curcumin and variable concentrations of resveratrol (squares) alone, or in the presence of the CXCR4 inhibitor (AMD3100) (circles) and the MMP9 inhibitor (GM6001) (triangles). Percent closure is plotted as a function of resveratrol dose (nM) and the data was modeled by sigmoidal curve fitting to calculate EC₅₀ values. These results show that curcumin and resveratrol-induced MSC migration with a calculated EC₅₀ of 253.19 nM resveratrol and maximal migration of the combination of curcumin and resveratrol. The CXCR4 inhibitor, AMD3100, blocked MSC migration as did the MMP9 inhibitor, GM6001 suggesting a similar molecular mechanism for the migration of MSCs by curcumin and resveratrol as for stem cell migration induced by VPA-Li.

### Example 10: Extraction of Mononuclear Cells from Human Umbilical Cord

### Methods:

Human umbilical cord was obtained from CryoPoint, Brownsville, IN under informed consent from the donor. It was transported in saline and maintained at 4° C. during transport. The umbilical cord was micro-dissected into 1-2 cm² pieces in a petri dish containing PBS within a biological safety cabinet. All subsequent procedures occurred in a sterile environment. An enzymatic digestion mixture was prepared in 0.2 PZ U/mL Collagenase NB 6 GMP grade, Serva Chemicals, in HEPES-buffered saline at 2.5 mL/gm umbilical cord tissue. This mixture was incubated at 37° C. in an end-over-end rotator at 25 RPM. Following incubation, cells and tissue were separated using a Buchner funnel (90 to 130-micron pore size) following washout of residual cells, with 3×20 mL PBS. Cells were pelleted by centrifugation at 440×G for 15 minutes and re-suspended in 10-20 mL PBS for cell counting including total cell count (10 to 40 micron) and acridine-orange, propidium iodide using the Countess II instrument (Fisher Scientific). The mononuclear cells (MNCs) were re-suspended at 106 MNC/mL in CryoStore 2 cryopreservation medium (BioLife Solutions, Catalog Number 202102) and cryopreserved using controlled rate freezing from room temperature to −80° C. at ˜1° C./minute, followed by storage in liquid nitrogen.

### Results

The above procedures yielded a total cell count of 530 million, consisting of 13 million MNCs at 47.3% viability.

### Expansion of MSCS

### Methods

Pass 0 cultures, i.e., initial passage following collagenase digest, were cultured at 15K to 20K total cells/cm² in T-75 TC-coated tissue culture flasks (Falcon, Catalog Number 353136) in MSC cell culture medium (MSC-GRO™, Vitro Biopharma catalog number SC00B4-3, Clinical Grade Humanized, Serum-free medium, (supplemented to 5% Human serum AB, Golden West Biologicals, Temecula, CA) plus 1× penicillin/streptomycin (Sigma, catalog number P4383). The cultures were maintained in a tri-gas incubator (HerraCell, 240i; Fisher Scientific) set to 5% 02 and 5% CO₂. These cultures were monitored at 4 to 5-day intervals for colony formation and expansion. Pass 0 cultures were maintained for 10 to 14 days with feeding at every 3 days following establishment of cultures at about 7 days. At 80% to 90% confluence, the Pass 0 cultures were sub-cultured using TRYPLE™ Select (1×) (Gibco, Catalog Number 12563-029) using 30-minute incubation at 37° C. with agitation at 75 RPM. Similar procedures were used in Passes 1, 2, and 3, except that T-1000 flasks were used for these passages and appropriate scale-up in media volumes used for culture, TRYPLE™ Select, etc.

### Results

A single T-75 culture of pass 0 cells yielded on average 8 to 10 million MSCs at >85% viability. Subsequent passages yielded 10 to 12-fold increases in the number of inoculated MSCs at greater than 90% viability.

The genotype was found to be:

TABLE 5
Genotype Results

| | | | | | |
|---|---|---|---|---|---|
| Amelogenin | X | Y | D18S51 | 16 | 17 |
| vWA | 17 | 18 | Penta E | 14 | 20 |
| D8S1179 | 14 | | D5S818 | 13 | |
| TPOX | 8 | 9 | D13S317 | 9 | 13 |
| FGA | 19 | 24 | D7S820 | 10 | 12 |
| D3S1358 | 16 | 17 | D16S539 | 10 | 13 |
| THO1 | 7 | 9 | CSF1PO | 10 | 11 |
| D21S11 | 30 | 31.2 | Penta D | 11 | 12 |

### Example 11: Long COVID Patients Treated with Umbilical Cord-Derived MSCs

Patients diagnosed with long COVID by persistent adverse symptoms greater than four weeks after testing positive for COVID-19 by validated RT-PCR measurements were treated with umbilical cord MSCs as described in Example 10. The deployment was by IV infusion in normal saline solution of 100 million cells over 20 to 30 minutes. No SAEs were reported nor were there any adverse events or side-effects from the treatment. During post-treatment periods these patients were also administered oral formations containing curcumin and quercetin at dosages to maintain serum levels of both compounds at or near 500 nM.

Table 6 shows patient reported outcomes from patient ID number 001J. Symptoms are listed in the left column. The column labeled Pre-is the patient reported outcome of the severity 5 of symptom on a 0-5 scale with 5 being the most intense and 0 being the least. The column labeled “Post-” is the patient-reported outcome after the response time in days after the IV infusion of MSCs. Patient 001J reported that the noted symptom remission persisted for at least 6-9 months. The comments were provided by the patient. Similar results were reported in 3 other patients. Also, lab results showed remission of anaphylaxis and Lupus symptoms in one of the four patients that were treated with UC-MSCs. Similarly, Table 7 shows patient reported outcomes from patient ID number 003T.

TABLE 6
Pre- and Post-treatment Symptoms of Long COVID in ALLORX ® STEM CELL Trial
Pre- and Post-treatment Symptoms of Long COVID in ALLORX ® STEM CELL Trial

| | | | Response | |
|---|---|---|---|---|
| Symptom | Pre- | Post- | Time (days) | Comments |
| Tiredness or fatigue | 4 | 1 | 5 | Major factor resulting in less capacity to |
| | | | | work for long periods of time without tiring |
| Post exertion issues | 4 | 2 | 5 | Exertion increased heart rate and out of |
| | | | | breathe; improved with treatment |
| Brain fog | 4 | 1 | 3 | Prior to treatment, could not even find my |
| | | | | doctor's office; clarity of presence and |
| | | | | thinking with treatment |
| Cough | 3 | 1 | 3 | Lungs cleared up |
| Chest or stomach pain | 1 | 1 | No change | Residual soreness of throat area came |
| | | | | back after a year |
| Headache | 0 | 0 | N/A | Did not experience |
| Tachycardia | 0 | 0 | N/A | Did not experience |
| Joint or muscle pain | 2 | 1 | 5 | Muscle pain reduced after treatment |
| “Pins-and-needle | 3 | 1 | 10 | Numbness pins and needle in right hand |
| | | | | reduced after treatment |
| Diarrhea | 4 | 2 | 5 | Significant reduction after treatment; |
| | | | | intermittent remains post COVID |
| Sleep problems | 1 | 0 | 3 | Back to full night's sleep |
| Fever | 0 | 0 | N/A | Never had a fever even though tested |
| | | | | positive twice |
| Dizziness when | 3 | 0 | 3 | Lightheadedness upon standing; resolved |
| standing | | | | with treatment |
| Rash | 5 | 1 | 30 | Broke out with rashes on both arms; ne |
| | | | | year to 90% improvement; comes and |
| | | | | goes |
| Mood changes | 2 | 0 | 3 | Surroundings felt like 2D; treatment |
| | | | | brought back 3D and vivid colors; normal |
| | | | | after a year |
| Smell or taste reduction | 1 | 0 | 3 | Never had a good sense of smell; slightly |
| | | | | improved; taste came back to normal |
| Menstrual changes | 0 | 0 | N/A | I'm not a woman |

* Please rate symptoms on a 0 to 5 scale (0 being least and 5 being most prevalent)

TABLE 7
Pre- and Post-treatment Symptoms of Long COVID in ALLORX STEM CELL ® Trial
Pre- and Post-treatment Symptoms of Long COVID in ALLORX STEM CELL ® Trial

| | | | | Days after |
|---|---|---|---|---|
| Symptom | ID# | Pre-treatment* | Post-treatment* | treatment |
| — | 003T | — | — | — |
| Tiredness or fatigue | — | 5 | 1 | 14 |
| Post exertion issues | — | 4 | 2 | 14 |
| Brain fog | — | 5 | 2 | 28 |
| Cough | — | 5 | 1 | 7 |
| Chest or stomach pain | — | 4 | 1 | 7 |
| Headache | — | NA | — | — |
| Tachycardia | — | 3 | 1 | 7 to 14 |
| Joint or muscle pain | — | | — | — |
| “Pin-and-needles” | — | NA | — | — |
| Diarrhea | — | NA | — | — |
| Sleep problems | — | Apnea; O2 sat | 2 | 7 |
| | | was 70% 5 | | |
| Fever | — | NA | — | — |
| Dizziness when standing | — | NA | — | — |
| Rash | — | NA | — | — |
| Mood Changes | — | 3 | 1 | 14 |
| Smell or taste reduction | — | NA | — | — |
| Menstrual changes | — | NA | — | — |

*Please rate symptoms on a 0 to 5 scale (0 being least and 5 being most prevalent)

| | | | | |
|---|---|---|---|---|
| Lymph nodes | — | 4 | 2 | Continues to go |
| | | | | down |

PATIENT COMMENTS:
General: At pre-treatment, it felt like breathing through a straw. I wasn't able to breathe deep; cough was continuous
Treated in September, remission lasted about 6 months
“Wish I had done it sooner. Infected with COVID in November of 2020 and went through 11 months of anguish.”
Wants to be in the IND authorized clinical trial.
Clinical data pre-treatment could be followed with clinical trial data.

Since other modifications and changes varied to fit particular operating requirements and environments will be apparent to those skilled in the art, this disclosure is not considered limited to the example chosen for purposes of this disclosure and covers all changes and modifications which does not constitute departures from the true spirit and scope of this disclosure.

### Example 12 Treatment of Acute Respiratory Distress Secondary to COVID-19 by IV Infusion of Umbilical Cord-Derived MSCs, ALLOWRX STEM CELLS®

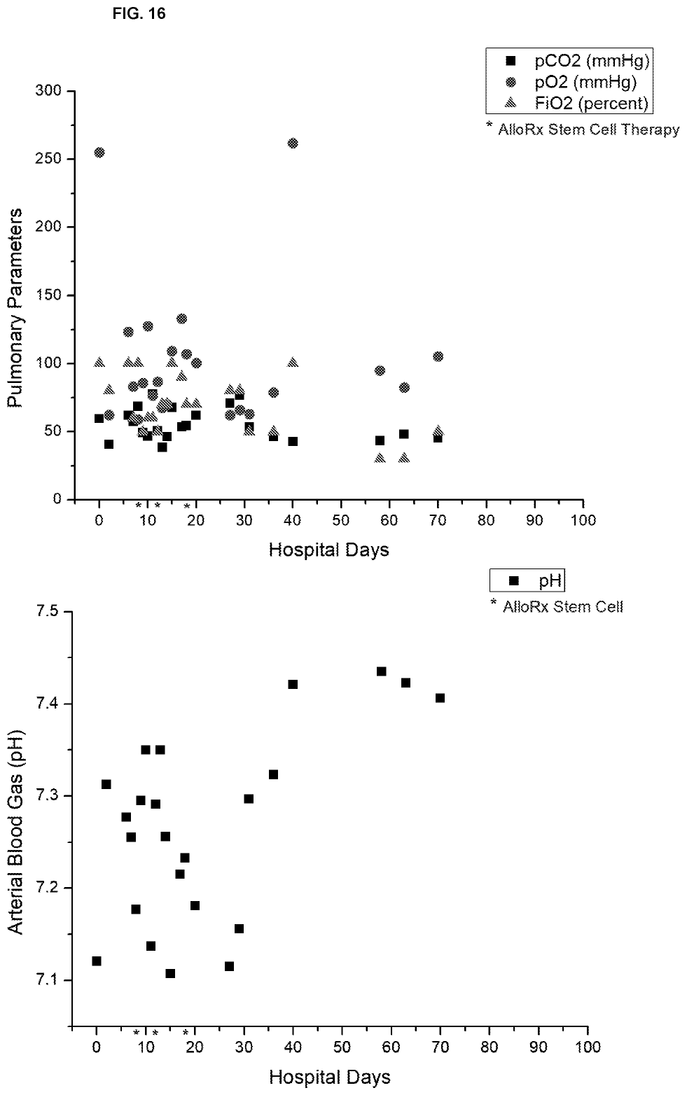

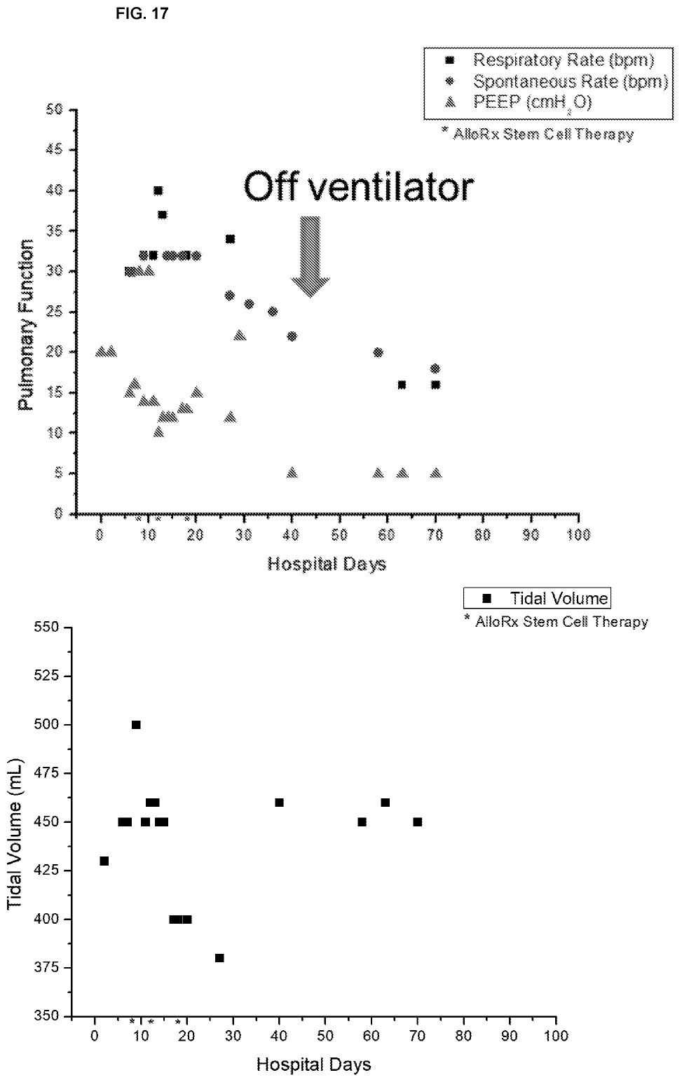

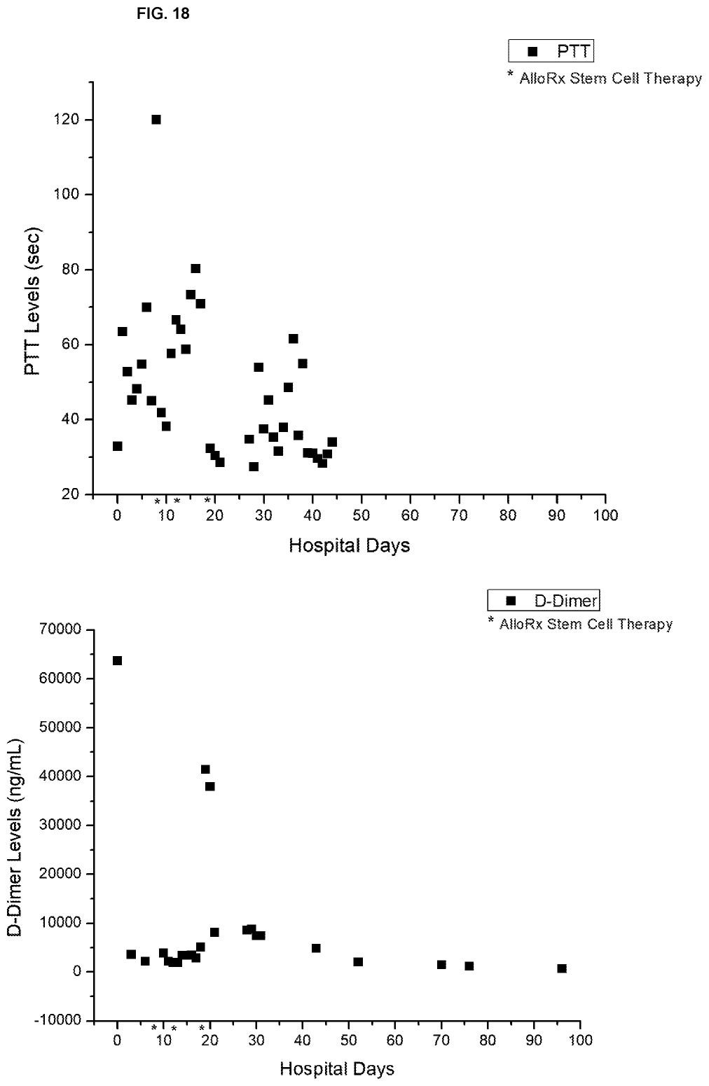

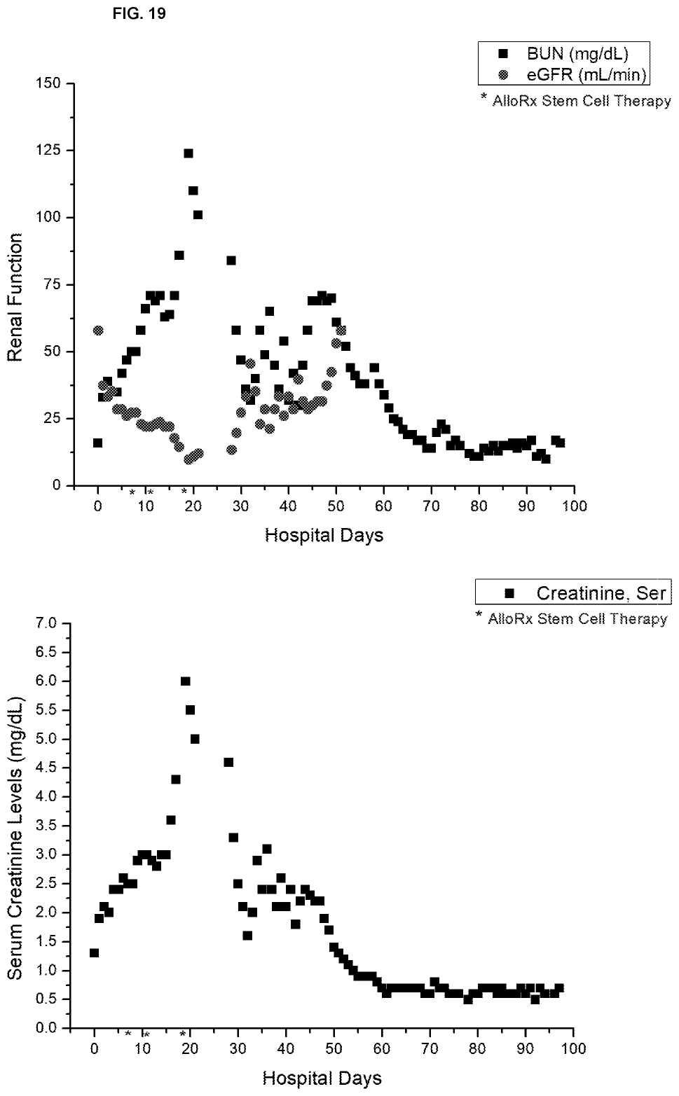

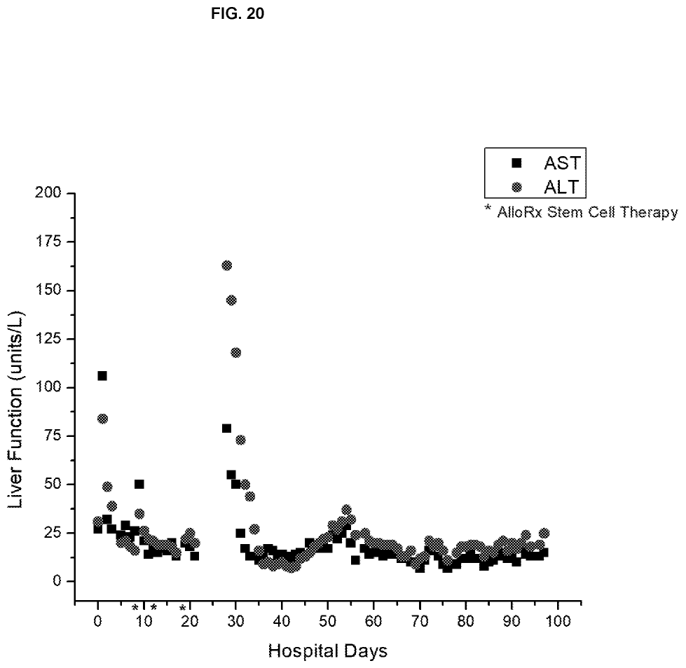

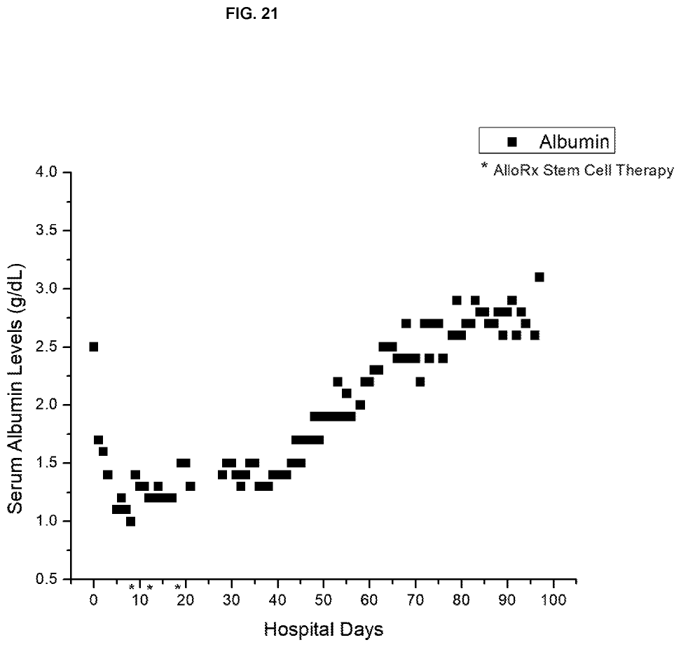

FIG. 16 to FIG. 21 show various clinical parameters of a COVID-19 patient before during and after treatment with a total of 300 million ALLORX STEM CELLS® over a time period of 100 days. The attending physician report of this patient is reproduced here: A 52-year-old male with past medical history of diabetes, hypertension, and previous history of CVA with no residual deficits presented initially for evaluation of worsening shortness of breath of two-week duration. Upon arrival, patient was found to be in acute respiratory failure with RR 30, BP 223/161, HR 152, saturating 76% on room air and was subsequently intubated. Physical examination revealed patient to be tachycardic, diaphoretic, and in severe respiratory distress with associated rales. Laboratory studies including the metabolic panel were significant for sodium 129, potassium 3.2, Chloride 92, blood glucose 454, anion gap 24 with bicarbonate 13, and beta-hydroxybutyrate levels greater than 4.50. Initial blood urea nitrogen (BUN) and creatinine levels were found to be 16 and 1.3, respectively. Initial aspartate transaminase (AST), alanine aminotransferase (ALT) and Alkaline Phosphatase were found to be 27, 31 and 157, respectively. Hematologic workup showed patient with a white blood cell (WBC) count of 23800 with 90% neutrophilic predominance and an absolute lymphocyte count of 800. Hemoglobin (14.7) and Platelet counts (353000) were unremarkable. Patient was also found to have a Lactic acid 4.2 and a Procalcitonin 0.59. Inflammatory workup revealed LDH 753, Ferritin 849, CRP 17, and an elevated d-dimer 63735. Initial arterial blood gas (ABG) revealed a pH 7.121, pCO2 60, pO2 255 on 100% FiO₂ (PF ratio 255) and PEEP 12. Chest radiograph revealed extensive patchy bilateral infiltrates, compatible with multifocal pneumonia.

Upon initial evaluation, patient was admitted to MICU with a presumed diagnosis of SARS-CoV-2 pneumonia leading to ventilator dependent respiratory failure. The SARS-CoV-2 by NAA test returned positive over the next 24 hours. Patient was also treated for diabetic ketoacidosis with an insulin drip initially and was given a 2-liter normal saline bolus. Besides, he was also immediately started on therapeutic anticoagulation with heparin drip, as well as empiric antibiotic coverage with Ceftriaxone and Doxycycline per infectious disease recommendations. Once the coronavirus test had resulted, patient underwent convalescent plasma transfusion. Despite this, over the next few days, his ventilatory status continued to deteriorate. By Day 6, his creatinine had worsened to 2.6 and he was also requiring increased ventilatory support. His arterial blood gas at the time showed pH 7.115, pCO2 85, pO2 79 on FiO2 80% (PF ratio 99), PEEP 18, TV 450. Interventions such as a trial of paralytic and prone positioning were carried out at this time besides matching adequate ventilatory support. Patient had also required episodic vasopressor support throughout his time after initial intubation.

By Day 6, patient was deemed to be a candidate for mesenchymal stem cell transplant (MSCT) and was administered a first dose by IV on Day 8 after receiving appropriate consents and approval for the same. On the day of receiving MSCT, the patient's ABG showed pH 7.177, pCO2 69, pO2 59 on 100% FiO2 (PF ratio 59), PEEP 14. Within the next 18 hours of receiving MSCT, the patient's respiratory status improved significantly and ABG results at the time showed pH 7.30, pCO2 49, pO2 86 on 50% FiO2 (PF ratio 172) and PEEP 14. For the initial dose, part of MSCT was administered via inhalation but it appeared to cause tongue swelling which improved with a dose of diphenhydramine. PEEP was subsequently gradually decreased to 10 maintaining adequate oxygenation. Patient demonstrated a period of improvement initially, but oxygenating requirements started to rise again on Day 12 requiring PEEP 12 and FiO2 70%. ABG at the time showed pH 7.23, pCO2 49, pO2 60 (PF ratio 86). Patient was titrated off vasopressors. MSCT was again administered by IV on Day 12 with equivocal response. By Day 15, patient began to deteriorate again requiring 100% FiO2. Requirements were slowly decreased to 80% FiO2 and PEEP 13 with a pO2 of 66 on ABG (PF ratio 83). Patient received a third dose of MSCT by IV on Day 18. At this point, patient's creatinine had also increased to 6, and patient was started on hemodialysis for anuria, worsening kidney function, and volume overload. Within the next 48-72 hours, the patient's oxygenation requirements showed dramatic improvement with ABG showing 7.26, pCO2 58, pO2 105 on FiO2 60% (PF ratio 175) and PEEP 10.

On Day 23, admission was complicated by worsening oxygenation and desaturating. Patient was found to have a white-out of his left lung that improved with bronchoscopy and suctioning out mucus plug. A lavage done at the time grew growing gram-negative rods. Patient was being treated with meropenem and levofloxacin since patient went into shock, was septic, and required vasopressor support. Patient also required a brief course of amiodarone during this time and experienced episodes of atrial fibrillation with rapid ventricular rate. This was since titrated off. By Day 34, patient had a repeat coronavirus test done that came back negative. Patient had been somnolent without sedation for about two weeks at this point. During this time, patient was also transitioned to a tracheostomy placement to replace endotracheal tube. Neurologic assessment revealed patient to be breathing spontaneously and reactive pupils, but not responding to verbal commands or pain. A subsequent CT scan of the head showed hypodensity involving bilateral parietal (right greater than left), right temporal and right occipital lobes and right basilar ganglia and thalamus as well. This constellation of findings was concerning for age indeterminant infarcts. We presumed patient's mental status considering extensive infarcts, and impressive CT scan findings was minimal.

On Day 39, after another repeat coronavirus testing was negative, patient had a PEG tube placement. Patient was transitioned to a permanent hemodialysis catheter. Oxygenation had improved significantly at this point with ABG showing 7.42/43/105 on 40% FiO₂ (PF ratio 263) and PEEP 5. By this point, a review of comprehensive labs suggested a peak creatinine 6 (Day 25), d-dimer 63735 (Day 0), and CRP of 27.7 (Day 30). AST and ALT peaked at 232 (Day 27) and 203 (Day 27).

By Day 48, renal function started to recover, and patient started to make urine. Patient was taken off hemodialysis and his kidney function improved dramatically over the next few days. By Day 49, patient began to demonstrate improvement in mental status with opening and closing of the eyes and trying to orient to commands. By Day 52, patient had a dramatic improvement in neurologic status with intact pupillary, gag, and corneal reflexes. Patient also demonstrated following commands and moving extremities on right. Patient probably had a left sided hemiparesis at this time as evidenced from lack of movement from the left side. By Day 60, patient had been off sedation, following commands, keeping eyes open spontaneously open, and able to whisper with tracheostomy in place. Patient also started tolerating tracheostomy collar for majority of the day still requiring ventilatory support at night. The care team continued to work tirelessly to transition further care with physical therapy and speech therapy. Patient also started tolerating feeds. A repeat CT Head done at this point showed right PCA distribution subacute infarct along with moderate regional mass effect. A repeat CT chest demonstrated interval clearing of previously seen patchy pulmonary opacities with pattern suggesting resolving pneumonia. Resolution of previous right pleural effusion was also seen. Presently, D-dimer, CRP and Creatinine are 2078, 0.8, and 0.7 respectively with excellent kidney function and adequate urinary output. AST and ALT are 15 and 20, respectively. Most recent ABG done on Day 59 showed pH 7.435, pCO2 43.3 and pO2 94.8 on 30% FiO2 (PF ratio 316), PEEP 5. These values currently assure us of how far the patient has come since the original grim prognosis.

### Example 13: Manufacturing Methods for Production of Umbilical-Cord Derived MSCs and Exosomes

### 2D Cell Culture in Planar Monolayers:

All reagents used in processing and manufacturing are sterile filtered into autoclave (121° C. for 50 minutes) or gamma-irradiated (sterile) containers and transferred into an International Standards Organization (ISO) 7 clean room. When all materials are transferred into the ISO 7 cleanroom, sterile 70% isopropyl alcohol, sterile 3% hydrogen peroxide, and a sporocide are used to for sanitizing materials. All processing and manufacturing are done under an ISO 5 biological safety cabinet within the ISO 7 cleanroom.

The umbilical cords are collected at birth into a sterile bag and double bagged using aseptic techniques. The donor of the umbilical cord is selected through an American Association of Tissue Banks (AATB) accredited facility and serology and virology is performed prior to cord collection. The cord is delivered to Vitro Biopharma same day and quarantined until all donor testing is completed and reported. Once released, the umbilical cord is brought into an ISO 7 cleanroom. The bag containing the cord is cleaned and sanitized. It is then transferred into a sterile ISO 5 biological safety cabinet. Once processing is complete, it is plated into sterile TC-coated T-75 flasks and placed in a humidified, ISO 5 tri-gas copper incubator (5% O2/5% CO2) and expanded in cell culture medium optimized for MSC growth (Vitro Biopharma, Catalog Number SC00B2 and/or SC00B1-SF) containing 1×penicillin/streptomycin and 25 μg/mL fungin.

Flasks are monitored for microbial growth over the isolation/purification period of 10-14 days. During the isolation/purification period, cells are washed with phosphate buffered saline (PBS) and fed with growth medium until confluency is greater than 90% in an ISO 5 biological safety cabinet. Cells are then sub-cultured using Accutase (Innovation Cell Technologies, Catalog number AT104) according to the manufacture's procedure. Cells are expanded to pass 1 for the creation of a Master Cell Bank and proceeds additional quality control testing. Additional cells are passed for expansion in TC-coated T-1000 flasks (Millipore-Sigma, Catalog Number PFHYS1008) for about 7-10 days for each pass. Sub-culture using Accutase is performed when cells reach 90% or more confluency. A Working Cell Bank is created from low passage (pass 2) cells to support additional future expansions. The Working Cell Bank is cryogenically preserved in Vitro Biopharma's cryopreservation media (CPM) and stored in liquid nitrogen (−196° C.). Additional quality control testing is performed on the lot of Working Cell Bank.

For additional expansions and finished cellular medicine, a vial from Working Cell Bank will be obtained and thawed in a 37° C. water bath with gentle agitation. The vials are wiped down with the three disinfectants and the label is collected and stored with the batch record. The vial is transferred into the ISO 5 biological safety cabinet and plated according to the standard operating instructions and placed in the ISO 5 tri-gas copper incubator (5% O2/5% CO2). Expansion takes 7-10 days with a wash and feed every 3 days. Microbial monitoring is provided at each expansion period. When bulk cellular dose is created, cells are sub-cultured, counted with viability and cryopreserved in drug master file cryopreservation medium (PRIME-XV Stem FreeziS DMSO-Free) and aliquoted into 5 mL leak-proof cryovials (12.5M cells/mL). Each vial contains 50 million cells at 4 mLs each. All cryopreserving and packaging are performed in the ISO 5 biological safety cabinet. The vials are transferred out of the clean room into a controlled rate freezer for the freezing period (−1° C./min). Once cells reach −80° C., the vials incubate for 15 minutes at −80° C. and are transferred into liquid nitrogen (−196° C.) for quarantine storage prior to QC testing and release.

### 3D Cultures on Microcarriers in Stirred Tank Bioreactors:

There are potential advantages of 3D cultures compared to 2D including: a) Microcarrier biochemical and biophysical properties, including higher surface area/volume ratios and culture conditions can be optimized for high density cultures, b) Stirred tank bioreactors and associated bioprocessing steps are automated in closed, sterile systems readily available commercially that allow consistent control of the culture environment, c) Process probes allow online, real time monitoring of crucial cell culture conditions including cell density allowing immediate corrections if needed, d) These systems are scalable from laboratory, pilot scale to production levels with few scale-up obstacles, e) Potential cell damage due to mechanical forces such as shear can be obviated, f) The cost of production can be reduced compared to planar culture systems without sacrifice in product quality. (M. May, Microcarrier-based bioreactors can make more stem cells, GEN Feb. 22, 2022).

We performed initial small scale experiments to optimize microcarrier type and basic culture conditions in shaker flasks. We optimized binding and single passage expansion by using binding conditions resulting 2-3 cells per microcarrier and low serum levels initially (0.05%) in MSC-Gro (Vitro Biopharma, Inc. Catalog Number SC00B4) that were increased to 5% at 4-6 hours post-inoculation. The microcarrier concentration was 7,935 microcarriers per mL in the SC00B4 culture medium and the cultures were maintained in 5% O2/5% CO2 gas phase in an humidified cell culture incubator with continuous agitation at 55 RPM resulting in just suspended microcarriers. Cultures were inoculated with 0.952×106 cells in 40 ml SC00B4. Table 8 shows the cell counts and viability.

TABLE 8
Cell Counts and Viability at 7 Days Post-Inoculation

| | | | | | | | | Fold |
|---|---|---|---|---|---|---|---|---|
| | | | | | | | | increase |
| | | | | | | | | from |
| | Count 1 | Count 2 | Count 3 | Avg | [x] | Total | StdDev | inoculation |
| Fact III | | | | | | | | |
| Cells | 4.15E+05 | 2.58E+05 | 1.85E+05 | 2.86E+05 | 8.58E+05 | 3.43E+07 | 117528.7 | 36.00 |
| Viability | 98.00% | 97.40% | 95.10% | 96.83% | 96.80% | | 0.015308 | |
| Collagen | | | | | | | | |
| Cells | 4.90E+05 | 4.00E+05 | 3.41E+05 | 4.10E+05 | 1.23E+06 | 4.92E+07 | 75035.55 | 51.7 |
| Viability | 96.10% | 93.80% | 95.30% | 95.07% | 95.00% | | 0.011676 | |

These results show an average 44-fold increase in cell count during 7 days culture within the shaker flasks and the MSC cell viability was greater than 95%. This is a considerable increase over the similar expansion in planar, 2D flasks that is typically 10-fold or less.

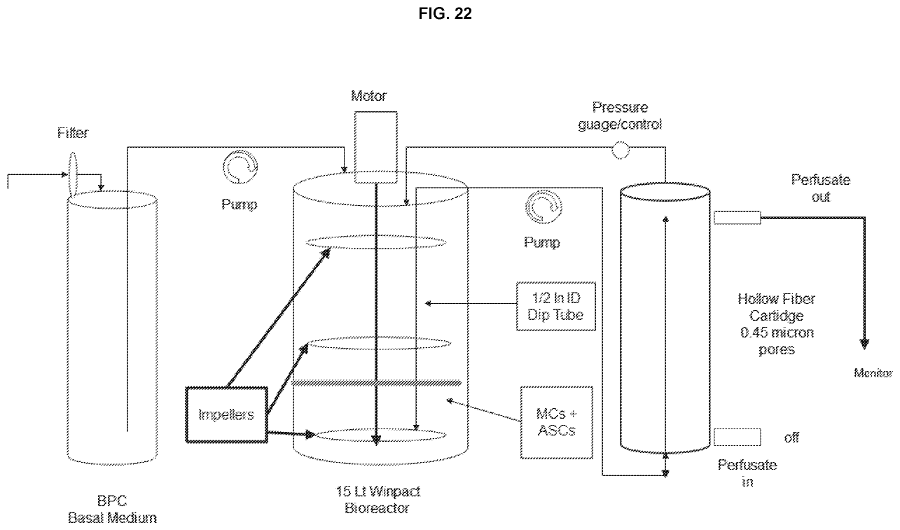

Also, FIGS. 22 & 23 show diagrams of the procedure to collect ALLOEX EXOSOMES® from stirred tank bioreactors using the inoculation conditions described above. Diafiltration as shown in FIG. 22, is automated dialysis that is used to exchange the growth medium with basal medium needed to produce the initial MSC-derived product, exosome-containing conditioned medium (ALLOEX EXOSOMES®). This product has commercial applications in cosmetics and development of regenerative medicine biologics derived from exosomes and other products secreted from cultured MSCs.

Initially, the impellers of the bioreactor that maintain a homogeneous mixture of MSCs (ALLORX STEM CELLS®) attached to microcarriers in growth medium are turned off allowing the MC/ASC complex to settle to the bottom of the bioreactor as illustrated below the blue line. This happens at the transition of the growth curve from exponential to the plateau phase as determined by a capacitance monitoring probe, about 8 to 10 days following inoculation and continuous culture in the bioreactor. This mixture is then diluted % or less in basal medium to reduce viscosity and pumped out of the bioreactor and through the 0.45 micron hollow fiber cartridge without applied back pressure and back into the bioreactor. Back pressure is then applied to the HF cartridge outlet to create cross-flow filtration and create perfusate outward flow. The flow rate from the basal medium BPC into the bioreactor is set to equal the perfusate outflow rate from the HF cartridge. The process continues until 5 to 6 volumes of basal medium has been pumped through the system, which is sufficient to exchange the growth with basal medium in the culture consisting of UC-MSCs attached to microcarriers. Critical process parameters include fluid flow dynamics (flow rates, tubing materials & dimensions) to prevent fouling, use of diaphragm pumps for MC/stem cell complexes to optimize stem cell viability and sterility in all connections/tubing and BPCs.

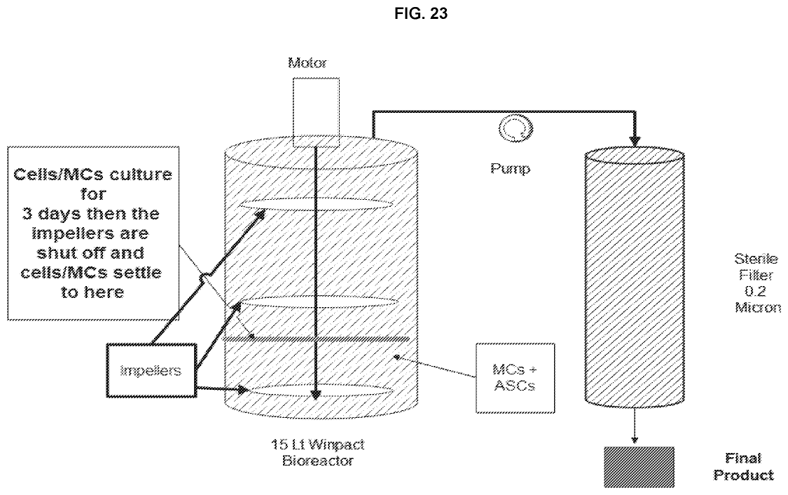

Following diafiltration, the bioreactor is then filled with basal media and continuous cell culture proceeds for about three days that allows for the secretion of various growth factors and other paracrine products of MSCs, i.e., the secretome together with MSC-derived exosomes. FIG. 23 shows the process of harvest of the conditioned medium. The impellers are shut off allowing the Stem cell-microcarrier complexes to settle and separate from the conditioned medium that is then pumped out and through a sterilizing 0.2 micron filter cartridge into an appropriate Bioprocessing container as final product.

### Example 14: Quality Control and QC Release of ALLORX STEM CELLS®

Final product QC involves culture of a cell sample taken from the final product and plated on a Tryptic Soy Agar (TSA) Plate (Hardy Biologics, catalog number P34), Sabouraud Dextrose Agar (SDA) Plate (Hardy Diangostic, catalog number P36) and a Brucella Blood Agar (BBA) Plate (Anaerobe Systems, catalog number AS-141) followed by incubation at 37° C. for 72 hours. Unites States Pharmacopeia (USP)<71> sterility testing is performed followed by a 14 day incubation period. Negative results on plates and USP<71> are QC release criterion.

USP<63> mycoplasma testing is performed. The absence of mycoplasma is determined through validated PCR assay system (eMyco Plus Mycoplasma PCR kit, Intron, catalog number 25234). This system is validated to detect over 200 known species of mycoplasma. A negative result is required to pass QC release.

Absence of bacteria is determined through PCR of 16S ribosomal RNA (Fast MicroSeq 500; Applied Biosystems). This system is validated to detect over 2000 species of bacteria. A negative result is required to pass QC release.

Absence of fungi is determined through PCR of 18S ribosomal RNA (MicroSeq D2 LSU, Applied Biosystems). This system is validated to detect over 1100 species of fungi. A negative result is required to pass QC release.

USP<85> Endotoxin Limulus Amebocyte Lysate (LAL) method is performed having an Endotoxin levels less than 0.25 EU/ml by a LAL method (ThermoFisher, catalog number A39552). The procedures described for Chromogenic LAL endotoxin conforms with those described in the FDA Guidelines. This threshold level is based on recommended levels by the FDA based on lymphatic and cardiovascular exposure levels (Guidance for Industry: Pyrogen and Endotoxin Testing; DHHS, FDA, June 2012).

Since this product may be used for intravenous drip injection into patients undergoing clinical trials, we have set our criterion for acceptance at 5-fold less than industry standard as additional assurance of sterility/absence of contamination. In process safety QC involves monitoring of microbes and sterility testing (TSA, SDA, BBA, TSB, FTM, 16S PCR, 18S PCR, Mycoplasma PCR, Chromogenic LAL Endotoxin).

### USP-71 Sterility Testing Uses:

1. In Tryptic Soy Broth (TSB), inoculate 500 μL of cells and positive controls (Staphylococcus aureus [ATCC 6538], Pseudomonas aeruginosa [ATCC 9027], Bacillus subtilis [ATCC 6633], Candida albicans [ATCC 10231], Aspergillus brasiliensis [ATCC 16404]) into a 5 mL TSB tube and place in rack at room temperature in cabinet. Incubate for 14 days at 20-25 C. Check at Day 1, 3, 5, 7, 14. Have a negative control. Obtain pictures.

2. In Fluid Thioglycollate Broth (FTB), inoculate 500 μL of cells and positive controls (Staphylococcus aureus [ATCC 6538], Pseudomonas aeruginosa [ATCC 9027]) into a 5 mL FTB tube and place in rack at room temperature in cabinet. Incubate for 14 days at 35-37° C. Check at Day 1, 3, 5, 7, 14. Have a negative control. Obtain pictures.

Human viral pathogen testing occurred by DNA sequence analysis and quantitation of specific viral nucleic acid sequences by fluorescent probe technology according to Good Manufacturing Practice (GMP) regulations found in Title 21 C.F.R. Parts 210 & 211 by a third party CRO. The human viral pathogens tested are: Parvovirus B119, Polyomavirus BKV, EBV, HAV, HBV, HCMV, HCV, HEV, HHV-6, HHV-7, HHV-8, HIV-1, HIV-2, HPV-16, HPV-18, HTLV-1, HTLV-2, JCV and SARS-Cov-2. QC release criteria requires negative test results from each viral PCR test.

In-vitro cell-based assays for viral pathogens use Hela Cells, MCR-5 and Vero76 cells tested for cytopathic effect, Hemadsorption test and Hemagglutination test according to GMP regulations found in Title 21 C.F.R. Parts 210 & 211 by a third party CRO. QC release criteria requires negative results from final product and positive results for the positive control Bovine Parainfluenza 3 virus.

In-vivo adventitious virus assay for detection of in apparent viruses in biological samples is determined according to Guidance for Industry, February 2010, Characterization and Qualification of Cell Substrates and Other Biological Materials Used in the Production of Viral Vaccines for Infectious Disease Indications, European Pharmacopoeia 2.6.16, Tests for Extraneous Agents in Viral Vaccines for Human Use Points to Consider in the Characterization of Cell Lines Used to Produce Biologicals as recommended by the US FDA Center for Biologics Evaluation and Research (1993) International Conference on Harmonization, Guidance for Industry Q5A (R1): Viral Safety Evaluation of Biotechnology Products Derived from Cell Lines of Human or Animal Origin (1999), US and EU regulations was performed in guinea pigs, post-wean and suckling mice and the embryonic chick embryo by a third party CRO.

All animals and eggs assigned to this protocol were obtained from the CRO production facilities on which routine health monitoring was performed. The Test Article was received from the Client and was inoculated via multiple routes into guinea pigs (Hartley, 350-450 grams), mice (PWM, CD-1, 15-20 grams, and suckling <24 hours) and embryonated chicken eggs (10-11 days for allantoic fluid inoculation and 6-7 days for yolk sac inoculation); the hosts were monitored. After the completion of the prescribed observation period, survival percentages were determined. Guinea pigs were submitted for gross necropsy. Appropriate specimens from the suckling mice and embryonated eggs were processed, and hemagglutination testing was performed on allantoic and yolk sac fluids. Additionally, homogenates or pools from primary inoculation groups of suckling mice and embryonated chicken eggs were passaged into secondary inoculation groups of mice and eggs. The secondary inoculation groups were monitored. The survival percentage of each secondary inoculation group was determined at the completion of the observation period and hemagglutination testing was performed on the designated specimens.

QC release criteria requires negative results from final product and positive results for all positive controls. Any unexpected results were repeated and determined to be of non-cellular origin.

Purity and identity were determined by testing to International Society for Cell and Gene Therapy (ISCT) standards for MSC definition:
Adherence to plastic, phenotype consisting of: CD11b−; CD14−; CD19−; CD34−; CD44+; CD45−; CD73+; CD79a−; CD90+, CD105+; CD126−; HLA-DR− by flow cytometry.
Demonstrated tri-lineage differentiation (e.g., differentiation into bone, cartilage, and fat cell). Human karyotype and human DNA finger print test.
Purity >95% by flow cytometry.
Potency to QC release criteria by cellular ATP content and gamma-interferon induced IDO activity.

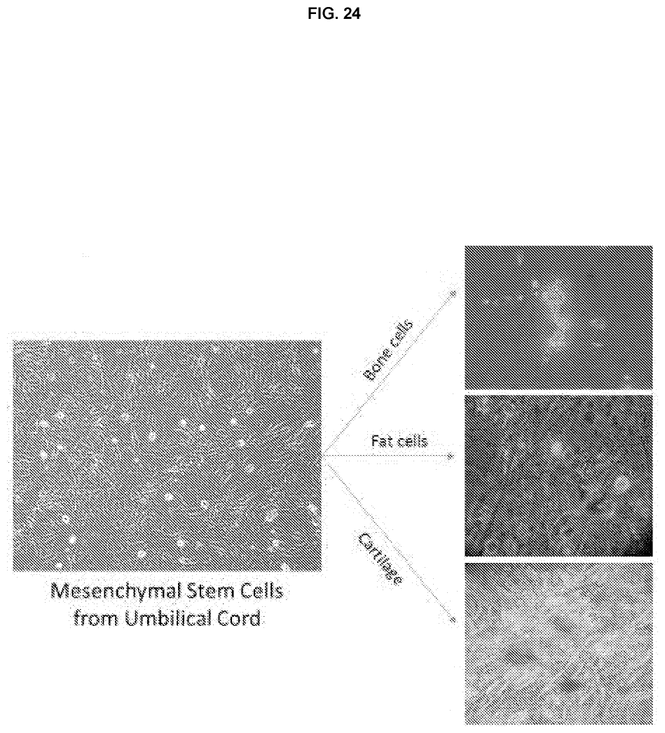

QC release criteria include required identity by phenotypic marker flow cytometry analysis, trilineage differentiation, human karyotype and human DNA test results, purity >95% and potency by criterion levels of ATP cellular content and IDO levels. See FIG. 24.

### Example 15: Exosome-Containing Conditioned Medium (AlloEx Exosome) Analysis

Exosomes within ALLOEX EXOSOMES® were first purified by size exclusion chromatography that resolves particles by size. Larger particles elute first on the column, followed by proteins and small molecular weight compounds. An Izon 35 nm qEV SEC column was pre-equilibrated with 20 mL of freshly filtered PBS. With cap closed, the buffer from the top of the column was removed and 500 μL of the sample was loaded. Cap was opened immediately and 0.5 mL fractions were collected. The column was not allowed to dry out at any time, and fresh PBS was added at the top when needed to maintain the flow. First 6 fractions (3 mL)-void volume, were discarded. Exosome fractions 7, 8 and 9 were collected and pooled together. The exosome fractions were concentrated using Amicon Ultra 0.5 30 kDa MWCO centrifugal filter devices. A total of 50 μL was recovered after centrifugation and transferred into a new tube. The filter membranes were rinsed with 100 μL of PBS by pipetting up and down 10 times. The wash buffer was combined with the retained exosomes and total of about 150 μL of exosomes in PBS was collected for further analysis.

Fluorescent NTA technique involves labeling of intact exosomal membrane with a fluorescent dye and then performing the analysis in scatter and fluorescent modes. This technique allows exclusion of contaminant particles, such as protein aggregates, lipoproteins, etc from analysis and assessment of the purity of exosome sample. The analysis was performed with Zetaview (Particle Metrix) instrument equipped with 520 nm laser, 550 nm long pass cut off filter and sCMOS camera. DI water was filtered on the day of analysis through 0.22 μm syringe filter and its purity confirmed by NTA prior to the study. Exosome labeling was done using Exoglow fluorescent NTA labeling kit from System Biosciences according to manufacturer's protocol. Briefly, 12 μL of reaction buffer were mixed with 2 μL of dye and 36 μL of sample. The mixture was vortexed for 15 seconds to mix well and samples were incubated at RT for 10 minutes. Liposomes (provided with kit) were used as labeling control: 1 μL of liposomes was mixed with 12 μL of reaction buffer and 2 μL of dye. Dilutions were made by mixing DI water filtered through 0.2 μm syringe filter with corresponding volume of a sample.

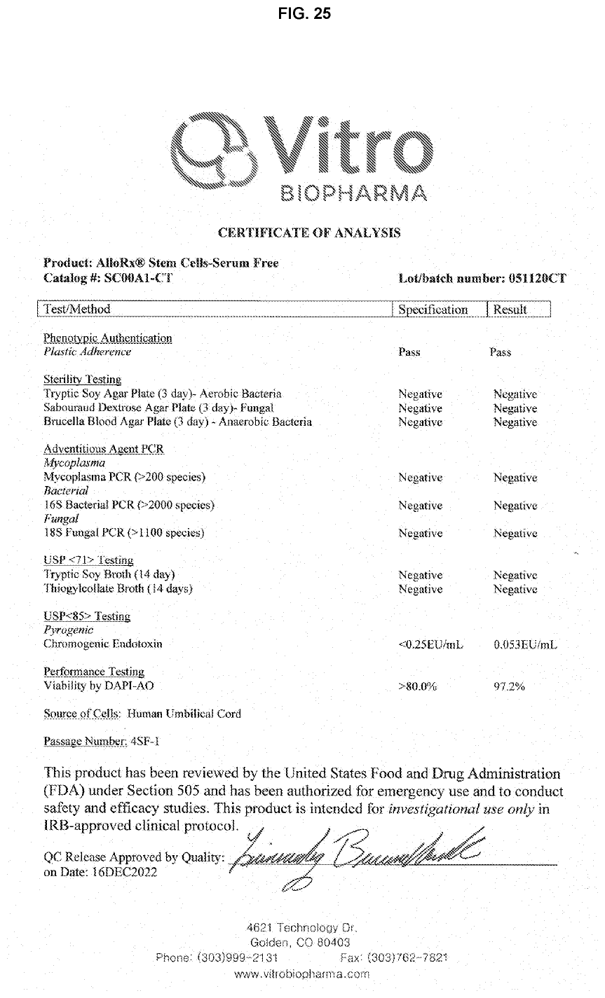

The results are shown in FIG. 25. Lot 021422 ALLOEX EXOSOMES® (Exosome-containing Conditioned medium) contained 69 billion exosomes/ml that averaged 165 nm in diameter at 94.2% purity and were positive for exosome biomarkers: CD9 (21.7%), CD63 (21.7%) and CD81 (6%), indicating identity as exosomes, although strict standards of exosome identity, purity and potency have not yet been established.

### Example 16: Clinical Trial Results Using ALLORX STEM CELLS®

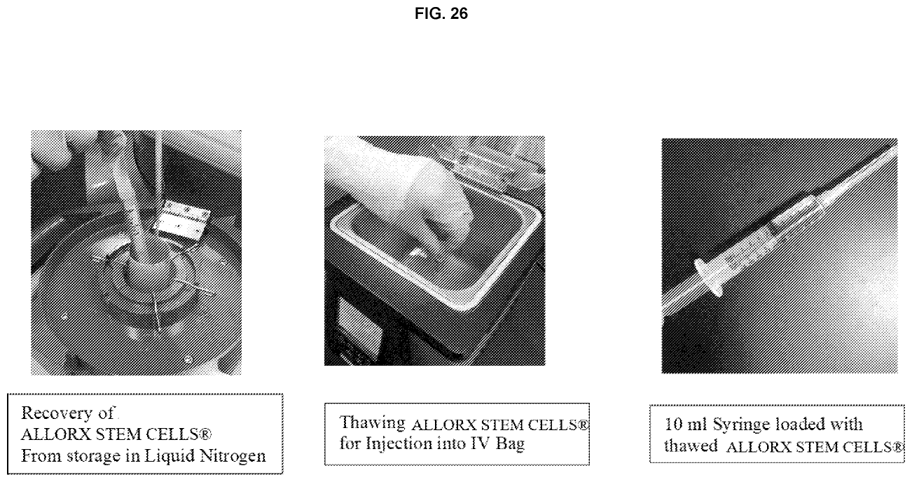

Table 9 below shows the results of various clinical trials performed using ALLORX STEM CELLS® manufactured and subjected to quality control procedures as described above. These studies were overseen by IRBs using approved protocols and administration of varying dosages of ALLORX STEM CELLS®. FIG. 26 describes the process of preparation of the cells for IV infusion or direct injection.

TABLE 9
Results of Various Clinical Trials

| | | | | Dosing | |
|---|---|---|---|---|---|
| | | | Dose/No. of | Regimen/ | |
| Mode of | Study | # of | Cells | Duration of | |
| Admin. | Population | Subjects | (million) | treatment | Summary of Safety |
| i.v. | AD | 3 | 300m per | 1 (once) | No SAE's or AE's (self- |
| | | | treatment | | resolving AE's if any) |
| i.v. | ALS | 14 | 100m per | 1-3 times | No SAE's or AE's (self- |
| | | | treatment | | resolving AE's if any) |
| i.v. | Autism | 3 | 100m - 300m | 1 (once) | No SAE's or AE's (self- |
| | | | per treatment | | resolving AE's if any) |
| i.v. | Crohn's | 5 | 300m per | 1 (once) | No SAE's or AE's (self- |
| | Disease | | treatment | | resolving AE's if any) |
| i.v. | Chronic | 3 | 300m per | 1 (once) | No SAE's or AE's (self- |
| | Kidney | | treatment | | resolving AE's if any) |
| | Disease | | | | |
| i.v. | COPD | 7 | 300m per | 1 (once) | No SAE's or AE's (self- |
| | | | treatment | | resolving AE's if any) |
| i.v. | Diabetes | 6 | 100m per | 1 (once) | No SAE's or AE's (self- |
| | | | treatment | | resolving AE's if any) |
| i.v. | Long COVID I | 33 | 150m per | 1 (once) | No SAE's or AE's (self- |
| | COVID | | treatment | | resolving AE's if any) |
| i.v. | Lupus \| Lyme I | 119 | 150m or | 1-2 times | No SAE's or AE's (self- |
| | RAI | | 300m/per | | resolving AE's if any) |
| | Fibromyalgia | | treatment | | |
| | Inflammatory | | | | |
| i.v. | Multiple | 43 | 300m per | 1 (once) | No SAE's or AE's (self- |
| | Sclerosis | | treatment | | resolving AE's if any) |
| i.t. or | Osteoarthritis | 30 | 100m to | 1-3 times | No SAE's or AE's (self- |
| i.m. | | | 300m | | resolving AE's if any) |
| | | | depend on | | |
| | | | site | | |
| i.v. | Parkinson's & | 10 | 300m per | 1 (once) | No SAE's or AE's (self- |
| | Muscular | | treatment | | resolving AE's if any) |
| | Dystrophy | | | | |
| i.v. | Other | 26 | Varies | Varies | No SAE's or AE's (self- |
| | | | | | resolving AE's if any) |

TOTAL = 301 Patients

These trials were Phase I/II open label, non-randomized, nor placebo controlled thus there are no direct comparisons with non-treated patients. The efficacy results were mainly anecdotal reports of symptom remission by patients. For example, a case study of a multiple sclerosis (MS) patient treated illustrates this efficacy. This MS patient was treated by three successive infusions of 300 million MSCs. The first treatment used autologous adipose-derived MSCs and resulted reduction of neurological symptoms for 4 months followed by relapse to pre-treatment levels of neurological function, as reported by the patient. Approximately 4 years later, this patient received two successive IV infusions of 300 million ALLORX STEM CELLS®. The initial treatment—as reported by patient—provided relief and a significant decrease in neurological symptoms (Left leg foot drop) for 18 months succeeding the treatment. Subsequently, the patient underwent another IV infusion therapy of 300 million ALLORX STEM CELLS® and reported further improvements in neurological symptoms including restoration of thermal sensory function in his left arm and a dramatic increase in energy level compared to the normal daily fatigue that is a common feature of MS. This patient received a cross-over protocol design in that the initial dosage was autologous adipose-derived MSCs while the subsequent two treatments consisted the allogeneic umbilical cord-derived MSCs (e.g., ALLORX STEM CELLS®). Since in-vitro results showed significantly increased potency of umbilical cord MSCs (ALLORX STEM CELLS®) over adipose-derived MSCs by both mitochondrial and immunosuppression, the clinical outcomes corroborate the potency measurements by cellular ATP levels and gamma IFN-induced IDO activity. Thus, significantly greater potency of UC-MSCs yields longer, more sustainable remission of MS symptoms than adipose-derived MSCs.

### Example 17: Comparative Analysis of Adult Mesenchymal Stem Cells Derived from Adipose, Bone-Marrow, Placenta, and Umbilical Cord Tissue

Mesenchymal stromal/stem cells (MSCs) have the potential to repair and regenerate damaged tissues, making them attractive candidates for cell-based therapies. Expanded and well-characterized MSCs have application in regenerative medicine and have been used in several clinical trials including treatment for osteoarthritis and other conditions. Here, we provide results of a comparative study of purified and expanded MSCs from adipose, bone-marrow, placenta, and umbilical cord involving determination of phenotype by flow cytometry analysis, cellular potency by quantitative assessment of mitochondrial function and immunosuppression, and cellular function by quantitative assessment of cell migration and proliferation. Our results show comparable phenotypic profiles, morphology, expansion in cell culture and adipogenic, osteogenic and chondrogenic differentiation. Potency measures of mitochondrial/immunosuppressive capacity and additional cellular function assays show differences suggesting biological advantages of umbilical MSCs.

Mesenchymal stem cells (MSCs) are multipotent, non-differentiated adult stem cells capable of self-renewal, proliferation, conversion into differentiated cells as well as the regeneration of tissues. MSC-based regenerative medicine offers novel therapies for patients with injuries, end-stage organ failure, degenerative diseases, and several other medical conditions. Transplanted MSCs have shown potential therapeutic benefits and safety in myocardial, musculoskeletal, neurological, autoimmune disorders, and several other disorders (Carralho E, et al. Regen. Med. 2015; 10:1025; Freitag J, et al. BMC Musculoskeletal Disorders 2016; 17: 230; Neirinckx V, et al. Stem Cell Trans. Med 2013; 2: 284; Munir H and McGettrick, HM Stem Cells Dev. 2015; 24:2091). MSCs are isolated from several tissues including lipoaspirates, perinatal tissues, cord blood, teeth, etc. and have considerable capacity for in vitro expansion and broad regenerative potential. These properties make MSCs attractive candidates for cell-based therapies.

No MSC-based therapies are yet approved for clinical application in the US while hematopoietic stem cells are FDA-approved for clinical use. The European Medicines Agency has recently approved allogeneic MSCs (Alifosel™) derived from adipose-tissue for treatment of a type of Crohn's disease. Other countries have different regulatory requirements for commercial approval of stem cell therapies.

Clinical trials based on expanded MSCs are common internationally, although there are variations in the degree of regulation including the requirements for adherence to cGMP standards. Here, we report on phenotypic and functional characterization of purified and expanded MSCs from adipose, bone-marrow, placenta, and umbilical cord. Our results show comparable growth and tri-lineage differentiation performance while umbilical cord MSCs display enhanced potency, cellular functions and capacity for differentiation into neural stem cells.

### Results

### Growth in Cell Culture

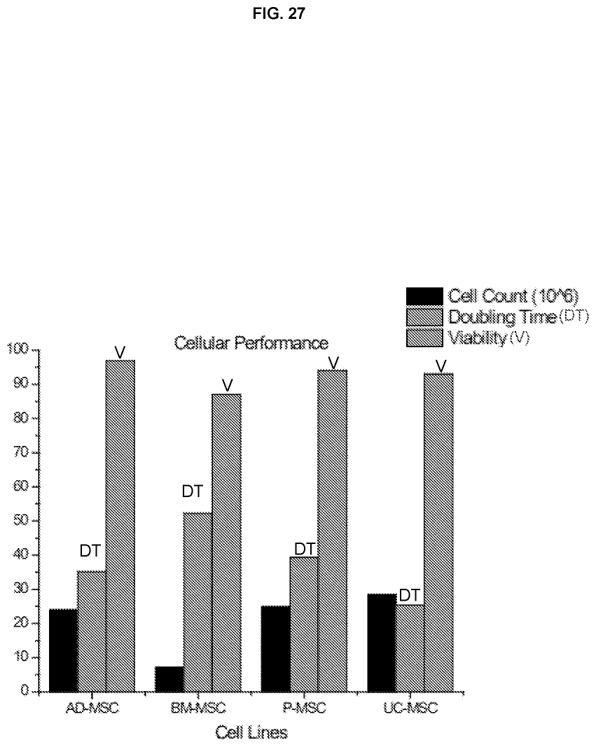

We initially compared the growth and expansion characteristics of AD-MSCs, BM-MSCs, P-MSCs and UC-MSCs following pass 2 in cell culture as described above. The results shown in FIG. 27 at pass 2 show doubling time (Td) between 20 hours for UC-MSCs and 53 hours for BM-MSCs while MSC viability was relatively consistent at 85 to 95%. This shows comparable expansion at variable growth rates: UC-MSC>AD-MSC>P-MSC>BM-MSC. Similar results were seen in several replicates (n=4) of this protocol.

### Phenotypic Characterization of AD-MSCs, BM-MSCs, P-MSCs, and UC-MSCs

The isolated and expanded AD-MSCs, BM-MSCs, P-MSCs, and UC-MSCs were investigated for MSC phenotype at P2 by staining for cell surface markers, which were detected using flow cytometry according to the ISCT standard (Dominici M, et al. Cytotherapy. 2006; 8: 315) and the results are shown in Table 10. The AD-MSCs and UC-MSCs expressed the typical MSC markers CD90, CD73, and CD105. In addition, the cells showed low expression of hematopoietic markers CD11b, CD14, CD19, CD34, CD45, and the MHC class II molecule HLA-DR. Similar results have been seen in several replicates (n=4). However, the P-MSCs expressed a high level of CD45, possibly due to leukocyte contamination. The BM-MSCs also expressed higher levels of CD45 and CD79a, possibly due to residual levels of B-cells.

TABLE 10
Summary results of flow cytometry analysis of UC-MSCs, P-MSCs, AD-
MSCs & BM-MSCs compared to the ISCT standard definition of an MSC

| Cell Surface Markers | ISCT Standard | UC-MSC | P-MSC | AD-MSC | BM-MSC |
|---|---|---|---|---|---|
| CD11 | Negative | Negative | Negative | Negative | Negative |
| CD14 | Negative | Negative | Negative | Negative | Negative |
| CD19 | Negative | Negative | Negative | Negative | Negative |
| CD34 | Negative | Negative | Negative | Negative | Negative |
| CD45 | Negative | Negative | Positive | Negative | Positive |
| CD73 | Positive | Positive | Positive | Positive | Positive |
| CD79a | Negative | Negative | Negative | Negative | Positive |
| CD90 | Positive | Positive | Positive | Positive | Positive |
| CD105 | Positive | Positive | Positive | Positive | Positive |
| HLA-DR | Negative | Negative | Negative | Negative | Negative |

Positive values are >90% and negative are <5%

### Immunomodulatory Potency Measures of AD-MSCs, BM-MSCs, P-MSCs, and UC-MSCs

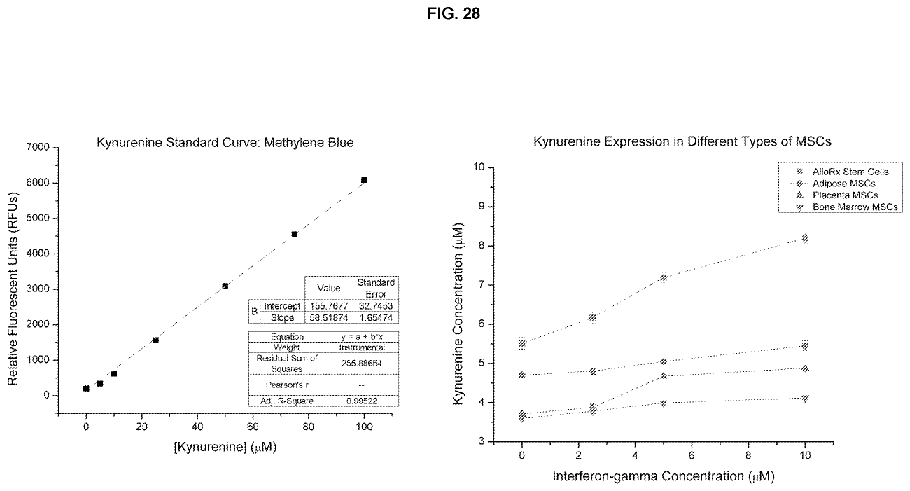

To compare immunomodulatory properties of MSCs from various sources, the activation of IDO by exposure to γ-IFN was determined on an equivalent cellular basis (FIG. 28). The γ-IFN-induced IDO activity was quantified by the conversion of tryptophan to kynurenine. Maximal IDO activity at 10 ng/ml γ-IFN was ˜4 fold greater in the isolated and expanded UC-MSCs versus other MSCs derived from other tissues. These results show greatest immunomodulatory cellular potency in expanded UC-MSCs followed by AD-MSCs, P-MSCs, and BM-MSCs. There was a significant difference in γ-IFN-induced IDO activity between the AD-MSCs, BM-MSCs, and P-MSCs compared to UC-MSCs with a p-value<0.005 by one-way ANOVA analysis (Graph Pad Prism™) of variance for significance of slope difference.

### Mitochondrial Function Analysis of AD-MSCs, BM-MSCs, P-MSCs, and UC-MSCs

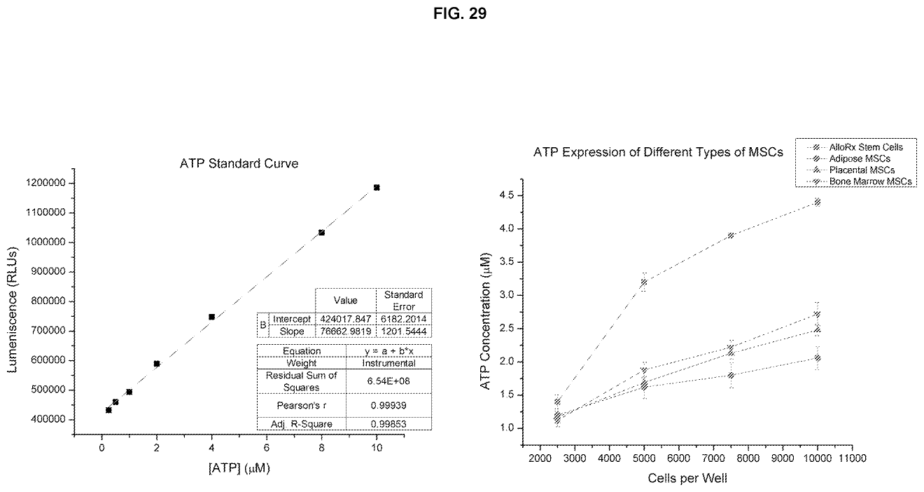

Potency was also measured by cell-specific ATP determination as previously used to determine potency of human HSCs & MSCs (Harper, H and Rich, IN, Methods Mol Biol. 2015; 1235:33; Deskins D, et al., Stem Cells Transl Med. 2013; 2:151) Relative luminescent units were converted to [ATP] using the ATP standard curve (Left panel, FIG. 29) and cellular ATP is shown as a function of cells per well (Right panel, FIG. 29). Cellular potency is measured by the slope of this relation (Harper, H and Rich, IN, Methods Mol Biol. 2015; 1235:33; Deskins D, et al., Stem Cells Transl Med. 2013; 2:151) and UC-MSCs showed greater potency than expanded AD-MSCs, BM-MSCs, and P-MSCs. There was a significant difference between the AD-MSCs, BM-MSCs, and P-MSCs compared to the isolated and expanded UC-MSCs with a p-value >0.05 by one-way ANOVA analysis (Graph Pad Prism™) of variance for significance of slope difference.

### Comparison of Cell Migration by AD-MSCs, BM-MSCs, P-MSCs, and UC-MSCs

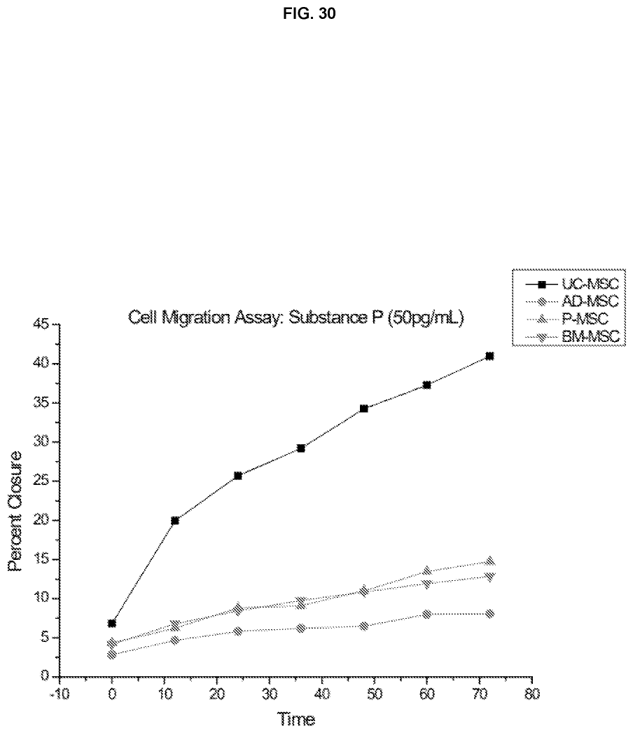

Since MSCs are well known to migrate to sites of inflammation, injury and to cancer stem cells, we compared the migration of AD-MSCs, P-MSCs, BM-MSCs and UC-MSCs in response to exposure to substance P, a multi-functional neuropeptide. The results show in FIG. 30 show the relative migration measured as a per-cent closure of the occluded plate region following exposure to 50 μg/ml substance P. UC-MSCs showed greatest closure at 50 pg/mL substance P (˜40% closure), while AD-MSC, P-MSC, and BM-MSC had a closure between 5-15%. These results were seen in several replicates (n=4).

### Cell Proliferation Analysis of AD-MSCs, BM-MSCs, P-MSCs, and UC-MSCs

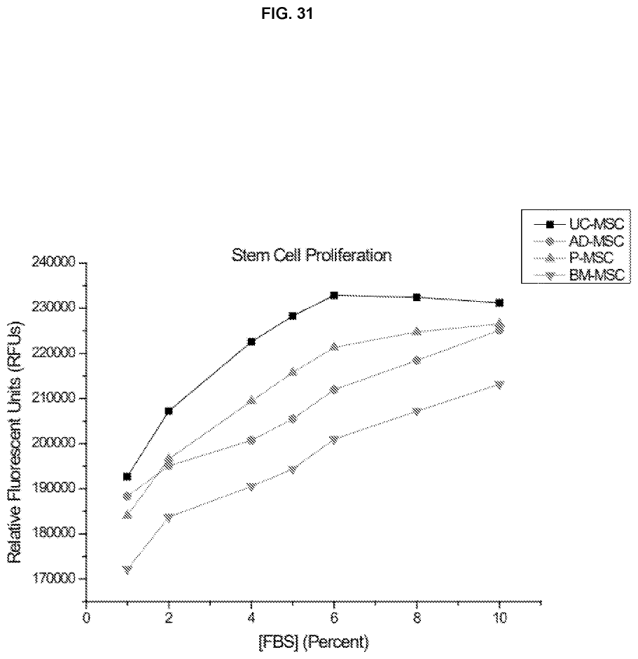

We also compared proliferation capacity of AD-MSCs, P-MSCs, BM-MSCs and UC-MSCs by quantifying cellular redox activity by a well-validated resazurin-based fluorometric assay. FIG. 31 shows the results of the comparison of proliferation by AD-MSCs, P-MSCs, BM-MSCs and UC-MSCs. The relative fluorescent difference at day 1 and 3 using Presto Blue as shown as a function of FBS content in a serum-free medium. These results were seen in several replicates (n=4). UC-MSC had a maximum effect of FBS at 6%, while no saturation was seen in the other cell lines.

### Comparison of Differentiation Capacity

We also compared functional differentiation of AD-MSCs, P-MSCs, BM-MSCs and UC-MSCs. First, we determined tri-lineage differentiation into adipocytes, chondrocytes and osteoblasts. We used standard methods that showed equivalent differentiation between the MSCs derived from adipose, placental, bone marrow and umbilical tissues (data not shown). We also investigated differentiation into neural stem cells and the results of IHC marker expression are shown in Table 11. The markers Nestin, 3PDGH, GLAST, β3-Tubulin, MAP2 & Neurofilament M are specific to neural stem cells (Wu, R, et al, Cell Biol Int 2013; 37: 812) and while the various MSCs tested were positive for most markers, the P-MSCs and AD-MSCs were negative for GLAST while this antigen was expressed on cells derived from UC-MSCs as well as the control NSCs (hNSC). This suggests a difference in differentiation capacity in that UC-MSCs can fully differentiate into the NSC phenotype while AD-MSCs and P-MSCs do not using our differentiation protocol. This does not necessarily indicate a lack of capacity of P-MSCs or AD-MSCs to differentiate into NSCs.

TABLE 11
IHC Marker Expression

| | | | | βIII- | | Neurofila- |
|---|---|---|---|---|---|---|
| | Nestin | 3PDGH | GLAST | Tubulin | MAP2 | ment M |
| AD-MSC | Positive | Positive | Negative | Positive | Positive | Positive |
| P-MSC | Positive | Positive | Negative | Positive | Positive | Positive |
| UC-MSC | Positive | Positive | Positive | Positive | Positive | Positive |
| hNSC | Positive | Positive | Positive | Positive | Positive | Positive |

### Discussion

We compared the cellular phenotype, potency, and functionality of expanded MSCs from different sources. Expanded MSCs were derived from lipoaspirate, bone marrow, placental decidua basalis, and Wharton's jelly of the umbilical cord. Our results showed expanded MSCs share universal properties, such as morphology, plastic adherence, and multi-lineage differentiation potential. We found variations between AD-MSCs, BM-MSCs, P-MSCs, and UC-MSCs in terms of growth rate, phenotypic characterization, potency, and functionality measurements.

We used quantitative assays to determine cell counts, viability, phenotype, potency by immune-modulatory and mitochondrial function, and functionality by migration and proliferation. Variability in measurement was minimized by careful adherence to standard procedures including processing, analysis, and expansion. Additionally, each assay was performed at the same passage to avoid variation due to differences in passage number (Javazon E H, et al, Exp Hematol. 2004; 32:414).

International criteria of MSC identity was determined by flow cytometry according to ISCT standards (Dominici M, et al. Cytotherapy. 2006; 8: 315). Placental MSCs and bone-marrow MSCs did not achieve ISCT criterion values of CD45 and CD79a. The increased expression of CD45 in P-MSCs may be due to associated leukocytes and CD79a from residual B-cells.

Cellular potency is an important assessment of stem cells for clinical applications. We used quantitative assessment of mitochondrial function and immunosuppression as measures of cellular potency. Since MSCs are intrinsically immunosuppressive in nature, they can support graft survival and other clinical effects based on immunosuppression (Liu, R, et al., Stem Cells Dev 2013; 22:1053; Wang, L T, et al, J Biomed Sci 2016; 23: 76). However, the failure of MSCs to elicit immunosuppression is likely due to immune enhancing effects of MSCs triggered by proinflammatory cytokines, educed NO, etc while IDO expression induces immunosuppressive effects of MSCs. IDO has been proposed as a molecular switch to induce immunosuppression in MSCs (Li, W et al, Cell Death & Differentiation 19: 1505, 2012). We thus determined cellular potency by quantitation of γ-IFN induced IDO activity. The results showed maximum immunomodulatory potency in UC-MSCs, which was significantly greater than MSCs sourced from other tissues (FIG. 28). This compares with other studies. Wang, Q, et al, (Human Vaccin & Immunother 12: 85, 2016) compared fetal BM derived MSCs, AD-MSCs and MSCs derived from Wharton's jelly of the umbilical cord. They found comparable phenotype, proliferation, clonality and that γ-IFN induced IDO expression was greatest in UC-MSCs, supporting our findings as well. Kim, J H, et al, (Stem Cells International, 2018: 8429042), also showed superior immunosuppression and minimal HLA-DR expression in UC-MSCs compared to AD-MSCs and periodontal ligament-derived MSCs. Other reports of the comparison of MSC from various tissue sources also support biological advantages of UC-MSCs (Riordan, N H et al, J Transl Med 2018; 16: 57; Najar, M, et al, Cell Immunol. 2010; 264:171; Weiss, M L, et al, Stem Cells 2006; 24: 781; Arutyunyan, I, et al, Stem Cell International 2016; 2016: 6901286) Jin, H J, et al, (Int. J. Mol Sci 2013; 14: 17986), showed superior proliferation and anti-inflammatory properties of UC-MSCs compared to BM-MSCs and AD-MSCs.

Expanded MSCs showed measurable levels of cell-specific ATP content. However, cell-specific ATP expression was significantly higher in UC-MSCs supporting the assertion that they are the most potent type of MSC. Other studies have shown that ATP expression correlates with therapeutic outcomes in the transplantation of hematopoietic stem cells (Deskins D, et al, Stem Cells Transl Med. 2013; 2:151; Rich, IN Stem Cell Transl Med 2015; 4: 967).

Numerous clinical trials have been conducted and are presently ongoing for various MSC preparations (Carralho E, et al. Regen. Med. 2015; 10:1025; Freitag J, et al. BMC Musculoskeletal Disorders 2016; 17: 230; Neirinckx V, et al. Stem Cell Trans. Med 2013; 2: 284; Munir H and McGettrick, HM Stem Cells Dev. 2015; 24:2091). From the results reported here it would be expected that expanded UC-MSCs exhibit greater therapeutic benefit than other impure sources of MSCs such as bone marrow aspirate and stromal vascular fraction. Direct clinical comparisons from various sourced MSCs are lacking.

Mechanisms of stem cell therapy include paracrine effects from stem cell-derived biological factors eliciting anti-inflammatory & neural protective effects, differentiation of stem cells into other cellular lineages, and intercellular communication through tunneling nanotubes.

### Conclusion

Our results show bio-similarity between stem cells derived from adipose, bone marrow, placental and umbilical cord tissues regarding expansion, trilineage differentiation, and phenotypic characterization by flow cytometry according to the ISCT definition of MSCs. While all sources of MSCs also exhibited activity in potency assays including quantitative assessment of mitochondrial function and immunosuppression, cell migration and proliferation, there were clear differences. Our results revealed significant superiority of UC-derived MSCs as was also found in similar studies performed in several other laboratories. Age of the cells may be a factor in the overall performance of MSCs. Furthermore, the capacity to differentiate into neural stem cells varied between MSC derived from UC, adipose and placental tissues with UC derived MSCs expressing all NSC markers while adipose and placental-derived MSCs did not express GLAST under identical conditions. Thus, while MSCs from various tissues show similarity, there are also multiple characteristics of umbilical cord MSCs significantly superior to those derived from adipose, bone marrow or placental tissues. This suggests that UC-MSCs may also exhibit superior therapeutic benefits.

### Example 18: Treatment of PASC with Genetically Modified Stem Cells

A subject experiencing brain fog and a loss of their sense of smell is diagnosed with Post-Acute Sequelae of SARS CoV-2 (PASC) by a clinician who previously detected the SARS-CoV-2 antigen by a lateral flow assay about 4 weeks ago. After four weeks of experiencing these symptoms, the subject can be administered a 6 mL pharmaceutical composition containing 1×10⁸ modified stem cells (e.g., UC-MSCs) in PRIME-XV® MSC FreeziS DMSO-Free cryopreservation medium and a pharmaceutical diluent. The modified stem cells (e.g., UC-MSCs) are from a subject that does not have PASC (e.g., the UC-MSCs are autologous) and have been genetically modified so that an additional copy of fibroblast growth factor 2 (FGF-2) is being expressed by a nucleic acid vector that was previously transfected into the stem cells (e.g., UC-MSCs). Administration of the pharmaceutical composition to the subject occurs intravenously over the course of 20 minutes. Concurrently with the administration of the pharmaceutical composition, the subject receives about 400 mg of the HDAC1 inhibitor, vorinostat, by tablet. The subject can continue to receive treatment with the pharmaceutical composition every six months. After two treatments over a 12 month period, the subject is examined for restoration of their sense of smell. Regaining the sense of smell indicates that the treatment is working.

With a third treatment, the subject can be administered a different pharmaceutical composition containing 2.5×10⁶ modified stem cells (e.g., NSCs) per kilogram (kg) of the subject's body weight in PRIME-XV® MSC FreeziS DMSO-Free cryopreservation medium and a pharmaceutical diluent, totaling a 1.5 mL volume. The modified stem cells (e.g., NSCs) can be obtained from a subject that does not have PASC (e.g., the NSCs are autologous) and have been genetically modified so that two copies of FGF-2 are being expressed by a circular RNA that was previously transfected into the stem cells (e.g., NSCs). This pharmaceutical composition can be administered to the subject intravenously over the course of 2 minutes. The subject may continue to receive this treatment every six months as needed.

### Example 19: Treating PASC with UC-MSCs

A subject with PASC can be treated according to the methods described herein. The subject may be diagnosed by a clinician, such as by determining if the subject has experienced a symptom of PASC (e.g., cognitive impairment, joint pain, muscle pain, chest pain, stomach pain, headache, tachycardia, numbness, diarrhea, brain fog, fatigue, sleep impairment, dizziness when standing, shortness of breath, fever, skin rash, mood changes, loss of smell, exercise intolerance, or loss of taste) for more than 4 weeks. The subject may be administered (e.g., within a week or more of diagnosis) a 3 mL pharmaceutical composition containing a total of 9.5×10⁷ UC-MSCs in non-DSMO cryopreservation medium. The UC-MSCs can be produced from a subject that does not have PASC (e.g., the UC-MSCs are allogeneic).

Prior to administration of the pharmaceutical composition, the subject may be treated with romidepsin, such as in an amount of about 14 mg/m², by intravenous infusion (e.g., over a 4-hour period). After the infusion, the subject's serum levels can be assessed to confirm a level of romidepsin of, e.g., about 500 μM. Administration of the pharmaceutical composition to the subject can be by intravenous infusion, such as over the course of 45 minutes. The subject may continue to receive treatment with the pharmaceutical composition as needed, such as once every three months. After 12 months, the subject exhibits a blood urea nitrogen (BUN) level of about 8-25 mEq/L; a creatine level of about 0.5-1.5 mEq/L; an aspartate transaminase (AST) level of about 8-33 U/L; an alanine aminotransferase (ALT) level of about 4-36 U/L; and an alkaline phosphatase level of about 25-115 U/L, thereby indicating that the treatment is working; treatment may then be paused or halted. Treatment may resume at any point if the subject experiences an abnormal clinical laboratory measurement, such as a BUN level greater than 25 mEq/L; a creatine level greater than 1.5 mEq/L; an AST level greater than 33 U/L; an ALT) level greater than 36 U/L; and an alkaline phosphatase level greater than 115 IU/L.

### Example 20: Treating PASC with Isolated Exosomes

A subject with PASC can be treated (e.g., within one year of diagnosis) according to the methods described herein. The subject may be diagnosed by a clinician, such as by determining if the subject has an abnormal measurement during an arterial blood gas (ABG) test, such as an arterial blood pH level outside the range of 7.37-7.44; a partial pressure of carbon dioxide (pCO₂) outside the range of 34-43 mm Hg; a partial pressure of oxygen (pO₂) and fraction of inspired oxygen (FiO₂) ratio (pO₂/FiO₂) of less than 300; or a positive end-expiratory pressure (PEEP) of less than 30 cm H₂O.

Alternatively, the diagnosis may be based presence of any of the following symptoms: cognitive impairment, joint pain, muscle pain, chest pain, stomach pain, headache, tachycardia, numbness, diarrhea, brain fog, fatigue, sleep impairment, dizziness when standing, shortness of breath, fever, skin rash, mood changes, loss of smell, exercise intolerance, and loss of taste.

The subject may be administered a 1 mL pharmaceutical composition containing 5×10⁹ isolated exosomes per mL in a saline solution. The isolated exosomes may be about 80 to about 200 nm (e.g., 72 nm, 80 nm, 90 nm, 100 nm, 110 nm, 120 nm, 130 nm, 140 nm, 150 nm, 160 nm, 170 nm, 180 nm, 190 nm, 200 nm, 210 nm, or 220 nm) in size and express CD9, CD63, CD81, CD44, CD29, and CD142. Administration of the pharmaceutical composition to the subject may occur by IV drip over the course of about 60 minutes (e.g., 54 minutes to 66 minutes). The subject may continue to receive treatment with the pharmaceutical composition every three months (e.g., every 27, 28, 29, 30, 31, 32, or 33 days). After two treatments over a 6 month period (e.g., 54, 55, 56, 57, 58, 59, 60, 61, 62, or 63 days), the subject's ABG test shows improvements, suggesting that the treatment is working.

On the fifth treatment, the subject with PASC may be administered a 10 mL pharmaceutical composition containing 1×10⁸ modified MSCs (e.g., UC-MSCs or NSCs) in a cryopreservation medium, and a pharmaceutical diluent. The modified MSCs (e.g., UC-MSCs or NSCs) may be from a subject that does not have PASC (e.g., the MSCs are autologous) and have been genetically modified (e.g., transfected with genetic material) so that three copies of FGF-2 is being expressed by e.g., a circular RNA that was previously transfected into the MSCs (e.g., UC-MSCs or NSCs). Additionally, the genetically modified MSCs (e.g., UC-MSCs or NSCs) may be cultured in 500 nM of a nutraceutical (e.g., reservatrol, curcumin, and/or quercetin) for about six days prior to administration. This pharmaceutical composition may be administered to the subject by intravenous infusion over the course of 50 minutes. The subject may continue to receive this treatment as needed, such as every six months for the life of the subject or until the subjects ABG test indicates a pH level within the range of 7.35-7.4; a pCO₂ within the range of 37-45 mm Hg; a pO₂/FiO₂ greater than 300; or a PEEP of less than 30 cm H₂O.

### Example 21: Treating PASC with UC-MSC Secretome-Conditioned Cell Culture Medium

A subject experiencing fatigue and brain fog for 5 weeks after receiving a SARS-CoV-2 vaccine may be diagnosed with PASC by a clinician who, at the 5^{th} week, determines one of the following abnormal clinical laboratory measurements in the subject: a lactic acid level greater than 4.0 mmol/L; a procalcitonin level greater than 0.25 ng/mL; a lactate dehydrogenase (LDH) level greater than 240 U/L; a ferritin level greater than 284 ng/mL; a c-reactive protein (CRP) level greater than 1.0 mg/dL; or a d-dimer level greater than 500 ng/mL. The subject may then be administered (e.g., within one day of diagnosis) a 10 mL pharmaceutical composition containing stem cell (e.g., MSC, UC-MSC, or NSC) secretome-conditioned cell culture medium. The composition may contain exosomes that express CD9, CD63, CD81, CD44, CD29, and CD142 and various proteins (e.g., GM-CSF, MIP-3a, IL-6, IL-8, MIP-1, and fractalkine) each at a concentration of at least about 100 pg/mL (e.g., 90 pg/mL to 1000 pg/mL).

Administration of the pharmaceutical composition to the subject may occur intravenously (e.g., over the course of 30 minutes). The subject may continue to receive treatment with the pharmaceutical composition as needed, such as once every month. After eight treatments over an 8 month period, the subject's clinical laboratory measurements return to baseline (e.g., the subject has a lactic acid level of about 2-4 mM; a procalcitonin level of about 0.1-0.25 ng/mL; an LDH level of about 50-240 U/L; a ferritin level of about 30-284 ng/mL; a CRP level of about 0.3-1.0 mg/dL; and/or a d-dimer level of less than 500 ng/mL) and the subject reports little to no fatigue or brain fog symptoms.

### Example 22: Treating PASC with Exosome-Depleted Stem Cell Secretome-Conditioned Cell Culture Medium

A subject with COVID for over 4 weeks and with symptoms of cognitive impairment, joint pain, muscle pain, and chest pain and a blood urea nitrogen (BUN) level of 30 mEq/L may be diagnosed with PASC by a clinician. The subject may be administered a 2 mL pharmaceutical composition containing exosome-depleted stem cell secretome-conditioned cell culture medium. The composition may not contain any exosomes between 140-200 nm in diameter; however, the composition may contain GM-CSF, MIP-3a, IL-6, and IL-8 each at a concentration of about 400-500 pg/mL (e.g., 450 pg·mL) and MIP-1 and fractalkine at a concentration of 50-100 pg/mL (e.g., 75 pg/mL).

Administration of the pharmaceutical composition to the subject having PASC may occur intravenously (e.g., over the course of 35 minutes). The subject can continue to receive treatment with the pharmaceutical composition as needed, e.g., once every three months or once every six months. After eight treatments over a 24 month period, if the subject reports no symptoms of cognitive impairment, joint pain, muscle pain, and chest pain and has a measured a BUN level of about 19 mEq/L, treatment can be considered complete.

### Example 23: Treating PASC with Resveratrol, Curcumin, and Quercetin

A subject experiencing brain fog and a loss of their sense smell is diagnosed with PASC by a clinician who previously detected the SARS-CoV-2 antigen by a lateral flow assay about 4 weeks ago. Further, the subject's diagnosis is corroborated by an abnormal measurement during an arterial blood gas (ABG) test, such as an arterial blood pH level outside the range of 7.37-7.44; a partial pressure of carbon dioxide (pCO₂) outside the range of 34-43 mm Hg; a partial pressure of oxygen (pO₂) and fraction of inspired oxygen (FiO₂) ratio (pO₂/FiO₂) of less than 300; or a positive end-expiratory pressure (PEEP) of less than 30 cm H₂O.

The subject may be administered (e.g., within 2 days of diagnosis) a 2 mL pharmaceutical composition containing 253 nM of resveratrol, 400 nM of curcumin, and 500 nM of quercetin in a pharmaceutical excipient containing piperine. Administration of the pharmaceutical composition to the subject may occur intravenously or by oral administration (e.g., over the course of 30 minutes). The subject may continue to receive treatment with the pharmaceutical composition as needed, such as once every day. After eight treatments over about 48 days, the subject's clinical laboratory measurements return to baseline (e.g., the subject's blood has a pH level within the range of 7.35-7.45, such as about 7.435; a pCO₂ within the range of 37-45 mm Hg, such as about 43.3 mm Hg; a pO₂/FiO₂ greater than 300, such as about 316; and a PEEP of greater than 30 cm H₂O) and the subject reports little to no fatigue or brain fog symptoms.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations following, in general, the principles and including such departures from the invention that come within known or customary practice within the art to which the invention pertains and may be applied to the essential features hereinbefore set forth, and follows in the scope of the claims.

Other embodiments are within the claims.

1. A method of treating a subject with Post-Acute Sequelae of SARS CoV-2 (PASC) comprising administering to the subject an effective amount of a composition comprising:
(a) a plurality of stem cells;
(b) a plurality of isolated exosomes about 80-200 nanometers (nm) in diameter, wherein the isolated exosomes are derived from the stem cells;
(c) stem cell secretome-conditioned cell culture medium;
(d) exosome-depleted stem cell secretome-conditioned cell culture medium; and/or
(e) resveratrol, curcumin, and/or quercetin.

2. The method of claim 1, wherein the composition is administered intravenously, intra-articularly, intramuscularly, intranasally, intrathecally, or by oral administration.

3. The method of claim 2, wherein the composition is administered intravenously.

4. The method of claim 3, wherein the composition is administered intravenously by infusion.

5. The method of any one of claims 1-4, wherein the composition is administered in a volume of about 1 milliliter (mL) to about 15 mL.

6. The method of claim 5, wherein the composition is administered in a volume of about 1 mL to about 10 mL.

7. The method of claim 6, wherein the composition is administered in a volume of about 1 mL, about 2 mL, about 3 mL, about 4 mL, about 5 mL, or about 6 mL.

8. The method of any one of claims 1-7, wherein the composition is administered in a bolus or is administered over a period of about 1 minute to about 1 hour.

9. The method of claim 8, wherein the composition is administered over a period of about 1 minute to about 30 minutes.

10. The method of claim 9, wherein the composition is administered over a period of about 1 minute to about 10 minutes.

11. The method of any one of claims 1-10, wherein the composition is administered at a frequency of once every one, two, three, four, five, six, seven, eight, nine, ten, eleven, or twelve months.

12. The method of claim 11, wherein the composition is administered at a frequency of once every six to twelve months.

13. The method of claim 12, wherein the composition is administered at a frequency of once every six months.

14. The method of any one of claims 1-10, wherein the composition comprises resveratrol, curcumin, and/or quercetin and is administered at a frequency of once every one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty days.

15. The method of claim 14, wherein the composition comprising resveratrol, curcumin, and/or quercetin is administered at a frequency of once every six days.

16. The method of any one of claims 11-15, wherein the composition is administered at said frequency for up to one month, six months, nine months, one year, or until resolution of one or more symptoms of PASC.

17. The method of any one of claims 1-16, wherein the stem cells are modified to increase an expression level of sirtuin 1 (Sirt-1), C—X—C motif chemokine receptor 4 (CXCR4), heat shock protein 70 (HSP70), octamer-binding transcription factor 3/4 (Oct 3/4), and/or fibroblast growth factor 21 (FGF-21) relative to unmodified stem cells.

18. The method of any one of claims 1-17, wherein the stem cells comprise a nucleic acid vector, plasmid, circular RNA, or mRNA molecule comprising a nucleotide sequence encoding Sirt-1, CXCR4, HSP70, Oct 3/4, FGF-21, fibroblast growth factor 2 (FGF-2), and/or substance P.

19. The method of any one of claims 1-18, wherein the method further comprises, prior to administering the composition, contacting the stem cells with a stem cell activating agent.

20. The method of claim 19, wherein the stem cell activating agent is selected from the group consisting of a histone deacetylase 1 (HDAC1) inhibitor, a glycogen synthase kinase-3β (GSK-3Bβ) inhibitor, a neurokinin-1 (NK-1) receptor agonist, a fibroblast growth factor (FGF) protein, a nutraceutical, and lithium (Li).

21. The method of claim 20, wherein:
(a) the HDAC1 inhibitor is selected from the group consisting of romidepsin (FK-228), vorinostat, romidepsin, belinostat, panobinostat, valproic acid (VPA), entinostat, curcumin, quercetin, and RG2833;
(b) the GSK-3Bβ inhibitor is selected from the groups consisting of indirubin-3′-oxime, laduviglusib (CHIR-99821), and KY19382;
(c) the NK-1 receptor agonist is substance P;
(d) the FGF protein is FGF-2; and/or
(d) the nutraceutical is selected from the group consisting of resveratrol, curcumin, and quercetin.

22. The method of claim 21, wherein:
(a) contacting of the stem cells is with curcumin at a concentration of about 0.1 μM to about 1 μM;
(b) contacting of the stem cells is with curcumin at a concentration of about 0.1 μM to about 1 μM and quercetin at a concentration of about 0.1 μM to about 1 μM;
(c) contacting of the stem cells is with VPA at a concentration of about 20 μM to about 100 PM;
(d) contacting of the stem cells is with Li at a concentration of about 10 μM to about 200 PM;
(e) contacting of the stem cells is with VPA at a concentration of about 10 μM to about 125 μM and Li at a concentration of about 200 μM;
(f) contacting of the stem cells is with VPA at a concentration of about 20 μM to about 250 μM and curcumin at a concentration of about 100 nM to about 1 μM;
(g) contacting of the stem cells is with substance P at a concentration of about 1 nM to about 20 nM;
(h) contacting of the stem cells is with FGF-2 at a concentration of about 3 ng/mL to 10 ng/mL; or
(i) contacting of the stem cells is with curcumin at a concentration of about 500 nM and resveratrol at a concentration of about 50 nM to about 500 nM.

23. The method of claim 22, wherein:
(a) contacting of the stem cells is with curcumin at a concentration of about 500 nM;
(b) contacting of the stem cells is with curcumin at a concentration of about 500 nM and with quercetin at a concentration of about 317 nM;
(c) contacting of the stem cells is with VPA at a concentration of about 38 μM;
(d) contacting of the stem cells is with Li at a concentration of about 79 μM;
(e) contacting of the stem cells is with VPA at a concentration of about 37 μM and Li at a concentration of about 200 μM;
(f) contacting of the stem cells is with VPA at a concentration of about 250 μM and curcumin at a concentration of about 500 nM;
(g) contacting of the stem cells is with substance P at a concentration of about 2.5 nM;
(h) contacting of the stem cells is with FGF-2 at a concentration of about 4.2 ng/mL; or
(i) contacting of the stem cells is with curcumin at a concentration of about 500 nM and resveratrol at a concentration of about 253 nM.

24. The method of claim 20 or 21, wherein the HDAC1 inhibitor is at a concentration of about 1 nM to about 10 μM.

25. The method of claim 24, wherein the HDAC1 inhibitor is at a concentration of about 100 nM to about 1 μM.

26. The method of claim 25, wherein the HDAC1 inhibitor is at a concentration of about 400 nM to about 600 nM.

27. The method of claim 26, wherein the HDAC1 inhibitor is at a concentration of about 500 nM.

28. The method of claim 20 or 21, wherein the GSK-3Bβ inhibitor is at a concentration of about 1 nM to about 10 μM.

29. The method of claim 28, wherein the GSK-3Bβ inhibitor is at a concentration of about 100 nM to about 1 μM.

30. The method of claim 29, wherein the GSK-3Bβ inhibitor is at a concentration of about 400 nM to about 600 nM.

31. The method of claim 30, wherein the GSK-3Bβ inhibitor is at a concentration of about 500 nM.

32. The method of claim 20 or 21, wherein the nutraceutical is at a concentration of about 50 nM to about 500 nM.

33. The method of claim 32, wherein the nutraceutical is at a concentration of about 500 nM.

34. The method of any one of claims 19-33, wherein the contacting is for about 1 day to about 6 weeks.

35. The method of claim 34, wherein the contacting is for about 1-3 weeks.

36. The method of claim 35, wherein the contacting is for about 2 weeks.

37. The method of claim 36, wherein the contacting is for about 5 days.

38. The method of any one of claims 19-37, wherein the stem cell activating agent increases Sirt-1, CXCR4, HSP70, Oct 3/4, and/or FGF-21 expression levels in the stem cells relative to stem cells that are not in contact with the stem cell activating agent.

39. The method of claim 38, wherein the expression level of Sirt-1 is increased in the stem cells by about 200-fold to about 300-fold, the expression level of CXCR4 is increased in the stem cells by about 10-fold to about 20-fold, the expression level of HSP70 is increased in the stem cells by about 2-fold to about 20-fold, the expression level of Oct 3/4 is increased in the stem cells by about 10-fold to about 20-fold, and/or the expression level of FGF-21 is increased in the stem cells by about 5-fold to about 20-fold relative to stem cells that are not in contact with the stem cell activating agent.

40. The method of any one of claims 19-39, wherein the stem cell activating agent increases proliferation and/or migration of the stem cells relative to stem cells that are not in contact with the stem cell activating agent.

41. The method of any one of claims 1-40, wherein the composition comprises:
(a) about 5×10⁷ to about 1×10⁸ of the stem cells;
(b) a concentration of about 5×10⁹ to about 5×10¹⁰ of the exosomes per mL, wherein optionally said exosomes express cluster of differentiation CD9, CD63, CD81, CD29, CD44 and/or CD144;
(c) about 100 pg/mL to about 5000 pg/mL of granulocyte-macrophage colony-stimulating factor (GM-CSF), macrophage inflammatory protein (MIP)-3 alpha (MIP-3a), IL-6, and IL-8 and/or about 10 pg/mL to about 1000 pg/mL of fractalkine and MIP-1;
(d) about 50 nM to about 500 nM of resveratrol, curcumin, or quercetin; and/or
(e) a pharmaceutically acceptable carrier, excipient, or diluent,
wherein, optionally, the composition does not contain DMSO.

42. The method of claim 41, wherein the excipient comprises piperine.

43. The method of claim 41 or 42, wherein the composition further comprises:
(a) a cryopreservation medium;
(b) a basal medium; and/or
(c) a saline solution.

44. The method of claim 43, wherein:
(a) the cryopreservation medium is PRIME-XV® MSC FreezIS DMSO-Free medium; and/or
(b) the basal medium is MCDB-131.

45. The method of any one of claims 41-44, wherein the composition comprises about 7×10⁷ to about 1×10⁸ of the stem cells.

46. The method of any one of claims 41-45, wherein the composition comprises about 1×10⁸ of the stem cells.

47. The method of any one of claims 1-46, wherein the stem cells are mesenchymal stem cells (MSCs) or neural stem cells (NSCs).

48. The method of claim 47, wherein the MSCs are umbilical cord-derived MSCs (UC-MSCs).

49. The method of claim 48, wherein the UC-MSCs are ALLORX STEM CELLS®.

50. The method of claim 47, wherein the NSCs are nasal epithelium-derived MSCs.

51. The method of any one of claims 41-50, wherein the pharmaceutical composition comprises a concentration of about 1×10¹⁰ to about 5×10¹⁰ of the isolated exosomes per mL.

52. The method of any one of claims 1-51, wherein:
(a) the exosomes express CD9, CD63, and CD81;
(b) the exosomes express CD44, CD29, and CD142; and/or
(c) the exosomes do not express CD45, CD11b, CD14, CD19, CD34, CD79a, CD126, and human leukocyte antigen-DR isotype (HLD-DR).

53. The method of any one of claims 41-52, wherein the pharmaceutical composition comprises about 100 pg/mL to about 1000 pg/mL of GM-CSF, MIP-3a, IL-6, and IL-8 and/or about 10 pg/mL to about 100 pg/mL of fractalkine and MIP-1.

54. The method of claim 53, wherein the pharmaceutical composition comprises about 1000 pg/mL of GM-CSF, MIP-3a, IL-6, and IL-8 and/or about 100 pg/mL of fractalkine and MIP-1.

55. The method of any one of claims 1-54, wherein, prior to administration of the composition, the method comprises administering a stem cell activating agent to the subject.

56. The method of claim 55, wherein the stem cell activating agent is selected from the group consisting of a HDAC1 inhibitor, a GSK-3Bβ inhibitor, an NK-1 receptor agonist, an FGF protein, a nutraceutical, Li, and a neural cell adhesion molecule (NCAM) modulator.

57. The method of claim 56, wherein:
(a) the HDAC1 inhibitor is selected from the group consisting of romidepsin (FK-228), vorinostat, romidepsin, belinostat, panobinostat, VPA, entinostat, curcumin, quercetin, and RG2833;
(b) the GSK-3Bβ inhibitor is selected from the groups consisting of indirubin-3′-oxime, laduviglusib (CHIR-99821), and KY19382;
(c) the NK-1 receptor agonist is substance P;
(d) the FGF protein is FGF-2; and/or
(d) the nutraceutical is resveratrol, curcumin, or quercetin; and/or
(e) the NCAM modulator is:
(i) N-butylmannosamine; or
(ii) an inhibitory nucleic acid molecule that targets ST8 alpha-N-acetyl-neuraminide alpha-2,8-sialyltransferase 1 (ST8Sial) and/or ST8 alpha-N-acetyl-neuraminide alpha-2,8-sialyltransferase 5 (ST8SiaV).

58. The method of claim 56 or 57, wherein the HDAC1 inhibitor is administered to the subject in an amount sufficient to achieve a serum concentration of the HDAC1 inhibitor of about 0.1 nM to about 1000 nM.

59. The method of claim 58, wherein the HDAC1 inhibitor is administered to the subject in an amount sufficient to achieve a serum concentration of the HDAC1 inhibitor of about 500 nM.

60. The method of claim 56 or 57, wherein the GSK-3Bβ inhibitor is administered to the subject in an amount sufficient to achieve a serum concentration of the GSK-3Bβ inhibitor of about 0.1 nM to about 1000 nM.

61. The method of claim 60, wherein the GSK-3Bβ inhibitor is administered to the subject in an amount sufficient to achieve a serum concentration of the GSK-3Bβ inhibitor of about 500 nM.

62. The method of claim 56 or 57, wherein the NK-1 receptor agonist is administered to the subject in an amount sufficient to achieve a serum concentration of the NK-1 receptor agonist at a concentration of about 1 nM to about 5 nM.

63. The method of claim 62, wherein the NK-1 receptor agonist is administered to the subject in an amount sufficient to achieve a serum concentration of the NK-1 receptor agonist of about 2.5 nM.

64. The method of claim 56 or 57, wherein the FGF protein is administered to the subject in an amount sufficient to achieve a serum concentration of the FGF protein of about 3 ng/mL to about 10 ng/mL.

65. The method of claim 64, wherein the FGF protein is administered to the subject in an amount sufficient to achieve a serum concentration of the FGF protein of about 4.2 ng/mL.

66. The method of claim 56 or 57, wherein the nutraceutical is administered to the subject in an amount sufficient to achieve a serum concentration of the nutraceutical of about 50 nM to about 500 nM.

67. The method of claim 66, wherein the nutraceutical is administered to the subject in an amount sufficient to achieve a serum concentration of the nutraceutical of about 500 nM.

68. The method of claim 56 or 57, wherein N-butylmannosamine is administered to the subject in an amount sufficient to achieve a serum concentration of N-butylmannosamine of about 1 mM to about 50 mM.

69. The method of claim 57, wherein the inhibitory nucleic acid molecule is a small interfering RNA (siRNA), an anti-sense oligonucleotide (ASO), a short hairpin RNA (shRNA), a micro RNA (miRNA), or a double stranded RNA (dsRNA).

70. The method of any one of claims 55-69, wherein the method comprises administering the stem cell activating agent to the subject concurrently with or following administration of the composition.

71. The method of claim 70, wherein the stem cell activating agent is selected from the group consisting of an HDAC1 inhibitor, a GSK-3Bβ inhibitor, an NK-1 receptor agonist, an FGF protein, a nutraceutical, Li, and an NCAM modulator.

72. The method of claim 71, wherein:
(a) the HDAC1 inhibitor is selected from the group consisting of romidepsin (FK-228), vorinostat, romidepsin, belinostat, panobinostat, VPA, entinostat, curcumin, quercetin, and RG2833;
(b) the GSK-3Bβ inhibitor is selected from the groups consisting of indirubin-3′-oxime, laduviglusib (CHIR-99821), and KY19382;
(c) the NK-1 receptor agonist is substance P;
(d) the nutraceutical is resveratrol, curcumin, and/or quercetin; and/or
(e) the NCAM modulator is:
(i) N-butylmannosamine; or
(ii) an inhibitory nucleic acid molecule that targets ST8Sial and/or ST8SiaV.

73. The method of claim 71 or 72, wherein the HDAC1 inhibitor is administered to the subject in an amount sufficient to achieve a serum concentration of the HDAC1 inhibitor of about 0.1 nM to about 1000 nM.

74. The method of claim 73, wherein the HDAC1 inhibitor is administered to the subject in an amount sufficient to achieve a serum concentration of the HDAC1 inhibitor of about 500 nM.

75. The method of claim 71 or 72, wherein the GSK-3Bβ inhibitor is administered to the subject in an amount sufficient to achieve a serum concentration of the GSK-3Bβ inhibitor of about 0.1 nM to about 1000 nM.

76. The method of claim 75, wherein the GSK-3Bβ inhibitor is administered to the subject in an amount sufficient to achieve a serum concentration of the GSK-3Bβ inhibitor of about 500 nM.

77. The method of claim 71 or 72, wherein the NK-1 receptor agonist is administered to the subject in an amount sufficient to achieve a serum concentration of the NK-1 receptor agonist of about 1 nM to about 5 nM.

78. The method of claim 77, wherein the NK-1 receptor agonist is administered to the subject in an amount sufficient to achieve a serum concentration of the NK-1 receptor agonist of about 2.5 nM.

79. The method of claim 71 or 72, wherein the FGF protein is administered to the subject in an amount sufficient to achieve a serum concentration of the FGF protein of about 3 ng/mL to about 10 ng/mL.

80. The method of claim 79, wherein the FGF protein is administered to the subject in an amount sufficient to achieve a serum concentration of the FGF protein of about 4.2 ng/mL.

81. The method of claim 71 or 72, wherein the nutraceutical is administered to the subject in an amount sufficient to achieve a serum concentration of the nutraceutical of about 50 nM to about 500 nM.

82. The method of claim 81, wherein the nutraceutical is administered to the subject in an amount sufficient to achieve a serum concentration of the nutraceutical of about 500 nM.

83. The method of claim 71 or 72, wherein N-butylmannosamine is administered to the subject in an amount sufficient to achieve a serum concentration of N-butylmannosamine of about 1 mM to about 50 mM.

84. The method of claim 72, wherein the inhibitory nucleic acid molecule is an siRNA, an ASO, an shRNA, a miRNA, or a dsRNA.

85. The method of claim 72, wherein the subject is administered:
(a) vorinostat in an amount of about 400 mg and/or in an amount sufficient to achieve geometric mean values of a maximum plasma concentration (Cₘₐₓ) and an area under the plasma concentration versus time curve (AUC_{0-inf}) of about 1.2±0.53 μM and about 6.0±2.0 μM*hr, respectively;
(b) romidepsin in an amount of about 14 mg/m² IV over a 4-hour period, such as on days 1, 8, and 15 of a 28-day cycle and/or in an amount sufficient to achieve geometric mean values of a maximum plasma concentration (Cₘₐₓ) and an area under the plasma concentration versus time curve (AUC_{0-inf} of about 377 ng/mL and about 1549 ng*hr/mL, respectively;
(c) belinostat in an amount of about 1000 mg/m² over a 30 minute period, such as on days 1-5 of a 21-day cycle;
(d) panobinostat in an amount of about 20 mg every other day, such as on days 1, 3, 5, 8, 10, and 12 of a 21-day cycle;
(e) valproic acid in an amount of about 10 to 60 mg/kg/day;
(f) entinostat in an amount of about 2 mg/m² to about 12 mg/m² per day;
(g) curcumin in an amount of about 1 g to about 8 g per day;
(h) quercetin in an amount of about 250 mg to about 1000 mg per day; and/or
(i) RG2833 in an amount of about 30 mg to about 240 mg per day.

86. The method of any one of claims 1-85, wherein the subject exhibits one or more symptoms of PASC, wherein optionally the one or more symptoms are present in the subject for at least four weeks prior to administration of the composition.

87. The method of claim 86, wherein the one or more symptoms of PASC results from an administration of a SARS-CoV-2 vaccine.

88. The method of claim 86 or 87, wherein the one or more symptoms are selected from the group consisting of: cognitive impairment, joint pain, muscle pain, chest pain, stomach pain, headache, tachycardia, numbness, diarrhea, brain fog, fatigue, sleep impairment, dizziness when standing, shortness of breath, fever, skin rash, mood changes, loss of smell, exercise intolerance, and loss of taste.

89. The method of any one of claims 86-88, wherein the subject is suffering from an organ failure.

90. The method of claim 89, wherein the organ failure is a renal, hepatic, respiratory, or nervous system failure.

91. The method of claim 90, wherein the subject is being provided ventilatory support.

92. The method of any one of claims 86-91, wherein the subject exhibits an abnormal clinical laboratory measurement selected from the group consisting of: a lymphocyte count outside the range of 4-12×10⁹/L; a hemoglobin level less than 12.3 g/dL; a platelet level outside the range of 150-440×10⁹/L; a C3 level outside the range of 80-178 mg/dL; a C4 level outside the range of 12-42 mg/dL; a CRP level greater than 1 mg/dL; a d-dimer level greater than 500 ng/mL; an IgM level greater than 240 mg/dL; an IgG level greater than 1600 mg/dL; an IgA level greater than 450 mg/dL; a B cell count outside the range of 100-600×10⁶/L; a T cell count outside the range of 0.64-1.18×10⁹/L; a CD4/CD8 T cell ratio greater than 1.0; a natural killer (NK) cell count greater than 100×10⁶/L; an anti-nuclear antibody (ANA) titer greater than 1:160; and any positive autoantibody quantitation to autoantigens listed in Table 4.

93. The method of any one of claims 86-92, wherein the subject exhibits an abnormal clinical laboratory measurement selected from the group consisting of: a blood urea nitrogen (BUN) level greater than 25 mEq/L; a creatine level greater than 1.5 mEq/L; an aspartate transaminase (AST) level greater than 33 U/L; an alanine aminotransferase (ALT) level greater than 36 U/L; an alkaline phosphatase level greater than 115 IU/L; a white blood cell (WBC) count greater than 12×10^{9/}L; a lymphocyte count of about 800 cells per μL; a lactic acid level greater than 4.0 mmol/L; a procalcitonin level greater than 0.25 ng/mL; a lactate dehydrogenase (LDH) level greater than 240 U/L; a ferritin level greater than 284 ng/mL; a c-reactive protein (CRP) level greater than 1.0 mg/dL; and/or a d-dimer level greater than 500 ng/mL.

94. The method of any one of claims 86-93, wherein the subject exhibits an abnormal measurement during an arterial blood gas (ABG) test, wherein optionally the abnormal result comprises an arterial blood pH level outside the range of 7.37-7.44, such as about 7.1; a partial pressure of carbon dioxide (pCO₂) outside the range of 34-43 mm Hg, such as about 60 mm Hg; a partial pressure of oxygen (pO₂) and fraction of inspired oxygen (FiO₂) ratio (pO₂/FiO₂) of less than 300, such as about 255; and a positive end-expiratory pressure (PEEP) of less than 30 cm H₂O.

95. The method of claim 92 or 93, wherein administration of the composition returns the abnormal clinical laboratory measurement in the subject back to a baseline clinical laboratory measurement.

96. The method of claim 95, wherein the baseline clinical laboratory measurement is selected from the group consisting: a BUN level of about 8-25 mEq/L; a creatine level of about 0.5-1.5 mEq/L; an AST level of about 8-33 U/L; an ALT level of about 4-36 U/L; an alkaline phosphatase level of about 25-115 U/L; a WBC count of about 4-12×10⁹/L with about 40-60% neutrophilic predominance and lymphocyte component of about 20-40%; a lactic acid level of about 2-4 mM; a procalcitonin level of about 0.1-0.25 ng/mL; an LDH level of about 50-240 U/L; a ferritin level of about 30-284 ng/mL; a CRP level of about 0.3-1.0 mg/dL; and/or a d-dimer level of less than 500 ng/mL.

97. The method of claim 95, wherein the baseline clinical laboratory measurement is selected from the group consisting: a lymphocyte count of about 4-12×10⁹/L; a hemoglobin level of about 12.3-15.7 g/dL; a platelet level of about 150-440×10⁹/L; a C3 level of about 80-178 mg/dL; a C4 level of about 12-42 mg/dL; a CRP level of about 0.3-1 mg/dL; a d-dimer level of less than 500 ng/mL; an IgM level of about 40-240 mg/dL; an IgG level of about 600-1600 mg/dL; an IgA level of about 80-450 mg/dL; a B cell count of about 100-600×10⁶/L; a T cell count of about 0.64-1.18×10⁹/L; a CD4/CD8 T cell ratio of less than 1.0; a NK cell count greater than 100×10⁶/L; an ANA titer less than 1:160; and no detectable level of autoantigens listed in Table 4.

98. The method of claim 94, wherein, following administration of the composition, the subject exhibits a baseline ABG measurement.

99. The method of claim 98, wherein the baseline ABG measurement comprises a pH level within the range of 7.35-7.45, such as about 7.435; a pCO₂ within the range of 37-45 mm Hg, such as about 43.3 mm Hg; a pO₂/FiO₂ greater than 300, such as about 316; and a PEEP of less than 30 cm H₂O.

100. The method of any one of claims 1-99, wherein administering to the subject the effective amount of the composition resolve one or more symptoms in the subject selected from the group consisting of: cognitive impairment, joint pain, muscle pain, chest pain, stomach pain, headache, tachycardia, numbness, diarrhea, brain fog, fatigue, sleep impairment, dizziness when standing, shortness of breath, fever, skin rash, mood changes, loss of smell, exercise intolerance, and loss of taste.
